SEQUENCE LISTING (6 sequences)

SEQ ID NO: 1 - protein, 449 aa, synthetic construct
QVQLVQSGAEVKKPGASVKVSCKASGYIFISSAMHWVRQAPGQRLEWMGLINPGNGNTKYSQKFQGRVTITRDTSASTAYMELSSLRSEDTAVYYCARAYFRGLGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQSPSSLSASAGDRVTITCRASQSISSYLNWYQQKPGKAPKLLIYAASSLQSGVPSRFSGSGSGTDFTLAISSLQPEDFATYYCQQSYSTPWTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 214 aa, synthetic construct
DIRMTQSPSSLSASAGDRVTITCRASQSISSYLNWYQQKPGKAPKLLIYAASSLQSGVPSRFSGSGSGTDFTLTISSLQPEDFATYYCQQSYSTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 449 aa, synthetic construct; identical to SEQ ID NO: 1

SEQ ID NO: 5 - protein, 216 aa, synthetic construct
QSALTQPASVSGSPGQSITISCTGTSSDVGSYNLVSWYQQHPGKAPKLMIYEGSKRPSGVSNRFSASKSGNTASLTISGLQTEDEADYYCSSYVPRSSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 452 aa, synthetic construct
QVQLVQSGAEVKKPGSSVKVSCKASGGTFSTYTINWVRQAPGQGLEWMGRIIPVLGIANYAQKFQGRVTITADKSTSTAYMELSSLRSEDTAVYYCARDTPRLRSSYYMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVCVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0307549 A1
(43) Pub. Date: Sep. 19, 2024

(54) SUBCUTANEOUSLY ADMINISTERED ANTIBODY-DRUG CONJUGATES FOR USE IN CANCER TREATMENT

(71) Applicant: Heidelberg Pharma Research GmbH, Ladenburg (DE)

(72) Inventors: Michael KULKE, Ladenburg (DE); Torsten HECHLER, Ladenburg (DE); Kristin DECKER, Ladenburg (DE); Andreas PAHL, Ladenburg (DE); András STRASSZ, Ladenburg (DE)

(73) Assignee: Heidelberg Pharma Research GmbH, Ladenburg (DE)

(21) Appl. No.: 18/603,662

(22) Filed: Mar. 13, 2024

(30) Foreign Application Priority Data
Mar. 13, 2023 (EP) 23161638.4

(51) Int. Cl. A61K 47/68; A61K 9/00; A61K 38/47; A61K 45/06; A61P 35/00

(52) U.S. Cl. CPC A61K 47/6831; A61K 9/0019; A61K 38/47; A61K 45/06; A61K 47/6849; A61K 47/6851; A61K 47/6889; A61P 35/00; C12Y 302/01035

(57) ABSTRACT

The present application pertains to pharmaceutical compositions for use in the treatment of cancer, whereby said pharmaceutical compositions are administered subcutaneously and wherein the pharmaceutical compositions of the invention comprise at least one conjugate that comprises an antibody which specifically binds to a cell surface antigen on a cancer cell and at least one amatoxin-linker payload. The present invention further pertains to methods of treating a patient afflicted with cancer using the pharmaceutical compositions of the invention.

### CROSS-REFERENCE TO RELATED APPLICATIONS

This application claims priority to European Application No. EP 23161638.4 filed on Mar. 13, 2023. The content of European Application No. EP 23161638.4 is hereby incorporated by reference in its entirety.

### REFERENCE TO A SEQUENCE LISTING

The present application is accompanied by an XML file as a computer readable form containing the sequence listing entitled, “006033US-seqlist-as-filed.xml”, created on Mar. 12, 2024, with a file size of 8,203 bytes, the content of which is hereby incorporated by reference in its entirety.

### FIELD OF THE INVENTION

The present invention relates to subcutaneous administration of pharmaceutical compositions of antibody-drug conjugates (ADCs) comprising one or more cytotoxic amatoxin moieties conjugated to an antibody or antigen-binding antibody fragment and their respective use in cancer treatment. In a further aspect, the present invention relates to methods of treating cancer in a patient using the pharmaceutical compositions of the invention.

### BACKGROUND

Conventional cancer therapy often entails a low therapeutic window and non-specificity of chemotherapeutic agents that consequently affects normal cells with high mitotic rates and results in a number of adverse effects. Monoclonal antibodies have demonstrated great therapeutical potential for the treatment of several diseases, particularly for cancer. Prior to the development of antibody-drug conjugates (ADCs) for cancer treatment, monoclonal antibodies have received interest, which was attributable to their target specificity, therapeutic index, and generally with fewer side effects when compared to conventional therapy, such as chemotherapy, or chemo-radiation therapy.

The design and development of ADCs entail many challenges, including susceptibility to degradation, or a concentration-dependent propensity for aggregation caused by the hydrophobicity of the respective payload of the ADCs. Attempts to mitigate these limitations by lowering the concentration of ADCs in their respective formulation resulted in limited bioavailability and tissue penetration.

Commonly used ADC toxins, such as auristatins, maytansinoids, and calicheamicins typically have a 100-1000 times higher in vitro potency than that of the traditional chemotherapeutic agents with IC₅₀ values in the subnanomolar range. This increase in potency is necessary to enable effective tumor cell killing by ADCs where the delivery to the intracellular drug target is linked to the antigen expression level on the surface of the tumor cell and internalization efficiency. However, the improvement of in vitro potency is often associated with increased hydrophobicity of the respective payloads.

Hydrophobicity represents an important factor contributing to the overall physicochemical properties of the ADC payload. Although an increase in hydrophobicity often results in an improvement of in vitro potency, it also comes with the risks of poor solubility, metabolic instability, and with a greater probability of nonspecific off-target effects.

Subcutaneous delivery of biotherapeutics has become a valuable alternative to intravenous administration across many disease areas. The choice of the administration route depends on several factors including the convenience for the patient and the pharmacokinetic properties of the drug. Each administration route has its own advantages and disadvantages: i.v. administrations lead to a rapid onset of action and almost 100% bioavailability, but it might also be painful, requires hospitalization and leads to high peak serum levels (e.g. a high Cₘₐₓ) that can trigger toxicity. “Cₘₐₓ,” as used herein, refers to the maximum observed plasma concentration of a given drug, such as e.g. the conjugates of the invention. Subcutaneous (s.c.) administration, which is often used for antibody agents, on the other hand, has the advantage that the administration is very easy and can even be done by the patients themselves.

While the pharmacokinetic profiles of subcutaneous and intravenous formulations differ, subcutaneous administration of antibodies has proven to be effective, safe, well-tolerated, and is generally preferred by patients and healthcare providers, as it reduces drug delivery-related healthcare costs and resource use.

Generally, subcutaneous administration of antibody-based therapeutics, including ADCs is, however, limited by the small volume that can be injected. Consequently, high formulation concentrations of ADCs are needed to administer a therapeutically effective dose. Higher concentrations of ADC formulations comprising hydrophic payloads, however, bear the risk of an increased propensity of aggregation of the ADC which in turn is correlated with toxicity. Aggregation of the ADC is also correlated with a reduced half-life and a narrow therapeutic index. The use of low-concentration formulation of ADCs reduces bioavailability and ability to penetrate the tissue to exert pharmacological effects.

Potential disadvantages of the subcutaneous administration route can be a difficult control of the absorption rate and local irritations or skin toxicities. Severe skin toxicities have been reported for intravenous application (i.v.) of trastuzumab-emtansine which employs a maytansine derivate (DM1) payload. The skin toxicities are likely to be caused by extravasation which is an accidental leakage of intravenously administered drug into the tissue around the vein.

An additional concern for s.c. administration of antibodies and ADCs is the isoelectric point (pI) of the respective antibody or ADC. A pI between 7 and 9 of the antibody renders an antibody positively charged at the physiological pH. Positively charged antibodies present a reduced bioavailability of about 30%, while their negatively charged counterparts demonstrate enhanced bioavailability of up to 70% after s.c. administration (Yadav, et al. J. Biol. Chem. 2015, 290, 29732-29741.) While these findings pertain to antibodies, there is not much information regarding the impact of charge on the bioavailability of ADCs, however, it is likely that positively charged ADCs will equally present with reduced bioavailability.

Strategies to overcome the limitations of low s.c. injectable volumes which are typically between 1-2 ml and poor bioavailability have been developed. The use of recombinant human hyaluronidase PH20 (rHuPH20) in combination with the antibody allows injection of larger volumes subcutaneously of up to 5 ml or more. rHuPH20 works by locally degrading hyaluronan (HA), a large glycosaminoglycan and component of extracellular, pericellular, and intracellular matrices. Hyaluronan is a key component of the skin that forms a gel-like substance with water, creating resistance to bulk fluid flow and limiting large volume s.c. drug delivery, dispersion, and absorption. Combining antibodies with hyaluronidase facilitates bulk fluid flow and improves the pharmacokinetic profile after s.c. administration. Some antibodies have been approved for subcutaneous administration in combination with rHuPH20, such as e.g. Rituximab, (Rituxan Hycela/mAbThera s.c), Trastuzumab (Herceptin Hylecta), and Daratumumab (Darzalex Faspro).

WO 2018/187074 A1 discloses methods of cancer therapy using subcutaneous administration of antibody-drug conjugates comprising SN38 payload. However, no corresponding ADCs have been approved for subcutaneous administration.

Thus, there is still an unmet medical need for highly effective ADCs which are administered subcutaneously to maximize efficacy and minimize toxicity.

### SUMMARY OF THE INVENTION

The inventors surprisingly and unexpectedly found that subcutaneous administration of a pharmaceutical composition for use in the treatment of cancer according to the invention which comprises a conjugate that comprises (i) a target-binding moiety, (ii) at least one amatoxin, and (iii) at least one linker connecting said target binding moiety with said at least one amatoxin, resulted in reduced peak serum levels (reduced Cₘₐₓ) and reduced toxicity.

Thus, it was one object of the present invention to provide a pharmaceutical composition which is administered subcutaneously and comprises (i) a target-binding moiety, (ii) at least one amatoxin, and (iii) at least one linker connecting said target binding moiety with said at least one amatoxin.

Preferably, the pharmaceutical compositions of the invention are administered subcutaneously and comprise an antibody, preferably a monoclonal antibody, or an antigen-binding fragment thereof as target-binding moiety. Preferably, the target-binding moiety of the invention is an IgG isotype antibody.

In a particular object of the invention, the pharmaceutical composition comprises a conjugate which is conjugated to at least one amatoxin via a non-cleavable or cleavable linker. Preferably, the cleavable linker of the conjugate according to the invention is an enzymatically cleavable linker which is preferably a self-immolative linker.

According to a further object of the invention, the antibody of the conjugate of the invention does not induce antibody-dependent cell-mediated cytotoxicity (ADCC), antibody-dependent cellular phagocytosis (ADCP), or complement-dependent cytotoxicity (CDC) and comprises at least one amino acid substitution at position D265, L234, L235, or G236 (according to EU numbering system), particularly, the antibody of the conjugate of the invention comprises the amino acid substitutions L234A, L235A and D265C (according to EU numbering system).

According to further objects of the invention, the at least one linker of the conjugate of the invention which connects the at least one amatoxin to the antibody moiety of the ADC is covalently bound to said antibody moiety via any of the naturally occurring cysteine residues of said antibody, preferably via any of the naturally occurring cysteine residues which form the interchain disulfide bonds of said antibody and/or via a disulfide linkage, or to the engineered cysteine residue D265C (according to EU numbering system) in the Fc region of said antibody.

According to some aspects, the pharmaceutical composition of the invention comprises a conjugate which comprises amatoxin linker moieties according to any of formulae XII to XXII as disclosed herein, whereby the conjugate comprises between 1 to about 8, preferably between 1.5, 2 to about 3, or 3.5, particularly preferred 2 amatoxin-linker moieties.

It is one further object of the present invention to provide pharmaceutical compositions for use in methods for treating cancer.

These and further objects are met with methods and means according to the independent claims of the present invention. The dependent claims are related to specific embodiments.

The invention and general advantages of its features will be discussed in detail below.

### DESCRIPTION OF THE FIGURES

FIG. 1. Markush structure of various amatoxins. The numbers in bold type (1 to 8) designate the standard numbering of the eight amino acids forming the amatoxin. The standard designations of the atoms in amino acids 1, 3, and 4 are also shown (Greek letters α to γ, Greek letters α to δ, and numbers from 1′ to 7′, respectively).

FIG. 2 Comparison of efficacy of s.c. vs. i.v. administered conjugates. (A) Murine prostate cancer model using C4-2 tumor cells: (B, C) comparison of half-life, Cmax and AUC of anti-PSMA ADCs and anti-CD37 upon s.c. and i.v. administration showing that s.c. administration of pharmaceutical compositions of the invention results in an increased half-life, reduced Cₘₐₓ and increased AUC.

FIG. 3 NHP tolerability study of anti-PSMA-(XII) conjugate i.v. vs. s.c. administration. NHP study using Cynomolgus monkeys to address liver damage markers upon s.c. and i.v. administration of anti-PSMA-(XII). Top panel: i.v. administration of anti-PSMA-(XII) at 7.5 mg/kg as indicated, lower panel: s.c. administration of anti-PSMA-(XII) at 7.5 mg/kg and 10 mg/kg as indicated. AST: aspartate aminotransferase; ALT: Alanine aminotransferase; LDH: Lactatdehydrogenase levels in the serum of Cynomolgus monkeys following i.v. or s.c. treatment with amatoxin-conjugate as indicated. Subcutaneous administration of conjugate anti-PSMA-(XII) was better tolerated than the corresponding administration via the i.v. route as indicated by the elevated liver enzymes ALT and AST upon i.v. administration of the conjugate. Crosses mark animals that were euthanized for ethical reasons or which died.

FIG. 4 Efficacy of anti-CD37-(XII) conjugates comparison of i.v. vs. s.c. administration. The efficacy of the conjugate anti-CD37-(XII) as disclosed in the application was compared when administered via i.v. route, or s.c. (A) Raji-Luc model (B) probability of survival plot of a murine MEC2 tumor model.

FIG. 5 Pharmacokinetic and bio-distribution study of anti-PSMA-(XII) in mice. (A) concentrations of conjugate anti-PSMA-(XII) in murine serum upon s.c. administration of the conjugate as indicated; (B) concentrations of conjugate anti-PSMA-(XII) in murine serum upon i.v. administration as indicated. (C) PK data for s.c. and i.v. administration of conjugate anti-PSMA-(XII). The data illustrate that s.c. administration results in a reduction of Cmax, while the AUC largely remains unchanged.

FIG. 6 Pharmacokinetic and bio-distribution study of anti-CD37-(XII) in mice. (A) concentrations of conjugate anti-CD37-(XII) in murine serum upon s.c. administration of the conjugate as indicated; (B) concentrations of conjugate anti-CD37-(XII) in murine serum upon i.v. administration as indicated. (C) PK data for s.c. and i.v. administration of conjugate anti-CD37-(XII). The data illustrate that s.c. administration results in a reduction of Cmax, while the AUC largely remains unchanged.

FIG. 7 Pharmacokinetic study of αPSMA-(XII) conjugate concentration in serum of cynomolgus monkey. (A) Serum concentration of conjugate anti-PSAM-(XII) upon i.v. and s.c. administration as indicated. Intravenous administration results in a higher and faster Cₘₐₓ compared to s.c. administration of the conjugate; (B) Detailed results of Pharmacokinetic study indicating that s.c. administration of the conjugate results in a lower Cₘₐₓ (C_{max(i.v.)} of about 219 μg/ml vs. C_{max(s.c.)} of about 96 μg/ml at a dose of 7.5 mg/kg).

FIG. 8 Comparison of anti tumor efficacy of i.v.- and s.c.-administered anti-GCC conjugates. (A) Anti-tumor efficacy of anti-GCC-LALA-D265C-(XIV) administered at 2.5 mg/kg s.c. (open squares) and 2.5 mg/kg i.v. (filled squares); (B) Anti-tumor efficacy of anti-GCC-LALA-D265C-(XII) administered at 6 mg/kg s.c. (open triangles) and 6 mg/kg i.v. (filled triangles).

FIG. 9 Pharmacokinetics of amanitin-based anti-PSMA-(XIV) ADC. Anti-PSMA ADC h3/F11-LALA-D265C Var16-(XIV) serum concentration over time, following a single s.c. administration of a 10 mg/kg or 5 mg/kg dose or i.v. administration of a 5 mg/kg dose to male CB17-SCID mice.

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values. Recitation of ranges of values herein is merely intended to serve as a shorthand method of referring individually to each separate value falling within the range. Unless otherwise indicated herein, each individual value is incorporated into the specification as if it were individually recited herein.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure and avoid lengthy repetitions. Chemical terminology used throughout the present application shall be construed according to the “Compendium of Chemical Terminology” published by the International Union of Pure and Applied Chemistry, ISBN: 0-9678550-9-8.

Throughout this application the term “about” is used which shall refer to +/−10% of the numerical value with which it is used.

According to a first aspect of the present invention, the present invention relates to a pharmaceutical composition for use in the treatment of cancer, wherein the pharmaceutical composition comprises a conjugate, wherein the conjugate comprises (i) a target-binding moiety, (ii) at least one amatoxin, and (iii) at least one linker connecting said target binding moiety with said at least one amatoxin, wherein the pharmaceutical composition is administered subcutaneously.

The term “pharmaceutical composition” as used herein means a product comprising pharmaceutical excipients such as buffering agents, preservatives and tonicity modifiers together with the active compound or salt thereof, the pharmaceutical composition is useful for treating or preventing a disease or disorder or to reduce the severity thereof by administering the pharmaceutical composition to a mammal, preferably a human.

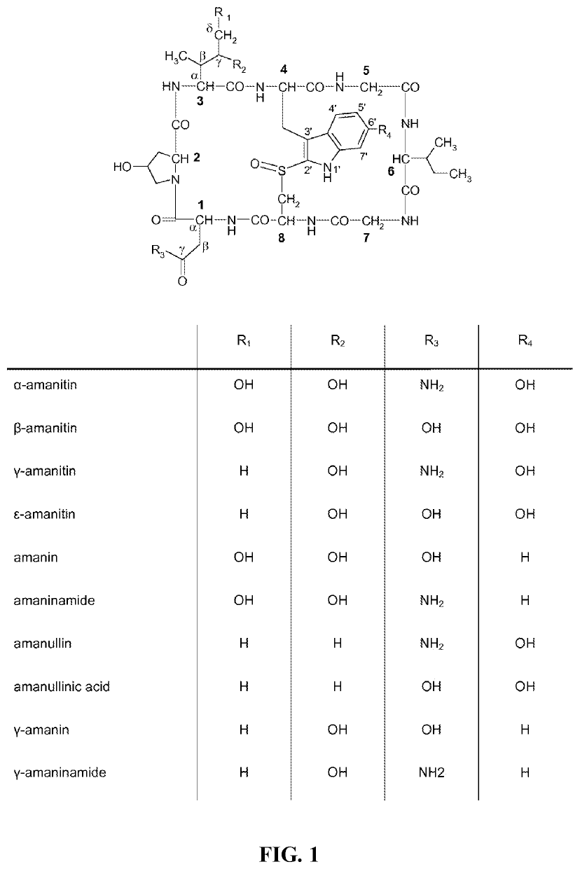

The term “amatoxin” or “amatoxins” as used with the pharmaceutical composition of the invention refers to bicyclic peptides composed of 8 amino acids that are found in Amanita phalloides mushrooms (see FIG. 1). Amatoxins specifically inhibit the DNA-dependent RNA polymerase II of mammalian cells, and thereby also the transcription and protein biosynthesis of the affected cells. Inhibition of transcription in a cell causes stop of growth and proliferation. Though not covalently bound, the complex between amanitin and RNA-polymerase II is very tight (K_{D}=3 nM). Dissociation of amanitin from the enzyme is a very slow process, thus making recovery of an affected cell unlikely. When the inhibition of transcription lasts sufficiently long, the cell will undergo programmed cell death (apoptosis).

In the context of the present invention the term “amatoxin” includes all bicyclic peptides composed of 8 amino acids as isolated from the genus Amanita and described in Wieland, T. and Faulstich H. (Wieland T, Faulstich H., CRC Crit Rev Biochem. 5 (1978) 185-260), further all chemical derivatives thereof; further all semisynthetic analogs thereof; further all synthetic analogs thereof built from building blocks according to the master structure of the natural compounds (cyclic, 8 amino acids), further all synthetic or semisynthetic analogs containing non-hydroxylated amino acids instead of the hydroxylated amino acids, further all synthetic or semisynthetic analogs, in which the sulfoxide moiety is replaced by a sulfone, thioether, or by atoms different from sulfur, e.g., a carbon atom as in a carbanalog of amanitin.

As used herein, a “derivative” of a compound refers to a species having a chemical structure that is similar to the compound, yet containing at least one chemical group not present in the compound it is derived from and/or deficient of at least one chemical group that is present in the compound it is derived from. The compound to which the derivative is compared to is known as the “parent” compound. Typically, a “derivative” may be produced from the parent compound in one or more chemical reaction steps.

As used herein, an “analogue” of a compound is structurally related but not identical to the compound and exhibits at least one activity of the compound. The compound to which the analogue is compared is known as the “parent” compound. The afore-mentioned activities include, without limitation: binding activity to another compound; inhibitory activity, e.g. enzyme inhibitory activity; toxic effects; activating activity, e.g. enzyme-activating activity. It is not required that the analogue exhibits such an activity to the same extent as the parent compound. A compound is regarded as an analogue within the context of the present application, if it exhibits the relevant activity to a degree of at least 1% (more preferably at least 5%, more preferably at least 10%, more preferably at least 20%, more preferably at least 30%, more preferably at least 40%, and more preferably at least 50%) of the activity of the parent compound. Thus, an “analogue of an amatoxin”, as it is used herein, refers to a compound that is structurally related to any one of α-amanitin, β-amanitin, γ-amanitin, ε-amanitin, amanin, amaninamide, amanullin, and amanullinic acid and that exhibits at least 1% (more preferably at least 5%, more preferably at least 10%, more preferably at least 20%, more preferably at least 30%, more preferably at least 40%, 50%, 60%, and more preferably at least 70%, 80%, 90%) of the inhibitory activity against mammalian RNA polymerase II as compared to at least one of α-amanitin, β-amanitin, γ-amanitin, ε-amanitin, amanin, amaninamide, amanullin, and amanullinic acid. An “analogue of an amatoxin” suitable for use in the present invention may even exhibit a greater inhibitory activity against mammalian RNA polymerase II than any one of α-amanitin, β-amanitin, γ-amanitin, ε-amanitin, amanin, amaninamide, amanullin, or amanullinic acid. The inhibitory activity might be measured by determining the concentration at which 50% inhibition occurs (IC₅₀ value). The inhibitory activity against mammalian RNA polymerase II can be determined indirectly by measuring the inhibitory activity on cell proliferation, or alternatively, the inhibitory activity of the amatoxins and their respective derivatives as disclosed herein may e.g. be assessed using RNA polymerase II activity assay as disclosed in Voss et al. BMC Molecular Biology 2014, 15:7.

A “semisynthetic analogue” refers to an analogue that has been obtained by chemical synthesis using compounds from natural sources (e.g. plant materials, bacterial cultures, fungal cultures or cell cultures) as starting material. Typically, a “semisynthetic analogue” of the present invention has been synthesized starting from a compound isolated from a mushroom of the Amanitaceae family. In contrast, a “synthetic analogue” refers to an analogue synthesized by so-called total synthesis from small (typically petrochemical) building blocks. Usually, this total synthesis is carried out without the aid of biological processes.

According to some embodiments of the present invention, the amatoxin can be selected from the group consisting of α-amanitin, β-amanitin, amanin, amaninamide and analogues, derivatives and salts thereof.

Functionally, amatoxins are defined as peptides or depsipeptides that inhibit mammalian RNA polymerase II. Preferred amatoxins are those with a functional group, e.g. a carboxylic group, an amino group, a hydroxy group, a thiol or a thiol-capturing group, that can be reacted with linker molecules or target-binding moieties as defined below.

In the context of the present invention, the term “amanitins” particularly refers to bicyclic structures that are based on an aspartic acid or asparagine residue in position 1, a proline residue, particularly a hydroxyproline residue in position 2, an isoleucine, hydroxyisoleucine or dihydroxyisoleucine in position 3 (or aspartic acid for amanullic acid), a tryptophan or hydroxytryptophan residue in position 4 (or proline for proamanullin), glycine residues in positions 5 and 7 (or isoleucine residues in case of amanullic acid and proamanullin), an isoleucine residue in position 6, and a cysteine residue in position 8, particularly a derivative of cysteine that is oxidized to a sulfoxide or sulfone derivative (for the numbering and representative examples of amanitins, see FIG. 1), and furthermore includes all chemical derivatives thereof; further all semisynthetic analogues thereof; further all synthetic analogues thereof built from building blocks according to the master structure of the natural compounds (cyclic, 8 amino acids), further all synthetic or semisynthetic analogues containing non-hydroxylated amino acids instead of the hydroxylated amino acids, further all synthetic or semisynthetic analogues, in each case wherein any such derivative or analogue is functionally active by inhibiting mammalian RNA polymerase II.

The conjugate of the indention as comprised in the pharmaceutical compositions disclosed herein comprises (i) at least one amatoxin and (ii) at least one linker connecting said target-binding moiety to with said at least one amatoxin. Accordingly, said conjugate comprises one, two, three, four, five, six, seven, eight, nine or ten amatoxin moieties, preferably, said conjugate comprises from about 2 to about 3, or 4 amatoxin moieties which are connected to said target-binding moiety by at least one linker, e.g. each of the amatoxin moieties is connected to said target-binding moiety by a linker such that the number of linkers corresponds to the number of amatoxin moieties. In some embodiments, the pharmaceutical composition of the invention comprises a conjugate as disclosed herein which comprises about two (e.g. from about 1.5 to about 2.5) amatoxin-linker moieties bound to the target-binding moiety, wherein the target-binding moiety is e.g. an antibody, or antigen-binding fragment thereof, preferably the antibody is a monoclonal antibody.

The term “target-binding moiety”, as used herein, refers to any molecule or part of a molecule that can specifically bind to a target molecule or target epitope. Preferred target-binding moieties in the context of the present application are (i) antibodies or antigen-binding fragments thereof; (ii) antibody-like proteins; and (iii) nucleic acid aptamers, (iv) anticalins, or (v) “target-binding moieties” suitable for use in the present invention typically have a molecular mass of 40 000 Da (40 kDa) or more.

A “linker” in the context of the present application refers to a molecule that increases the distance between two components, e.g. to alleviate steric interference between the target binding moiety and the amatoxin, which may otherwise decrease the ability of the amatoxin to interact with RNA polymerase II. The linker may serve another purpose as it may facilitate the release of the amatoxin specifically in the cell being targeted by the target binding moiety. It is preferred that the linker and preferably the bond between the linker and the amatoxin on one side and the bond between the linker and the target binding moiety or antibody on the other side is not cleaved, degraded, or hydrolyzed under the physiological conditions outside the cell, e.g. the blood, while it can be cleaved inside the cell, in particular inside the target cell, e.g. a cancer cell, more specifically inside the lysosome of a cancer cell. To provide this selective stability, the linker may comprise functionalities that are preferably pH-sensitive or protease sensitive. Alternatively, the bond linking the linker to the target binding moiety may provide the selective stability. Preferably a linker has a length of at least 1, preferably of 1-30 atoms length (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 atoms), wherein one side of the linker has been reacted with the amatoxin and, the other side with a target-binding moiety. In the context of the present invention, a linker preferably is a C₁-₃₀-alkyl, C₁-₃₀-heteroalkyl, C₂-₃₀-alkenyl, C₂-₃₀-heteroalkenyl, C₂-₃₀-alkynyl, C₂-₃₀-heteroalkynyl, cycloalkyl, heterocycloalkyl, aryl, heteroaryl, aralkyl, or a heteroaralkyl group, optionally substituted. The linker may contain one or more structural elements such as amide, ester, ether, thioether, disulfide, hydrocarbon moieties and the like. The linker may also contain combinations of two or more of these structural elements. Each one of these structural elements may be present in the linker more than once, e.g. twice, three times, four times, five times, or six times.

In some embodiments the linker may comprise a disulfide bond. It is understood that the linker has to be attached either in a single step or in two or more subsequent steps to the amatoxin and the target binding moiety. To that end the linker to be will carry two groups, preferably at a proximal and distal end, which can (i) form a covalent bond to a group, preferably an activated group on an amatoxin or the target binding-peptide or (ii) which is or can be activated to form a covalent bond with a group on an amatoxin. Accordingly, it is preferred that chemical groups are at the distal and proximal end of the linker, which are the result of such a coupling reaction, e.g. an ester, an ether, a urethane, a peptide bond etc.

As used herein the term “subcutaneous administration” which may also be referred to as “s.c. administration” or “subQ administration” and any grammatical variation thereof refers to the injection of the pharmaceutical composition of the invention into the subcutis, the layer of skin directly below the dermis and epidermis, collectively referred to as the cutis. Subcutaneous administration may e.g. be done by means of a hypodermic needle and a syringe or alternative means such as an autoinjector, or injection pen, such as those e.g. described in WO12085029 A1, or WO20154170 A1, incorporated by reference herein

According to some embodiments, the target-binding moiety of the invention is one of (i) an antibody, preferably a monoclonal antibody, (ii) an antigen-binding fragment thereof, preferably a variable domain (Fv), a Fab fragment or an F(ab)2 fragment, (iii) an antigen-binding derivative thereof, preferably a single-chain Fv (scFv), and (iv) an antibody-like protein. According to preferred embodiments, the target-binding moiety is an antibody, preferably a monoclonal antibody.

As used herein, the term “antibody” shall refer to a protein consisting of one or more polypeptide chains encoded by immunoglobulin genes or fragments of immunoglobulin genes or cDNAs derived from the same. Said immunoglobulin genes include the light chain kappa, lambda and heavy chain alpha, delta, epsilon, gamma and mu constant region genes as well as any of the many different variable region genes.

The basic immunoglobulin (antibody) structural unit is usually a tetramer composed of two identical pairs of polypeptide chains, the light chains (L, having a molecular weight of about 25 kDa) and the heavy chains (H, having a molecular weight of about 50-70 kDa). Each heavy chain is comprised of a heavy chain variable region (abbreviated as VH or VH) and a heavy chain constant region (abbreviated as CH or CH). The heavy chain constant region is comprised of three domains, namely CH1, CH2 and CH3. Each light chain contains a light chain variable region (abbreviated as VL or VL) and a light chain constant region (abbreviated as CL or CL). The VH and VL regions can be further subdivided into regions of hypervariability, which are also called complementarity determining regions (CDR) interspersed with regions that are more conserved called framework regions (FR). Each VH and VL region is composed of three CDRs and four FRs arranged from the amino terminus to the carboxy terminus in the order of FR1, CDR1, FR2, CDR2, FR3, CDR3, FR4. The variable regions of the heavy and light chains form a binding domain that interacts with an antigen.

The CDRs are most important for binding of the antibody or the antigen binding portion thereof. The FRs can be replaced by other sequences, provided the three-dimensional structure which is required for binding of the antigen is retained. Structural changes of the construct most often lead to a loss of sufficient binding to the antigen.

The term “antigen binding portion” of the (monoclonal) antibody refers to one or more fragments of an antibody which retain the ability to specifically bind to a given antigen in its native form. Examples of antigen binding portions of the antibody include a Fab fragment, a monovalent fragment consisting of the VL, VH, CL and CH1 domains, an F(ab′)2 fragment, a bivalent fragment comprising two Fab fragments linked by a disulfid bridge at the hinge region, an Fd fragment consisting of the VH and CH1 domain, an Fv fragment consisting of the VL and VH domains of a single arm of an antibody, and a dAb fragment which consists of a VH domain and an isolated complementarity determining region (CDR).

The antibody, or antibody fragment or antibody derivative thereof, according to the present invention can be a monoclonal antibody. As used herein, the term “monoclonal antibody” (“mAb”) refers to a preparation of antibody molecules of single binding specificity and affinity for a particular epitope, representing a homogenous antibody population, i.e., a homogeneous population consisting of a whole immunoglobulin, or a fragment or derivative thereof. Preferably, such antibody is selected from the group consisting of IgG, IgD, IgE, IgA and/or IgM, or a fragment or derivative thereof, preferably the monoclonal antibody of the invention is of the IgG isotype, e.g. IgG1, or IgG4, more preferably of the IgG1 isotype. The term “antibody” as used with the present invention shall also include and refer to antibody portions, or antibody moieties that are comprised in conjugates according to the present invention.

As used herein, the term “fragment” or “antigen-binding fragment” shall refer to fragments of such antibody retaining target binding capacities, e.g., a CDR (complementarity determining region), a hypervariable region, a variable domain (Fv), an IgG heavy chain (consisting of VH, CH1, hinge, CH2 and CH3 regions), an IgG light chain (consisting of VL and CL regions), and/or a Fab and/or F(ab)2.

As used herein, the term “derivative” or “antigen-binding derivative” shall refer to protein constructs being structurally different from, but still having some structural relationship to, the common antibody concept, e.g., scFv, Fab and/or F(ab)2, as well as bi-, tri- or higher specific antibody constructs, all of which have about the same target-binding specificity as the monoclonal antibodies of the invention All these items are explained below.

Other antibody derivatives known to the skilled person are Diabodies (as disclosed in e.g. Proc Natl Acad Sci USA. 1993 Jul. 15;90(14):6444-8), Camelid Antibodies, Domain Antibodies, bivalent homodimers with two chains consisting of scFvs, IgAs (two IgG structures joined by a J chain and a secretory component), shark antibodies (IgNAR), antibodies consisting of new world primate framework plus non-new world primate CDR, dimerised constructs comprising CH3+VL+VH, other scaffold protein formats comprising CDRs, and antibody conjugates (e.g., antibody, or fragments or derivatives thereof, linked to a drug, a toxin, a cytokine, an aptamer, a nucleic acid such as a desoxyribonucleic acid (DNA) or ribonucleic acid (RNA), a therapeutic polypeptide, a radioisotope or a label).

As used herein, the term “antibody-like protein” refers to a protein that has been engineered (e.g. by mutagenesis of Ig loops) to specifically bind to a target molecule. Typically, such an antibody-like protein comprises at least one variable peptide loop attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Antibody-like proteins include without limitation affilin proteins, affibodies, anti-calins, and designed ankyrin repeat proteins (see e.g. Binz et al., 2005, or WO2012135345A1). Antibody-like proteins can be derived from large libraries of mutants, e.g. by panning from large phage display libraries, and can be isolated in analogy to regular antibodies. Also, antibody-like binding proteins can be obtained by combinatorial mutagenesis of surface-exposed residues in globular proteins.

As used herein, the term “Fab” relates to an IgG fragment comprising the antigen binding region, said fragment being composed of one constant and one variable domain from each heavy and light chain of the antibody.

As used herein, the term “F(ab)2” relates to an IgG fragment consisting of two Fab fragments connected to one another by disulfide bonds.

As used herein, the term “scFv” relates to a single-chain variable fragment being a fusion of the variable regions of the heavy and light chains of immunoglobulins, linked together with a short linker, usually comprising serine (S) and/or glycine (G) residues. This chimeric molecule retains the specificity of the original immunoglobulin, despite removal of the constant regions and the introduction of a linker peptide.

Modified antibody formats are for example bi- or trispecific antibody constructs, antibody-based fusion proteins, immunoconjugates and the like.

IgG, scFv, Fab and/or F(ab)2 are antibody formats which are well known to the skilled person. Related enabling techniques are available from respective textbooks.

According to preferred embodiments of the present invention, said antibody, or antigen-binding fragment thereof or antigen-binding derivative thereof, is a murine, a chimeric, a humanized or a human antibody, or antigen-binding fragment or antigen-binding derivative thereof, respectively, more preferably, said antibody or antigen-binding fragment thereof is a humanized or a human antibody.

Monoclonal antibodies (mAb) derived from mouse may cause unwanted immunological side-effects due to the fact that they contain a protein from another species which may elicit antibodies. In order to overcome this problem, antibody humanization and maturation methods have been designed to generate antibody molecules with minimal immunogenicity when applied to humans, while ideally still retaining specificity and affinity of the non-human parental antibody (for review see Almagro and Fransson 2008, Front Biosci. 2008 Jan. 1;13:1619-33.). Using these methods, e.g., the framework regions of a mouse mAb are replaced by corresponding human framework regions (so-called CDR grafting). WO200907861 discloses the generation of humanized forms of mouse antibodies by linking the CDR regions of non-human antibodies to human constant regions by recombinant DNA technology. U.S. Pat. No. 6,548,640 by Medical Research Council describes CDR grafting techniques, and U.S. Pat. No. 5,859,205 by Celltech describes the production of humanised antibodies.

As used herein, the term “chimeric antibody” relates to an antibody consisting of the antibody's original antigen-binding variable domains with the constant domains being derived from a different species. Since antibodies, in particular monoclonal antibodies, originally most often have been derived from mouse, typically chimeric antibodies comprise human constant domains and mouse variable domains, in order to reduce immunogenicity in humans. Examples of chimeric antibodies used in clinical therapy include infliximab, rituximab and abciximab.

As used herein, the term “humanized antibody” relates to an antibody, a fragment or a derivative thereof, in which at least a portion of the constant regions and/or the framework regions, and optionally a portion of CDR regions, of the antibody is derived from or adjusted to human immunoglobulin sequences. Methods of antibody humanization are known in the art and have been described, for example, in Riechmann et al., Nature 332:323-327, 1988; U.S. Patent Nos: 5,530,101; 5,585,089; 5,693,761; 5,693,762; and 6,180,370.

The antibodies, the antibody fragments or antibody derivatives thereof, disclosed herein can comprise humanized sequences, in particular of the preferred VH- and VL-based antigen-binding region which maintain appropriate ligand affinity. The amino acid sequence modifications to obtain said humanized sequences may occur in the CDR regions and/or in the framework regions of the original antibody and/or in antibody constant region sequences.

Said antibody, or antibody fragment or antibody derivative thereof, can be glycosylated. The glycan can be an N-linked oligosaccharide chain at asparagin 297 of the heavy chain.

The antibodies or fragments or derivatives of the present invention may be produced by transfection of a host cell with an expression vector comprising the coding sequence for the antibody according to the invention. The expression vector or recombinant plasmid is produced by placing the coding antibody sequences under control of suitable regulatory genetic elements, including promoter and enhancer sequences like, e.g., a CMV promoter. Heavy and light chain sequences might be expressed from individual expression vectors which are co-transfected, or from dual expression vectors. Said transfection may be a transient transfection or a stabile transfection. The transfected cells are subsequently cultivated to produce the transfected antibody construct. When stabile transfection is performed, then stable clones secreting antibodies with properly associated heavy and light chains are selected by screening with an appropriate assay, such as, e.g., ELISA, subcloned, and propagated for future production.

According a preferred embodiment, the antibody or antibody-moiety or antigen-binding fragments thereof of the conjugate of the pharmaceutical composition according the invention is an IgG isotype antibody, e.g. an IgG1 isotype, an IgG2 isotype, an IgG3 isotype, or an IgG4 isotype antibody.

The use of antibodies which are characterized by reduced or eliminated effector functions in the conjugate of the pharmaceutical composition for use according to the invention may desirable to prevent for example unwanted cytokine secretion, or killing of cells that express the Fcγ receptor such as macrophages. Accordingly, the antibody or antigen-binding fragments of the invention may also include modifications and/or mutations that alter the properties of the antibodies and/or fragments, such as those which decrease ADCC, ADCP, or complement-dependent cytotoxicity CDC as known in the art. Preferably, ADCC, ADCP and CDC are reduced by at least 90% or more, more preferably by at least 95%, more preferably by at least 97.5%, even more preferred by at least 98%, 99% compared to an antibody comprising a wildtype Fc region. According to preferred embodiments, the pharmaceutical composition for use according to the invention comprises an conjugate which comprises an antibody, whereby the antibody does not induce antibody-dependent cell-mediated cytotoxicity (ADCC), antibody-dependent cellular phagocytosis (ADCP), or complement-dependent cytotoxicity (CDC).

The binding of IgG1 to activating and inhibitory Fcγ receptors (FcγRs) or the first component of complement (C1q) depends on residues located in the hinge region and the CH2 domain. Two regions of the CH2 domain are critical for FcγRs and complement C1q binding, and have unique sequences. Substitution of human IgG1 and IgG2 residues at positions 233-236 and IgG4 residues at positions 327, 330 and 331 greatly reduced ADCC and CDC (Armour, et al., Eur. J. Immunol. 29(8) (1999) 2613-2624; Shields, et al., J. Biol. Chem. 276(9) (2001) 6591-6604, WO 2021/234402 A2).

Accordingly, in one embodiment, the pharmaceutical composition for use according to the invention comprises an antibody moiety, or antigen-binding fragment as part of the conjugate as disclosed herein which comprises a variant Fc region, wherein said variant Fc region comprises at least one amino acid modification relative to a wild-type Fc region, such that said molecule has a reduced affinity for IgG1 Fc receptors FcγRI, FcγRII and FcγRIII as well as to complement component C1q compared to a wild-type Fc region.

Affinity to an Fc region, such as the binding of IgG1 to FcγRs, can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay as disclosed in e.g. Wilkinson et al. PLOS One. 2021; 16(12): e0260954 or other mechanism of kinetics-based assay (e.g., BIACORE™ analysis or Octet™ analysis (forteBIO)), and other methods such as indirect binding assays, competitive binding assays fluorescence resonance energy transfer (FRET), gel electrophoresis and chromatography (e.g. gel filtration).

Thus, the pharmaceutical composition for use according to the invention, comprises a conjugate that comprises an antibody as described herein which has been genetically engineered to comprise a variant Fc region which comprise modification of at least one amino acid residue that directly contacts FcγRs based on structural and crystallographic analysis. The term “genetically engineered” or “genetic engineering” as used herein relates to the modification of the amino acid sequence or part thereof of a given or natural polypeptide or protein, such as e.g. the Fc region of an antibody, in the sense of nucleotide and/or amino acid substitution, insertion, deletion or reversion, or any combinations thereof, by gene technological methods, such as, e.g., site-directed mutagenesis as described in Biochem. J. (1986) Vol. 237: 1-7, or J Biol Chem. (2015) Vol. 290(5): 2577-2592. As used herein, the term “amino acid substitution” or “mutation” relates to modifications of the amino acid sequence of the protein, wherein one or more amino acids are replaced with the same number of different amino acids, producing a protein which contains a different amino acid sequence than the original protein. A conservative amino acid substitution is understood to relate to a substitution which due to similar size, charge, polarity and/or conformation does not significantly affect the structure and function of the protein. Groups of conservative amino acids in that sense represent, e.g., the non-polar amino acids Gly, Ala, Val, Ile and Leu; the aromatic amino acids Phe, Trp and Tyr; the positively charged amino acids Lys, Arg and His; and the negatively charged amino acids Asp and Glu.

The Fc region of said antibody may further comprise at least one cysteine amino acid substitution at sites where the engineered cysteines are available for conjugation but do not perturb immunoglobulin folding and assembly. Corresponding cysteine-substituted or cysteine-engineered antibodies have disclosed in WO2016040856A2, or Junutula, et al., 2008b Nature Biotech., 26(8):925-932; Dornan et al (2009) Blood 114(13):2721-2729; U.S. Pat. Nos. 7,521,541; 7,723,485; WO2009/052249 and WO2016/142049. The preferred cysteine substitution in the Fc region of the inventive antibodies as disclosed herein is D265C (according to EU numbering system), as disclosed in WO2016142049A1.

According to one embodiment, the antibody moiety comprised in the pharmaceutical composition for use according to the invention comprises least one amino acid substitution at position D265, L234, L235, or G236 (according to EU numbering system), preferably two, or three amino acid substitution at said positions.

According to a preferred embodiment, the conjugates of the invention for use in the pharmaceutical composition according to the invention comprises an antibody, or antibody moiety which comprises an Fc region, which comprises at least one amino acid substitution selected from L234A, L234S, L234G, L235A, L235G, L235S, L235T, G236R, D265C, whereby the amino acid numbering is according to EU numbering system. The EU numbering system may also be referred to as “EU index as in Kabat” and refers to the numbering of the human IgG1 EU antibody, which refers to the numbering of the EU antibody of Edelman et al., 1969, Proc Natl Acad Sci USA 63:78-85.

According to a preferred embodiment the antibody or antibody moiety of the conjugate of the invention as disclosed herein comprises the amino acid substitution D265C (according to EU numbering system). It is particularly preferred, that the Fc region of said antibody comprises the amino acid substitutions L234A, L235A and D265C (according to EU numbering system). Corresponding antibodies which comprise said mutations are particularly suited for ADCs which comprise highly toxic payloads, since they are devoid of Fc effector functions (“Fc silenced”) thereby avoiding the killing of for example macrophages which express Fcγ receptors on their cell surface.

According to one embodiment, the linker of said conjugate is connected to said antibody or antibody moiety via any of the naturally occurring cysteine residues of said antibody, preferably via any of the naturally occurring cysteine residues which form the interchain disulfide bonds of said antibody and/or via a disulfide linkage. Corresponding methods for conjugation of a linker as disclosed herein to the antibody moiety of the conjugate of the invention are e.g. disclosed in WO2005/084390 A2.

According to one embodiment, the amatoxin of said conjugate of the inventive pharmaceutical composition for use in the treatment of cancer as disclosed herein is conjugated to said antibody via a cleavable or non-cleavable linker.

A “cleavable linker” according to the invention is understood as comprising at least one cleavage site. As used herein, the term “cleavage site” shall refer to a moiety that is susceptible to specific cleavage at a defined position under particular conditions. Said conditions are, e.g., specific enzymes or a reductive environment in specific body or cell compartments. For example, cleavable linkers are designed to exploit the differences in local environments, e.g., extracellular and intracellular environments, including, for example, pH, reduction potential or enzyme concentration, to trigger the release of the amatoxin in the target cell. Generally, cleavable linkers are relatively stable in circulation, but are particularly susceptible to cleavage in the intracellular environment through one or more mechanisms (e.g., including, but not limited to, activity of proteases, peptidases, and glucuronidases). Cleavable linkers used herein are substantially stable in circulating plasma and/or outside the target cell (e.g. a cancer cell) and may be cleaved at some efficacious rate inside the target cell or in close proximity to the target cell, e.g. in the tumor microenvironment.

Suitable cleavable linkers according to the invention may e.g. include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference). Suitable cleavable linkers may include, for example, chemical moieties such as a hydrazine, a disulfide, a thioether or a dipeptide.

For example, linkers hydrolyzable under acidic conditions may include, hydrazones, semicarbazones, thiosemicarbazones, cis-aconitic amides, orthoesters, acetals, ketals, or the like (see, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety. Such linkers are relatively stable under neutral pH conditions, such as those in the blood, but are unstable at below pH 5.5 or 5.0, the approximate pH of the lysosome.

Linkers which may e.g. be used in conjugates of the invention are cleavable under reducing conditions such as a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio) butyrate) and SMPT (N-succinimidyl-oxycarbonyl-alpha-methyl-alpha-(2-pyridyl-dithio) toluene), SPDB and SMPT see, e.g., Thorpe et al., 1987, Cancer Res. 47:5924-5931; U.S. Pat. No. 4,880,935, the disclosure of each of which is incorporated herein by reference in its entirety.

According to some embodiments, said cleavage site can be cleavable by at least one protease selected from the group consisting of cysteine protease, metalloprotease, serine protease, threonine protease, and aspartic protease.

Cysteine proteases, also known as thiol proteases, are proteases that share a common catalytic mechanism that involves a nucleophilic cysteine thiol in a catalytic triad or dyad.

Metalloproteases are proteases whose catalytic mechanism involves a metal. Most metalloproteases require zinc, but some use cobalt. The metal ion is coordinated to the protein via three ligands. The ligands co-ordinating the metal ion can vary with histidine, glutamate, aspartate, lysine, and arginine. The fourth coordination position is taken up by a labile water molecule.

Serine proteases are enzymes that cleave peptide bonds in proteins; serine serves as the nucleophilic amino acid at the enzyme's active site. Serine proteases fall into two broad categories based on their structure: chymotrypsin-like (trypsin-like) or subtilisin-like.

Threonine proteases are a family of proteolytic enzymes harbouring a threonine (Thr) residue within the active site. The prototype members of this class of enzymes are the catalytic subunits of the proteasome, however, the acyltransferases convergently evolved the same active site geometry and mechanism.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin.

In some embodiments, said cleavable site is cleavable by at least one agent selected from the group consisting of Cathepsin A or B, matrix metalloproteinases (MMPs), elastases, β-glucuronidase and β-galactosidase, preferably Cathepsin B.

In some embodiments, said cleavage site is a disulfide bond and specific cleavage is conducted by a reductive environment, e.g., an intracellular reductive environment, such as, e.g., acidic pH conditions. For example, a corresponding linker may have the following structure:

(amatoxin)-(CH₂)₂—S—S—(CH₂)₂—X—S-(antibody)

(amatoxin)-(CH₂)₃—S—S—(CH₂)₂—X—S-(antibody);

(amatoxin)-(CH₂)₂—S—S—(CH₂)₃—X—S-(antibody);

(amatoxin)-(CH₂)₃—S—S—(CH₂)₃—X—S-(antibody),

whereby X is as disclosed above.

In some embodiments, the linker is a pH-sensitive linker, and is sensitive to hydrolysis at certain pH values. Typically, the pH-sensitive linker is cleavable under acidic conditions. This cleavage strategy generally takes advantage of the lower pH in the endosomal (pH˜5-6) and lysosomal (pH˜4.8) intracellular compartments, as compared to the cytosol (pH˜7.4), to trigger hydrolysis of an acid labile group in the linker, such as a hydrazone (Jain et al. (2015) Pharm Res 32:3526-40). In some embodiments, the linker is an acid labile and/or hydrolyzable linker. For example, an acid labile linker that is hydrolyzable in the lysosome, and contains an acid labile group (e.g., a hydrazone, a semicarbazone, a thiosemicarbazone, a cis-aconitic amide, an orthoester, an acetal, a ketal, or the like) can be used. See, e.g., U.S. Pat. Nos. 5,122,368; 5,824,805; 5,622,929; Dubowchik and Walker (1999) Pharm. Therapeutics 83:67-123; Neville et al. (1989) Biol. Chem. 264: 14653-61. Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond). See, e.g., U.S. Pat. No. 5,622,929.

According to some embodiments, said cleavable linker of the invention is a enzymatically cleavable linker. Enzymatically cleavable linkers comprise a cleavage site that is an enzymatically cleavable moiety comprising two or more amino acids. Preferably, said enzymatically cleavable moiety comprises a phenylalanine-lysine (Phe-Lys), valine-lysine (Val-Lys), phenylalanine-alanine (Phe-Ala), valine-alanine (Val-Ala), phenylalanine-citrulline (Phe-Cit), or valine-citrulline (Val-Cit) dipeptide, or e.g. a valine-alanine-valine (Val-Ala-Val), leucine-alanine-leucine (Leu-Ala-Leu), glycine-phenylalanine-lysine (Gly-Phe-Lys), isoleucine-alanine-leucine (Ile-Ala-Leu) tripeptide, or e.g. Asp-cBu-Cit, iGlu-cBu-Ala, iGlu-cBu-Cit, iGlu-Val-Ala, Asp-Val-Cit, iGlu-Val-Cit, Ala-Ala-Asn, Glu-Val-Ala, Glu-Val-Cit, Gly-Gly-Phe-Gly, or e.g. a phenylalanine-lysine-glycine-proline-leucin-glycine (Phe Lys Gly Pro Leu Gly) or alanine-alanine-proline-valine (Ala Ala Pro Val) peptide, or a β-glucuronide or β-galactoside.

In some embodiments, the enzymatically cleavable linker according to the invention is a β-glucuronic acid-based linker. Facile release of the drug may be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based linkers may be used to circumvent the tendency of an conjugate of the invention to undergo aggregation due to the hydrophilic nature of β-glucuronides. Corresponding linkers are e.g. disclosed in WO2007011968A2, or β-galactoside-cleavable linker are disclosed in WO19192979 A1, the content of which is incorporated herein by reference.

In some embodiments, the cleavable linker of the conjugates of the invention as disclosed herein is a self-immolative linker. The term “self-immolative linker” or “self-immolative spacer” refers to a bifunctional chemical moiety that is capable of covalently linking two chemical moieties into a normally stable tripartate molecule. The self-immolative spacer is capable of spontaneously separating from the second moiety if the bond to the first moiety is cleaved. Said linker of the conjugate of the invention as disclosed herein includes a “self-immolative” group such as the aforementioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; W098/13059; US20040052793; U.S. Pat. Nos. 6,677,435; 5,621,002; US20040121940; W02004/032828, or WO2005/112919. Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

In preferred embodiments, the enzymatically cleavable linker according to the invention is a cathepsin B cleavable linker and comprises a dipeptide selected from Phe-Lys, Val-Lys, Phe-Ala, Val-Ala, Phe-Cit and Val-Cit, or a tripeptide selected from valine-alanine-valine (Val-Ala-Val), leucine-alanine-leucine (Leu-Ala-Leu), glycine-phenylalanine-lysine (Gly-Phe-Lys), isoleucine-alanine-leucine (Ile-Ala-Leu) tripeptide, a phenylalanine-lysine-glycine-proline-leucin-glycine (Phe Lys Gly Pro Leu Gly) or alanine-alanine-proline-valine (Ala Ala Pro Val) peptide.

In particularly preferred embodiments, the enzymatically cleavable linker according to the invention is a cathepsin B cleavable linker and comprises a dipeptide selected from Phe-Lys, Val-Lys, Phe-Ala, Val-Ala, Phe-Cit and Val-Cit, particularly wherein the cleavable linker further comprises a p-aminobenzyl (PAB) spacer between the dipeptides and the amatoxin as disclosed hereinbelow, whereby the wavy lines indicate attachment sites to the amatoxin and antibody of the conjugate as disclosed herein:

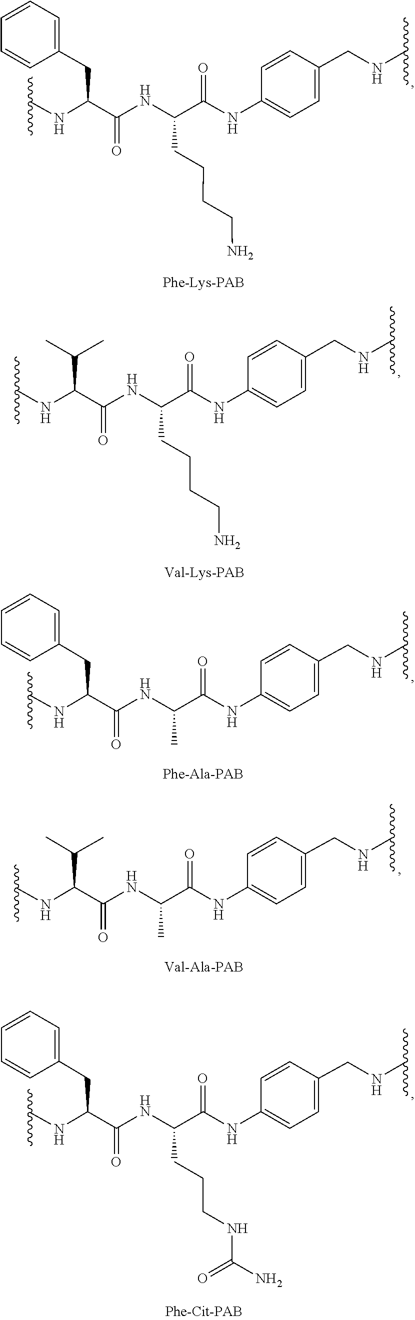 [SMILES: *NCc1ccc(NC(=O)[C@H](CCCCN)NC(=O)[C@H](Cc2ccccc2)N*)cc1 ; *NCc1ccc(NC(=O)[C@H](CCCCN)NC(=O)[C@@H](N*)C(C)C)cc1 ; *NCc1ccc(NC(=O)[C@H](C)NC(=O)[C@H](Cc2ccccc2)N*)cc1 ; *NCc1ccc(NC(=O)[C@H](C)NC(=O)[C@@H](N*)C(C)C)cc1 ; *NCc1ccc(NC(=O)[C@H](CCCNC(N)=O)NC(=O)[C@H](Cc2ccccc2)N*)cc1]

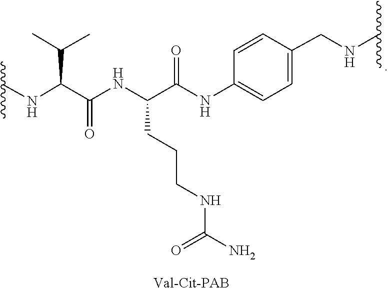 [SMILES: *NCc1ccc(NC(=O)[C@H](CCCNC(N)=O)NC(=O)[C@@H](N*)C(C)C)cc1]

Accordingly, the conjugates as disclosed herein which are comprised in the pharmaceutical composition of the invention. comprise e.g. an enzymatically cleavable moiety which comprises any one of the dipeptides-PAB moieties Phe-Lys-PAB, Val-LysPAB, Phe-Ala-PAB, Val-Ala-PAB, Phe-Cit-PAB, or Val-Cit-PAB as disclosed above.

Preferably, the cleavable moiety of the conjugates of the invention comprises the dipeptide-PAB moiety Val-Ala-PAB

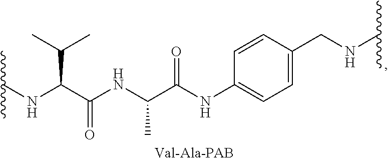 [SMILES: *NCc1ccc(NC(=O)[C@H](C)NC(=O)[C@@H](N*)C(C)C)cc1]

whereby the PAB moiety is linked to the amatoxin.

According to some embodiments, said cleavable linkers of the invention as disclosed herein comprise a thiol-reactive group, selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5-nitropyridine-2-thiol, methanethiosulfonate, or a maleimide.

According to a preferred embodiment the thiol reactive group is a maleimide (meleimidyl moiety) as depicted below:

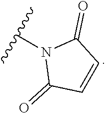 [SMILES: *N1C(=O)C=CC1=O]

Linkers (e.g. cleavable and/or non-cleavable linkers) which comprise said thiol-reactive groups are particularly useful for covalent coupling of linker-amatoxin conjugates as disclosed herein to antibodies comprising reactive thiols, such as e.g. cysteine-engineered antibodies comprising at least one reactive cysteine residue for coupling.

According to a particularly preferred embodiment, the linker of the invention comprises the structure (i) prior to coupling, or (ii) following the coupling to a target-binding moiety, such as an antibody as disclosed herein:

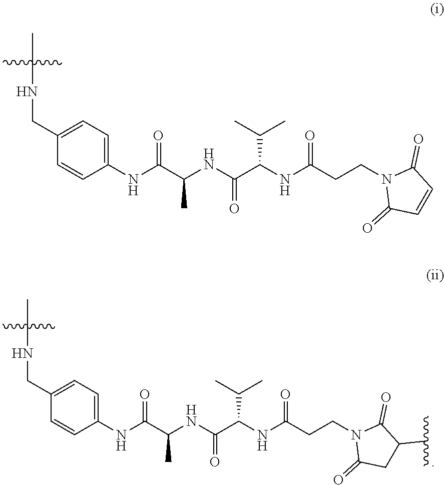 [SMILES: *NCc1ccc(NC(=O)[C@H](C)NC(=O)[C@@H](NC(=O)CCN2C(=O)C=CC2=O)C(C)C)cc1 ; *NCc1ccc(NC(=O)[C@H](C)NC(=O)[C@@H](NC(=O)CCN2C(=O)CC(*)C2=O)C(C)C)cc1]

According to some embodiments, the linker of the conjugate of the invention is a non-cleavable linker. A “non-cleavable linker” is understood not to be subject to enzymatical cleavage by e.g. cathepsin B and is released from the conjugates of the invention during degradation (e.g., lysosomal degradation) from the antibody moiety of the conjugate of the invention inside the target cell. Non-cleavable linkers suitable for use according to the invention may e.g. include one or more groups selected from a bond, —(C═O)—, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include (CH₂)ₚ, (C═O)(CH₂)ₚ, and polyethyleneglycol (PEG; (CH₂CH₂O)ₚ), units, wherein p is an integer from 1-6, independently selected for each occasion.

In some embodiments, the non-cleavable linker according to the invention comprises one or more of a bond, —(C═O)—, a —C(O)NH— group, an —OC(O)NH— group, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, a —(CH₂CH₂O)p- group where p is an integer from 1-6, wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

For example, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene of the non-cleavable linker as disclosed herein may optionally be interrupted by one or more heteroatoms selected from O, S and N and may e.g. be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

According to preferred embodiments, the non-cleavable linker of the conjugate of the invention comprises a —(CH₂)ₙ— unit, where n is an integer from, 2-12, e.g., 2, 3, 4, 5, 6, 7, 8, 9, or 10, or preferably wherein n is an integer from 2-6, e.g. n is 1, 2, 3, 4, 5, or 6.

In a preferred embodiment, the non-cleavable linker of the conjugate of the invention comprises —(CH₂)ₙ— wherein n is 6 and the linker is represented by the formula:

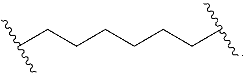 [SMILES: *CCCCCC*]

In some embodiments, the non-cleavable linkers of the invention as disclosed herein further comprise a thiol-reactive group. The thiol-reactive group of said non-cleavable linkers as disclosed above may e.g. be selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5-nitropyridine-2-thiol, methanethiosulfonate, or a maleimide.

According to a preferred embodiment, the thiol reactive group is a maleimide (meleimidyl moiety) as disclosed above. For example, the non-cleavable linker comprising said maleimide may e.g. have the following structure, whereby the wavy line at the linker terminus indicates the point of attachment to the amatoxin:

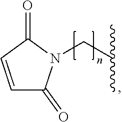

wherein n is an integer from 2-12, e.g. n is 2, 3, 4, 5, 6, 7, 8, 9, or 10, preferably wherein n is an integer from 2-6, e.g. n is 1, 2, 3, 4, 5, or 6, more preferably, n is 6.

Following conjugation to a reactive sulfhydryl on an antibody e.g. a naturally occurring cysteine residues of the antibody, or an engineered cysteine residues, the meleimidyl moiety of e.g. cleavable or non-cleavable linker as disclosed herein comprise the structure:

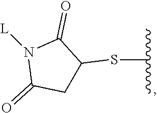

whereby the wavy line represents the attachment site of a cleavable or non-cleavable linker (L) as disclosed herein and the sulfur atom is part of a reactive cysteine of the antibody.

According to preferred embodiments, the conjugate of the invention comprising a cleavable or non-cleavable linker as disclosed herein and which further comprise a thiol-reactive group may be coupled to a naturally occurring sulfhydryl moiety in the antibody of the conjugate, or said cleavable or non-cleavable linker of the conjugates of the invention comprising a thiol-reactive group may be coupled to a sulfhydryl moiety which has been introduced into the antibody by genetic engineering as described in e.g. Nat Biotechnol. 2008 August;26(8):925-32, or WO2006/034488 A2. Preferably, the cleavable or non-cleavable linker as disclosed herein which comprise a thio-reactive group are coupled to sulfhydryl moieties that have been introduced into the Fc region of the respective antibody of the conjugate according the invention by genetic engineering such as e.g. D265C (according to EU numbering)

In some embodiments the at least one amatoxin and at least one linker of the conjugate of the pharmaceutical composition of the invention as disclosed herein is represented by formula (Ia):

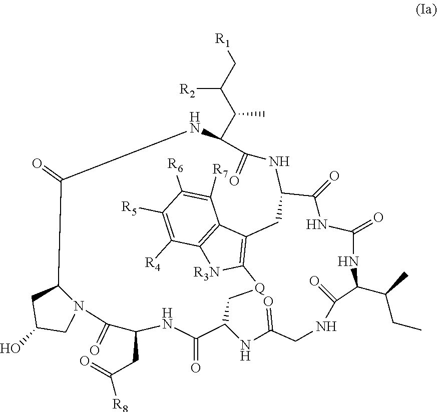

wherein:
R₁ is H, OH, OR_{A}, or OR_{C};
R₂ is H, OH, OR_{B}, or OR_{C};
R_{A} and R_{B}, when present, together with the oxygen atoms to which they are bound, combine to form a 5-membered heterocycloalkyl group;
R₃ is H, R_{C}, or
each of R₄, R₅, R₆, and R₇ is independently H, OH, OR_{C}, R_{C},
R₈ is OH, NH₂, OR_{C}, or NHR_{C},
Q is —S—, —S(O)—, or —SO₂—;
R_{C} is -

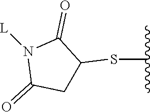

wherein the sulfur atom is part of a reactive cysteine of the antibody and wherein L is a linker (e.g. a cleavable, or non-cleavable linker as defined herein) and is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl; or —((CH₂)ₘO)ₙ(CH₂)ₘ—, where m and n are each independently selected from 1, 2, 3, 4, 5, 6, 7, 8, 9, and 10.

In some embodiments, R_{A} and R_{B}, together with the oxygen atoms to which they are bound, combine to form a 5-membered heterocycloalkyl of formula:

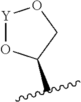

wherein Y is —(C═O)—, —(C═S)—, —(C═NR_{E)}—, or —(CR_{E}R_{E′})—; and
wherein R_{E} and R_{E′} are each independently H, C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C}, or a combination thereof, wherein each C₁-C₆ alkylene-R_{C}, C₁-C₆ heteroalkylene-R_{C}, C₂-C₆ alkenylene-R_{C}, C₂-C₆ heteroalkenylene-R_{C}, C₂-C₆ alkynylene-R_{C}, C₂-C₆ heteroalkynylene-R_{C}, cycloalkylene-R_{C}, heterocycloalkylene-R_{C}, arylene-R_{C}, or heteroarylene-R_{C} is optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, in at least one amatoxin according to formula (Ia) as disclosed above
R₁, R₂ and R₉ are OH.
R₃ is H, R_{C}, or R_{D};
each of R₄, R₅, R₆, and R₇ is independently H, OH, OR_{C}, R_{C},
R₈ is OH, NH₂, OR_{C}, NHR_{C},
Q is —S—, —S(O)—, or —SO₂—;
R_{C} is as disclosed above.

In some embodiments the at least one amatoxin and at least one linker of the conjugate of the pharmaceutical composition of the invention as disclosed herein is represented by formula (IIa):

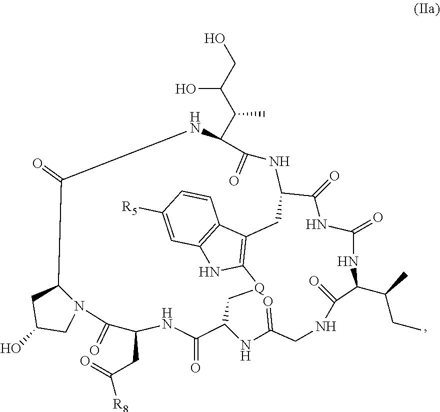

wherein R₁, R₂ and R₉ are OH.
R₅, is independently H, OH, OR_{C}, R_{C},
R₈ is OH, NH₂, OR_{C}, NHR_{C}, or NR_{C}
Q is —S—, —S(O)—, or —SO₂—;
R_{C} is -

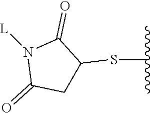

wherein the sulfur atom is part of a reactive cysteine of the antibody and wherein L is a linker e.g. non-cleavable or cleavable linker as defined hereinbelow, and is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl; or comprises a dipeptide; or —((CH₂)ₘO)ₙ(CH₂)ₘ—, where m and n are each independently selected from 1, 2, 3, 4, 5, 6, 7, 8, 9, and 10; and; or L is a cleavable linker, preferably an enzymatically cleavable linker, more preferably a self-immolative cathepsin B cleavable linker as disclosed herein.

According to some embodiments, the linker of the conjugate of the invention as disclosed herein is connected to the at least one amatoxin via (i) the γ C-atom of amatoxin amino acid 1, or (ii) the δ C-atom of amatoxin amino acid 3, or (iii) the 6′-C-atom of amatoxin amino acid 4.

In a one embodiment of the present invention, said pharmaceutical composition of the invention comprises a conjugate as described herein, which comprises an amatoxin comprising (i) an amino acid 4 with a 6′-deoxy position and (ii) an amino acid 8 with an S-deoxy position.

According to particularly preferred embodiments of the present invention, said pharmaceutical composition for use in the treatment of cancer according to the invention comprises a conjugate which comprises at least one of the following compounds of formulae (I) to (XI), respectively, as linker-amatoxin moieties:

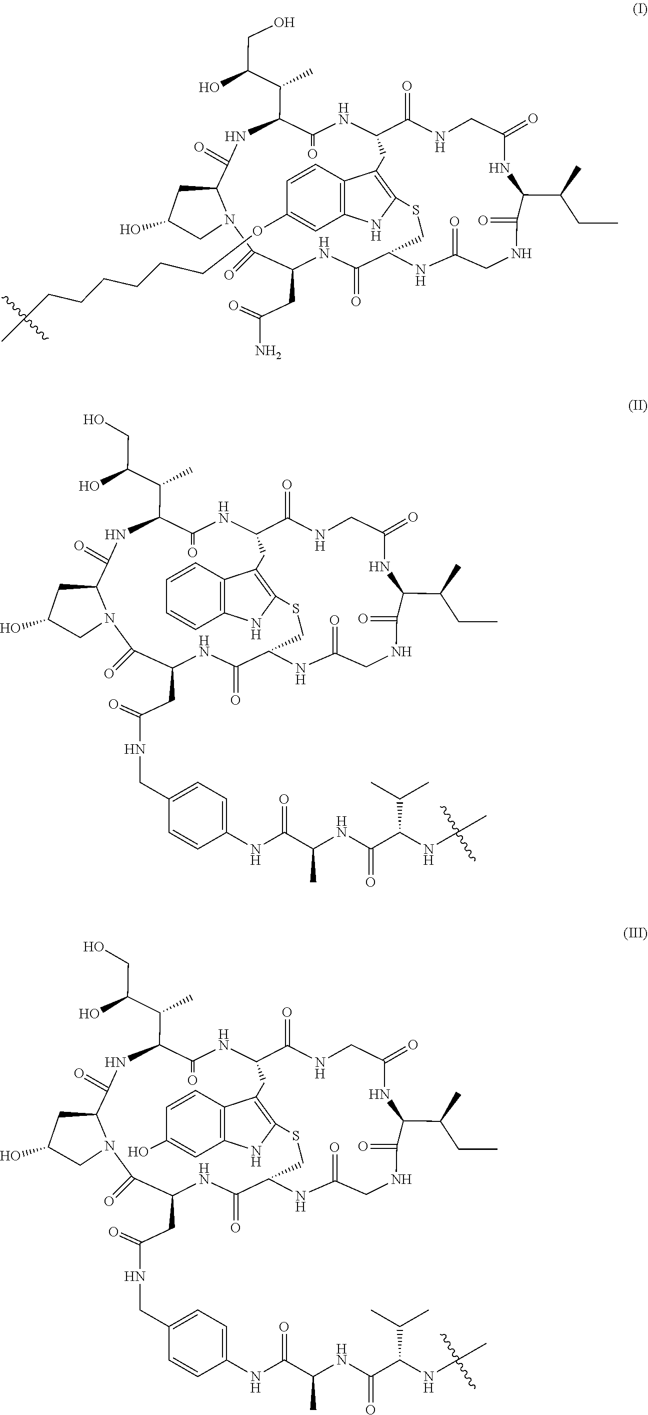

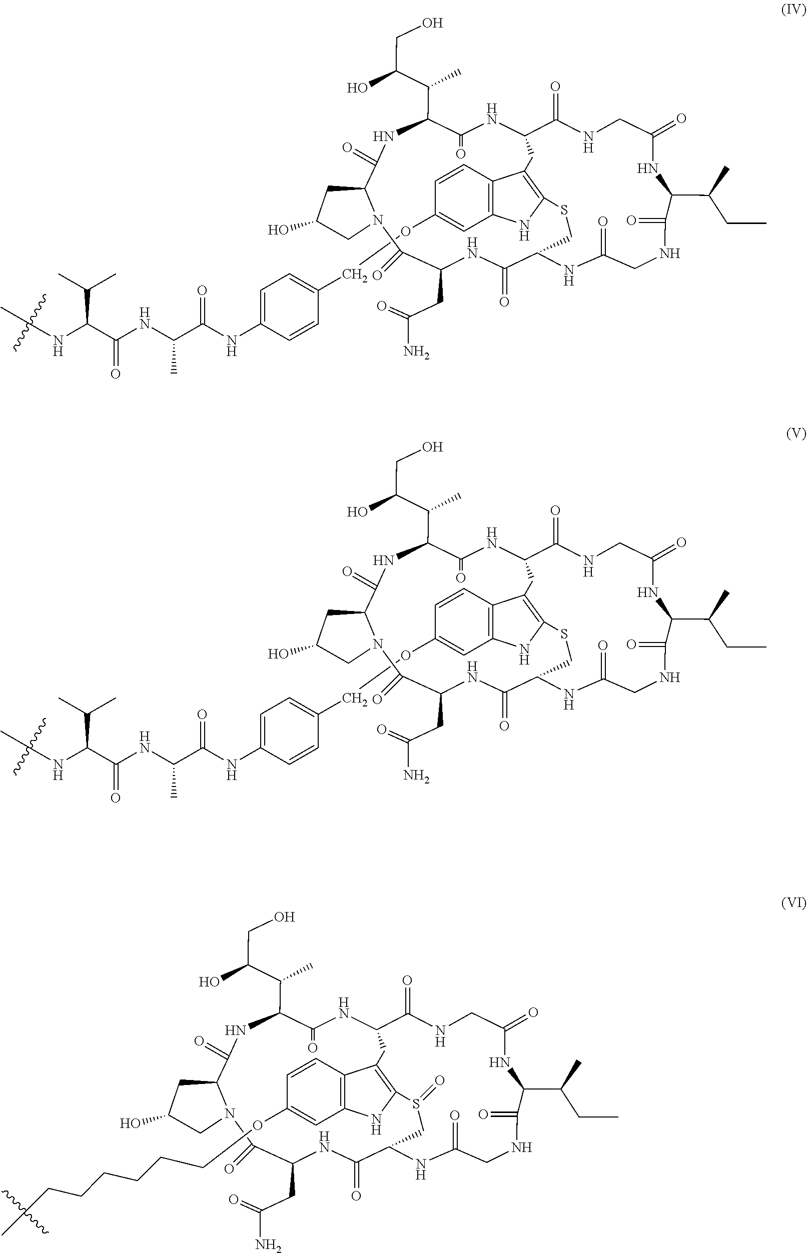

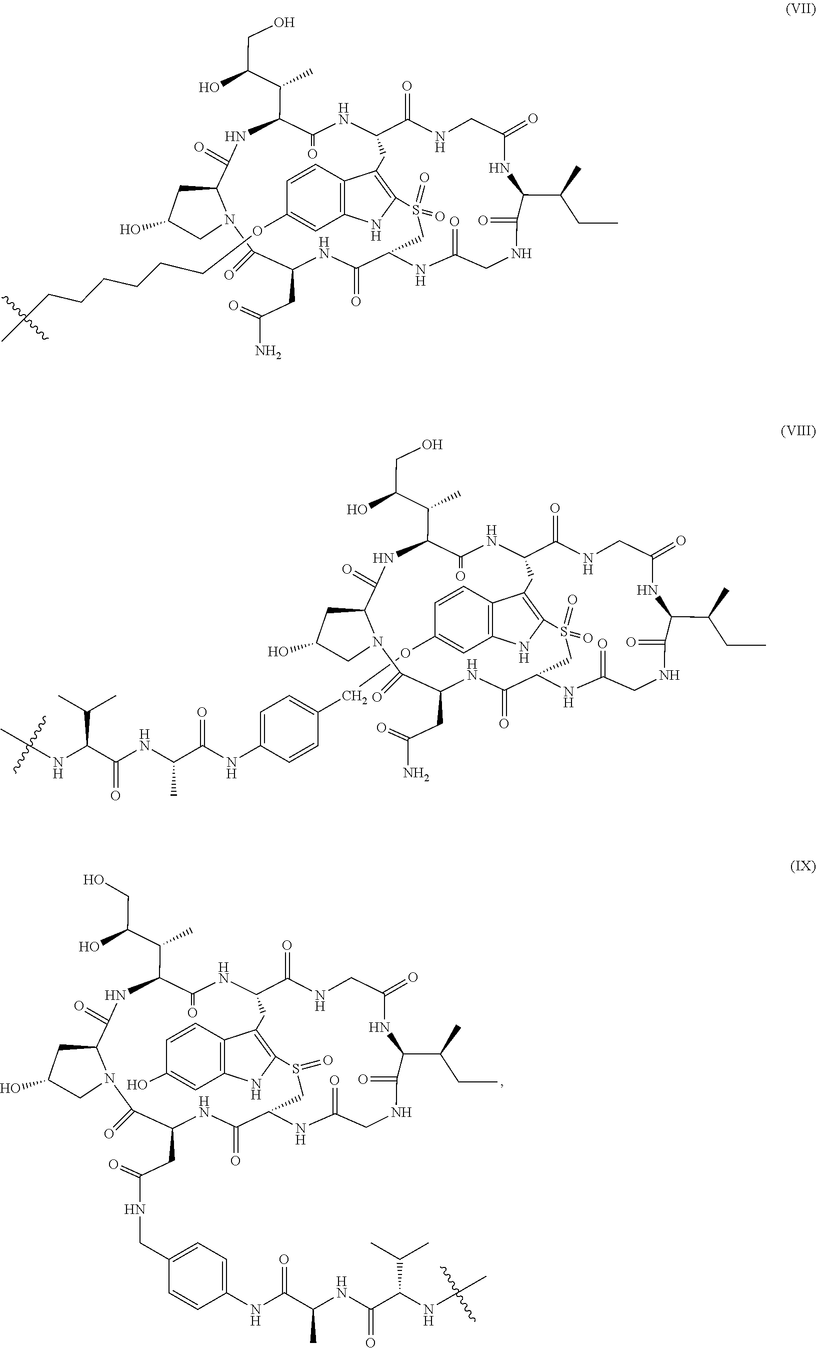

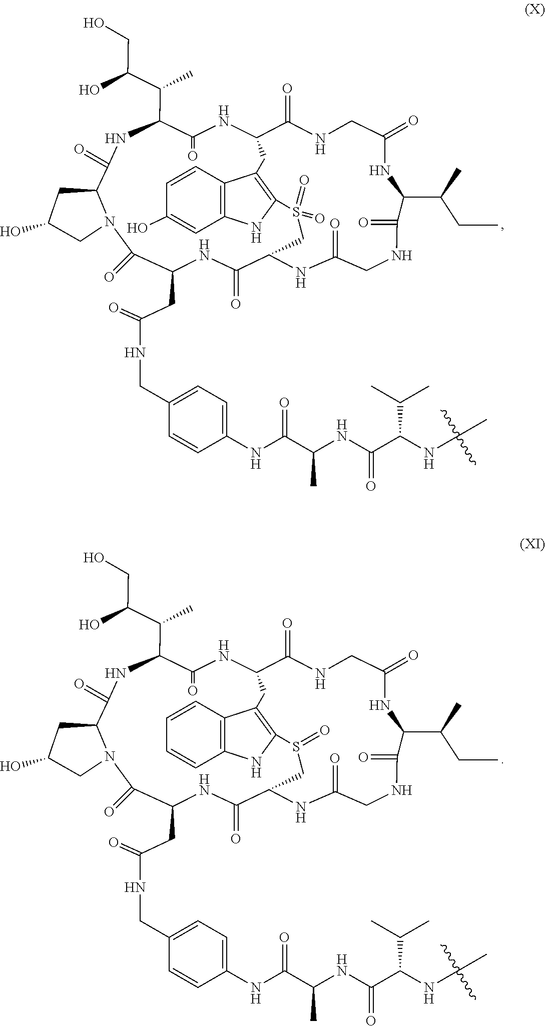 [SMILES: *N[C@H](C(=O)N[C@@H](C)C(=O)Nc1ccc(CNC(=O)C[C@@H]2NC(=O)[C@@H]3CS(=O)(=O)c4[nH]c5cc(O)ccc5c4C[C@H](NC(=O)[C@H]([C@@H](C)[C@@H](O)CO)NC(=O)[C@@H]4C[C@@H](O)CN4C2=O)C(=O)NCC(=O)N[C@@H]([C@@H](C)CC)C(=O)NCC(=O)N3)cc1)C(C)C ; *N[C@H](C(=O)N[C@@H](C)C(=O)Nc1ccc(CNC(=O)C[C@@H]2NC(=O)[C@@H]3CS(=O)c4[nH]c5ccccc5c4C[C@H](NC(=O)[C@H]([C@@H](C)[C@@H](O)CO)NC(=O)[C@@H]4C[C@@H](O)CN4C2=O)C(=O)NCC(=O)N[C@@H]([C@@H](C)CC)C(=O)NCC(=O)N3)cc1)C(C)C]

Accordingly, the pharmaceutical composition for use in the treatment of cancer of the invention comprise a conjugate which comprises an antibody covalently bound to at least one, e.g. one, two, three, four, five, six, seven, or eight, of the amatoxin-linker conjugates (I)-(XI), preferably from about 1 to about 4, or from about 3 to about 6, preferably from about 2 to about 3, more preferably about two amatoxin-linker conjugates (I)-(XI).

According to preferred embodiments, the pharmaceutical composition for use in the treatment of cancer according to the invention comprises a conjugate which comprises an antibody covalently bound conjugated to amatoxin linker moieties via a thioether linkage according to any one of formula XII to XXII:

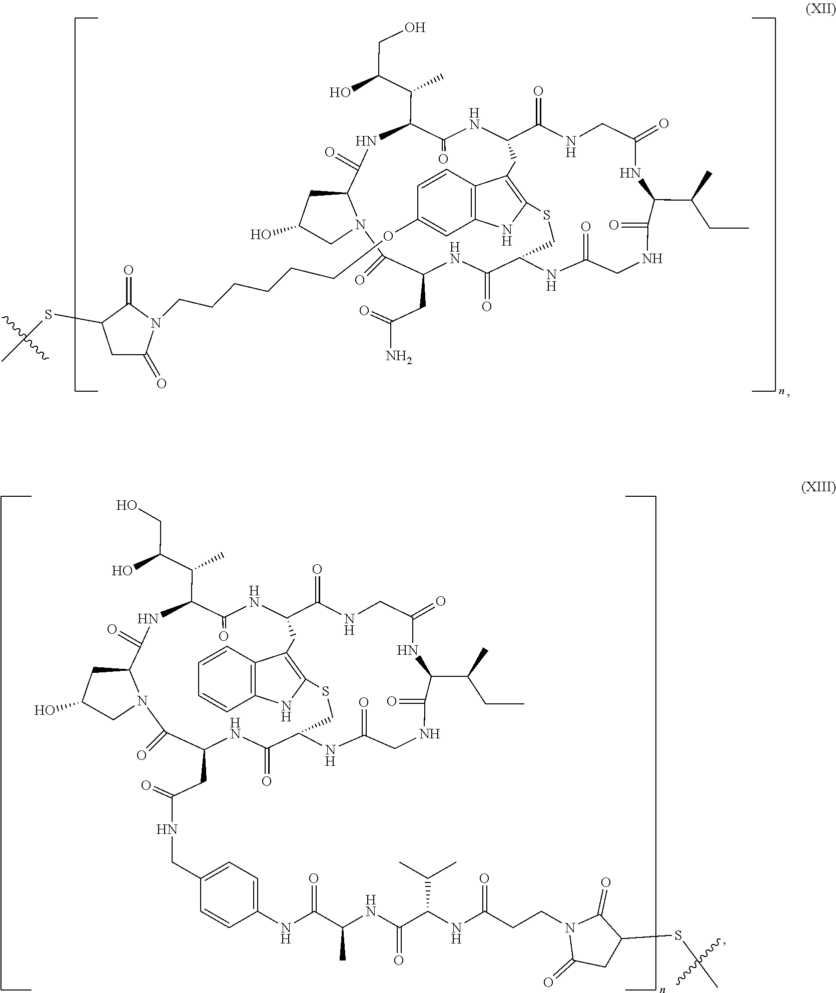

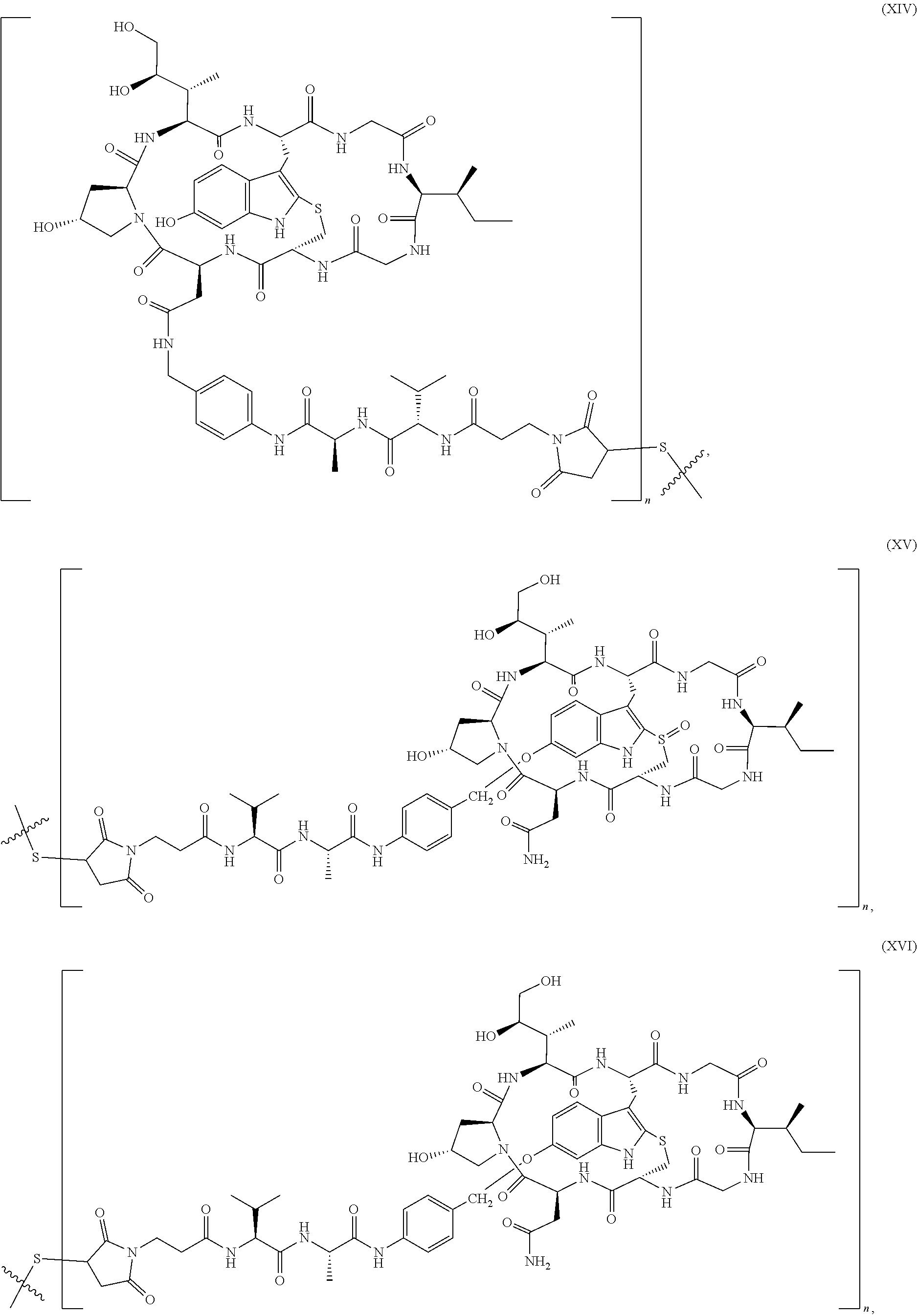

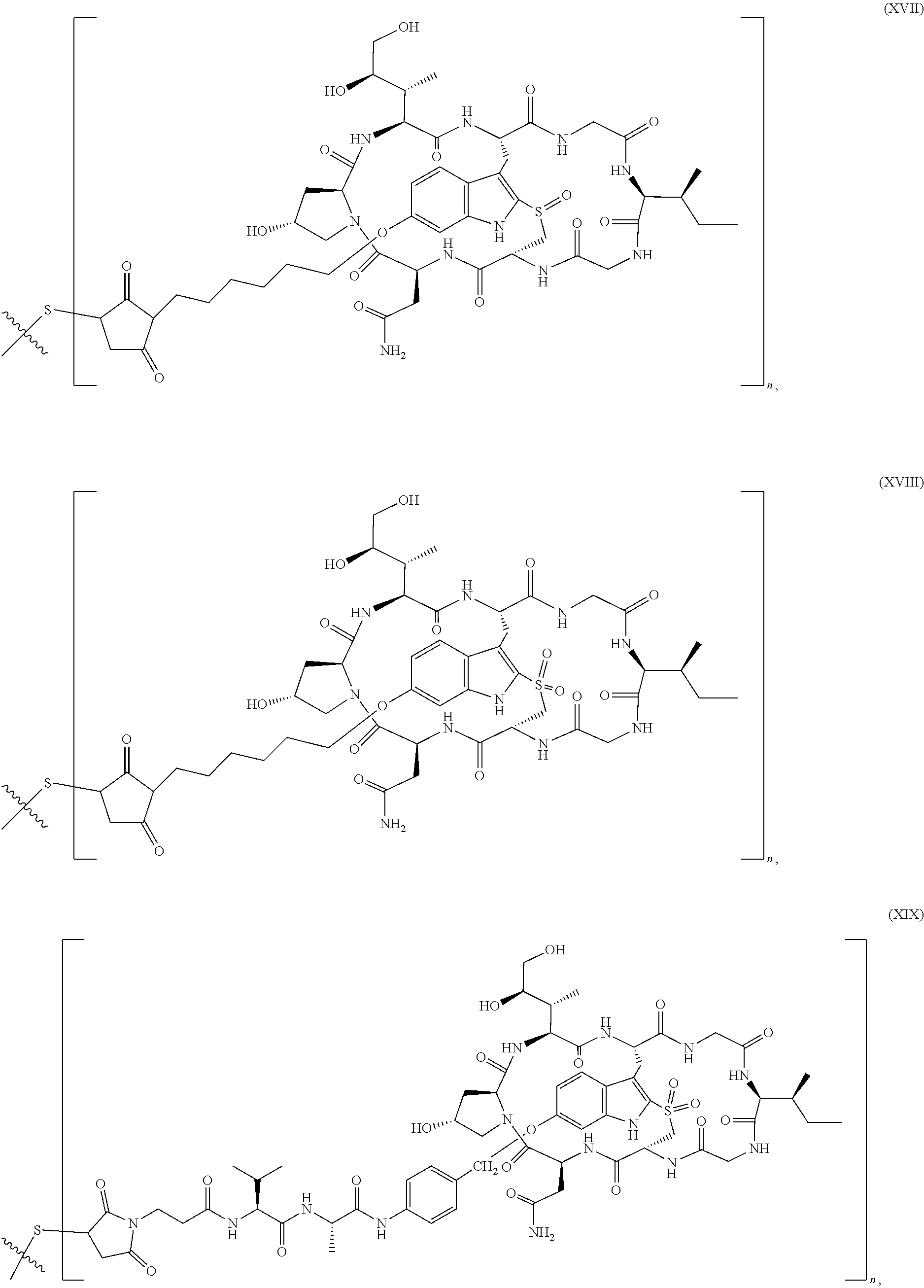

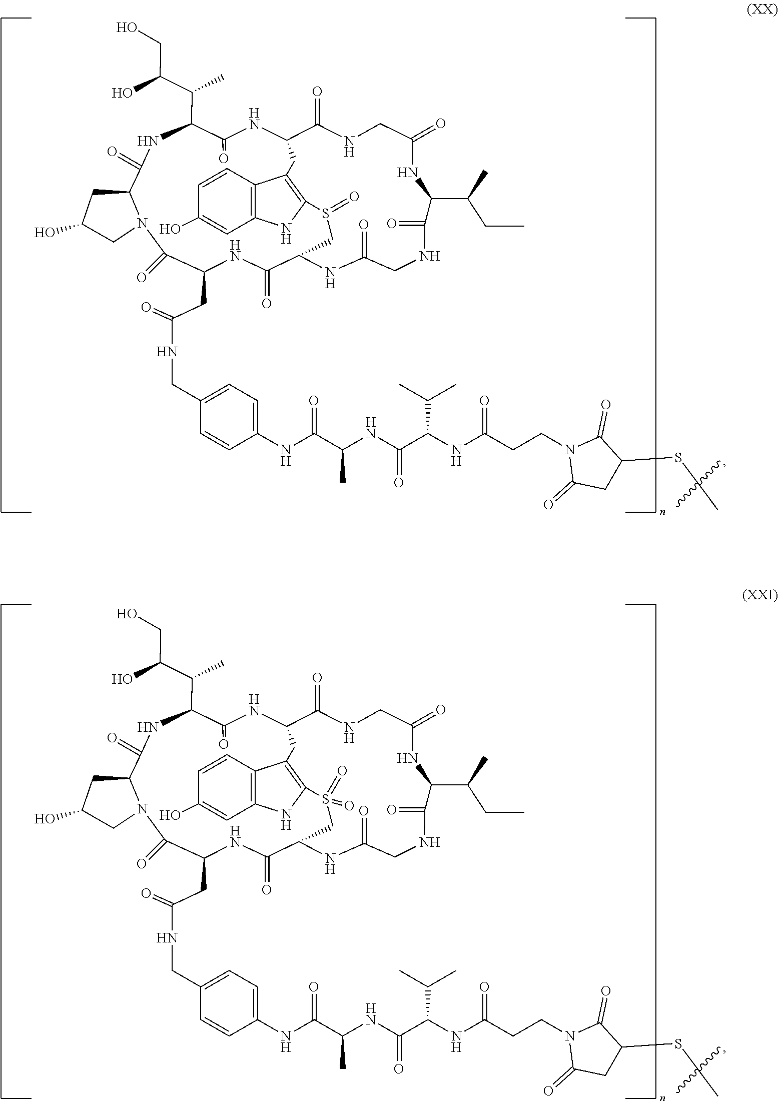

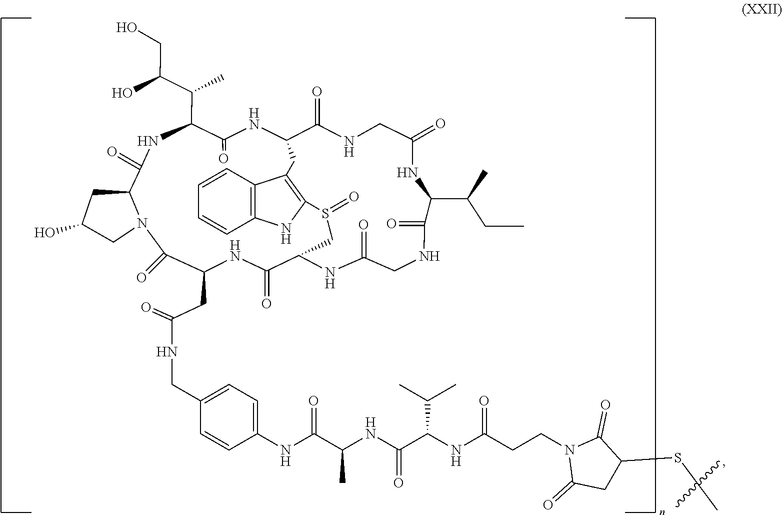

wherein said amatoxin-linker moieties are coupled to the thiol groups of cysteine residues of antibody or antibody moiety of the conjugate according to the invention, and wherein n is preferably from about 1, 2, 3, to about 4, 5, 6, 7, 8, preferably, wherein n is from about 1, 1.5, 2, 2.5 to about 3.5, 4.5, 5.5, more preferably, wherein n is from about 1.5 to about 3.5, most preferably wherein n is about 2. The thiol groups of cysteine residues of said antibody or antibody moiety may e.g. be naturally occurring cysteine residues of the antibody such as those forming the interchain disulfide bonds following reduction of said cysteine residues for thiol-based conjugation, or said thiol groups of the cysteine residues said antibody may be genetically engineered, preferably at position D265 (according to EU numbering, D265C).

According to preferred embodiments, the pharmaceutical composition of the invention for use in the treatment of cancer as disclosed herein may e.g. be is used in the treatment of solid tumors, or a non-solid tumors. For example, the solid tumors are selected from the group comprising gastric carcinoma, adenocarcinoma, melanoma, ovarian carcinoma, uterine cancer, cervical cancer, breast cancer, including triple negative breast cancer, bronchial carcinoma, Ewing's sarcoma, liposarcoma, fibrosarcoma, leiomyosarcoma, thymoma, testicular cancer, neuroblastoma, glioma, prostate cancer, castration-resistant prostate cancer, gastrointestinal cancer, colorectal cancer, metastatic colorectal cancer (mCRC), stomach cancer, esophageal cancer, cancer of the larynx, cancer of the parotid, cancer of the biliary tract, rectal cancer, endometrial cancer, desmoid tumors, desmoplastic small round cell tumors, neuroectodermal tumors, retinoblastomas, rhabdomyosarcomas, Wilms tumors, osteosarcoma, chondrosarcoma, lung cancer, non-small cell carcinoma of the lung (NSCLC), alveolar rhabdomyosarcoma, Rhabdomyosarcoma, Askin's tumor, intra-abdominal desmoplastic-small cell tumor, Malignant papillary renal cell carcinoma, meningioma; small cell carcinoma of the lung, ependymoma, esthesioneuroblastoma (olfactory neuroblastoma), fibromatosis, ganglioglioma, Islet cell tumor, basal and squamous cell cancer, Large cell neuroendocrine carcinoma (LCNEC), Leydig cell tumor, salivary gland cancer, Pineoblastoma, Polymorphous low-grade adenocarcinoma, schwannoma, teratoma, thymoma. Non-solid tumors may e.g. be one of Hodgkin-lymphoma, follicular lymphoma, diffuse large B cell non-Hodgkin's lymphoma (DBNHL), subtypes of non-Hodgkin's lymphoma including mantle cell lymphoma (MCL), chronic lymphocytic leukemia (CLL), Richter syndrome, primary cutaneous marginal zone lymphoma (PCMZL), hairy cell leukemia, acute myeloid leukemia (AML), or multiple myeloma, Burkitt's lymphoma, anaplastic large-cell lymphoma, marginal zone B-cell lymphoma.

The term “cancer”, as used herein, shall refer to a general term for diseases in which abnormal cells divide without control. Cancer cells can invade nearby tissues and can spread through the bloodstream and lymphatic system to other parts of the body. There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas. A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

According to one embodiment, the cancer cells or the tumor as disclosed above are characterized by a hemizygous loss of TP53, POLR2A, or del(17p13). For example, the cancer cells or the tumor which may e.g. be treated with the pharmaceutical composition of the invention as disclosed herein, are characterized by a hemizygous loss of the POLR2A gene, or of the TP53 and POLR2A genes. The term “hemizygous” as used according to the invention refers to an individual or cell which has only one full allele of a gene or chromosome segment rather than the usual two. A hemizygote refers to a cell or organism whose genome includes only one full allele at a given locus, whether the allele is wildtype or mutant, e.g. the cells of any of the tumors or cancers disclosed above are hemizygotes for chromosome locus 17p13, preferably the cells of the cancers or tumors as disclosed above are hemizygotes for the genes TP53 and POLR2A. “TP53” as used herein refers “tumor protein 53” gene which encodes a tumor suppressor protein (P53) which comprises transcriptional activation, DNA binding, and oligomerization domains. The encoded protein responds to diverse cellular stresses to regulate expression of target genes, thereby inducing cell cycle arrest, apoptosis, senescence, DNA repair, or changes in metabolism. Mutations in this gene are associated with a variety of human cancers, including hereditary cancers such as Li-Fraumeni syndrome.

The tumour suppressor gene TP53 is frequently inactivated by mutation or deletion in a majority of human tumors. “POLR2A” as used herein refers to the POLR2A gene which encodes the largest subunit of the human RNA polymerase II complex and which is indispensable for the polymerase activity in mRNA synthesis. Hemizygous loss of chromosome 17p13, e.g. del(17p13.1), may be detected by fluorescence in situ hybridization (FISH) as disclosed in Merz et al. Am J Hematol. 2016 November;91(11):E473-E477.

The cells of the cancer types, or tumors as disclosed herein may e.g. not be a homogenous group of cells with regard to the loss of TP53 and/or POLR2A. For example, from about 1%, 2.5%, 5%, 7.5%, 10%, 15%, 20%, 25%, 30%, 40% 50%, 60% to about 70%, 75%, 80%, 85%, 90%, 95%, 100, or from about 70%, 75%, 80, 85% to about 90%, 92.5%, 95%, 97.5%, 100% of the cancer cells as disclosed above may be hemizygous for the del(17p13.1), TP53 and/or POLR2A, or e.g. at least 5%, 10%, 15%, 20%, 25%, 30%, 40%, 45%, 50%, 60%, 70%, 75%, 80%, 90%, 95% of the cancer cells as disclosed herein are hemizygous for del(17p13), or for TP53 and/or POLR2A. The use of the pharmaceutical composition of the invention as disclosed herein in the treatment of cancer according to the invention may e.g. be particularly advantageous on any of the tumors or cancers as disclosed above that are characterized by a hemizygous loss of chromosome 17p13.1, TP53 and/or POLR2A, because said tumors or cancer cells are at least 10-fold, 25-fold, 50-fold, 100-fold, 250-fold, 500-fold 1000-fold more sensitive to a cancer treatment using the pharmaceutical composition of the invention as disclosed herein. Accordingly, it may e.g. be beneficial to determine whether the cells oft he cancer as disclosed herein comprise or consist of cells which are hemizygous for the loss of TP53 and/or POLR2A, since at least 10-fold, 25-fold, 50-fold, 100-fold, 250-fold, 500-fold or 1000-fold less of the conjugate, pharmaceutical composition, or composition of the invention as disclosed herein may be used to achieve the desired therapeutic effect. Assays to assess the sensitivity of the cancer cells or tumors as disclosed above to a treatment with a pharmaceutical composition of the invention comprising a conjugate as disclosed herein can e.g. be done as described in Nature. 2015 Apr. 30; 520(7549): 697-701.

The pharmaceutical composition for use according to the invention as disclosed herein is preferably in liquid form, preferably in a formulation ready for subcutaneous administration to a patient in need thereof afflicted with cancer. According to some embodiments, the pharmaceutical composition for use according to the invention as disclosed herein further comprises one or more pharmaceutically acceptable buffers, surfactants, diluents, carriers, excipients, fillers, binders, lubricants, glidants, disintegrants, adsorbents, and/or preservatives.

For example, the pharmaceutical composition for use according to the invention may comprise at least one buffer to realize a pH of 4 to 8, preferably 5 to 7, more preferably of a pH of about 5.5 to about 6.5, or e.g. a pH of about 5, 5.5, 6, 6.5. Corresponding buffers may preferably one or more selected from the group consisting of malate, formate, citrate, acetate, propionate, pyridine, piperazine, cacodylate, succinate, 2-(N-morpholino)ethanesulfonic acid (MES), histidine, Tris, bis-Tris, phosphate, ethanolamine, carbonate, piperazine-N,N′-bis(2-ethanesulfonic acid) (PIPES), imidazole, BIS-TRIS propane, N,N-bis(2-hydroxyethyl)-2-aminoethanesulfonic acid (BES), 3-(N-morpholino) propanesulfonic acid) (MOPS), hydroxyethyl piperazine ethane sulfonic acid (HEPES), pyrophosphate, and triethanolamine, more preferably a histidine buffer, e.g., L-histidine/HCl, but is not limited thereto. The concentration of said at least one buffer may be in the range of 0.1 mM, 1 mM, 5 mM, 10 mM, 25 mM, 50 mM, 75 mM to about 100 mM, 125 mM, 150 mM, 175 mM, 200 mM, preferably 1 mM, 2 mM, 5 mM, 10 mM, 15 mM, 25 mM, 30 mM, 40 mM, 50 mM, more preferably of about 5 mM, 10 mM, 25 mM to about 50 mM.

For example, the pharmaceutical composition of the invention as disclosed herein may e.g. also comprise one or more stabilizers. The stabilizers in the pharmaceutical composition according to the present invention may be used without limitation, as long as they are commonly used in the art for the purpose of stabilizing proteins, and preferably, the stabilizers may be, for example, one or more selected from the group consisting of carbohydrates, sugars or hydrates thereof, sugar alcohols or hydrates thereof, and amino acids. For example, carbohydrates, sugars, or sugar alcohols used as stabilizer may be one or more selected from the group consisting of trehalose or hydrates thereof, sucrose, saccharin, glycerol, erythritol, threitol, xylitol, arabitol, ribitol, mannitol, sorbitol, galactitol, fucitol, iditol, inositol, volemitol, isomalt, maltitol, polyglycitol, cyclodextrin, hydroxylpropyl cyclodextrin, and glucose, but is not limited thereto. Said sugars or sugar alcohols used as stabilizers may be present in the pharmaceutical composition of the invention as disclosed herein in a concentration from about 0.1 mM, 1 mM, 2.5 mM, 5 mM, 10 mM, 20 mM, 25 mM, 50 mM, 75 mM, 100 mM to about 125 mM, 150 mM, 175 mM, 200 mM, 250 mM, 300 mM, 350 mM, 400 mM, 500 mM, preferably from about 10 mM, 25 mM, 50 mM, 100 mM, 125 mM to 150 mM, 200 mM, 250 mM, or e.g. at a concentration of 10 mM, 25 mM, 50 mM, 100 mM, 150 mM. Amino acids if present in the pharmaceutical composition of the invention may be one or more selected from the group consisting of glutamine, glutamic acid, glycine, lysine, lysilysine, leucine, methionine, valine, serine, selenomethionine, citrulline, arginine, asparagine, aspartic acid, ornithine, isoleucine, taurine, theanine, threonine, tryptophan, tyrosine, phenylalanine, proline, pyrrolysine, histidine, and alanine, but is not limited thereto. Said amino acids used as a stabilizer in the pharmaceutical composition according to the present invention may e.g. have a concentration of about 1 mM, 2.5 mM, 5 mM, 10 mM, 15 mM, 25 mM, 50 mM to about 75 mM, 100 mM, preferably of about 5 mM, 7.5 mM, 10 mM, 25 mM to about 30 mM, 40 mM, 50 mM.

For example, the pharmaceutical composition according to the invention may further comprise a non-ionic surfactant such as e.g. polyoxyethylene-sorbitan fatty acid ester (polysorbate or Tween), polyethylene-polypropylene glycol, polyoxyethylene-stearate, polyoxyethylene alkyl ethers, e.g., polyoxyethylene monolauryl ether, alkylphenyl polyoxyethylene ether [Triton-X], and a polyoxyethylene-polyoxypropylene copolymer [Poloxamer and Pluronic], and sodium dodecyl sulfate (SDS), polysorbate 20 or polysorbate 80, polyethylene glycol hexadecyl ether (Brij® 56); polyethylene glycol octadecyl ether (Brij® 72); polyoxyethylene 10 oleyl ether (Brij® 97); poloxamer 188, t-Octylphenoxypolyethoxyethanol (TRITON®×100); polyethylene glycol sorbitan monolaurate (TWEEN® 20); polyoxyethylenesorbitan monopalmitate (TWEEN® 40); polyethylene glycol sorbitan monostearate (TWEEN® 60); polyoxyethylenesorbitan Tristearate (TWEEN® 65); polyethylene glycol sorbitan monooleate (TWEEN 80); polyoxyethylenesorbitan Trioleate (TWEEN® 85); tris(hydroxymethyl) aminomethane lauryl sulfate (TRIZMA® dodecyl sulfate); block copolymer of polyethylene and polypropylene glycol (Pluronic® F68). Said non-ionic surfactant as disclosed above may e.g. be present in the pharmaceutical composition according to the present invention in the range of about 0.01 percent (w/v) to about 0.5% (w/v), preferably from about 0.1% (w/v), 0.2% (w/v), 0.3% (w/v) to about 0.4% (w/v), more preferably of about 0.1% (w/v) to 0.25% (w/v), 0.35% (w/v), 0.5% (w/v), whereby the expression “(w/v)” refers to weight per volume.

For example, the pharmaceutical composition for use according to the invention as disclosed herein may e.g. also comprise diluents selected from the group comprising of mannitol, microcrystalline cellulose, lactose, starch, dibasic calcium phosphate anhydrous, tribasic calcium phosphate, kaolin, sucrose, precipitated calcium carbonate, sorbitol, maltodextrin, powdered cellulose, micro crystalline cellulose and other materials known for such property. Said diluents may be present in the pharmaceutical composition for use according to the invention from about 0.1% (w/v), 0.25% (w/v), 0.5% (w/v), 1% (w/v), 2.5% (w/v), 5% (w/v), 7.5% (w/v), 10% (w/v), 12.5% (w/v), 15% (w/v), 20% (w/v), 25% (w/v), 30% (w/v) to about 35% (w/v), 40% (w/v), 45% (w/v), 50% (w/v), or from about 35% (w/v), 40% (w/v), 50% to about 60% (w/v), 75% (w/v), or from about 0.5% (w/v), 1% (w/v), 2.5% (w/v) to about 7.5% (w/v), 10% (w/v), 12.5% (w/v), 15% (w/v), 20% (w/v), 25% (w/v), 30% (w/v).

For example, the pharmaceutical composition for use according to the invention as disclosed herein may comprise lubricants that selected from the group comprising stearic acid, sodium stearyl fumarate, polyethylene glycol, magnesium stearate, calcium stearate, talc, zinc stearate, hydrogenated castor oil, silica, colloidal silica, cornstarch, calcium silicate, magnesium silicate, silicon hydrogel and other materials known for such property. Said lubricants may e.g. be present in the pharmaceutical composition for use according to the invention form about 0% (w/v), 0.1% (w/v), 0.25% (w/v), 0.5% (w/v), 0.75% (w/v), 1% (w/v) to about 1.25% (w/v), 1.5% (w/v), 1.75% (w/v), 2% (w/v), 2.5% (w/v), 3.0% (w/v), or from about 1.25% (w/v), 1.5% (w/v), 1.75% (w/v), 2% (w/v), 2.5% (w/v) to about 3% (w/v), 3.5% (w/v), 4% (w/v), 5% (w/v).

For example, the pharmaceutical composition for use according to the invention as disclosed herein may comprise binders selected from the group comprising polyvinylpyrrolidone, hydroxypropyl methylcellulose, acacia, alginic acid, hydroxy propyl cellulose, carboxymethylcellulose sodium, compressible sugar, ethylcellulose, gelatin, liquid glucose, methylcellulose, pregelatinized starch and other materials known to one of ordinary skill in the art. Binders in the dosage form ranges from 0% to 5.0% by weight.

For example, the pharmaceutical composition for use according to the invention as disclosed herein may comprise glidants selected from the group comprising colloidal silicon dioxide, colloidal silica, cornstarch, talc, calcium silicate, magnesium silicate, colloidal silicon, or silicon hydrogel. Said glidants may e.g. be present in said pharmaceutical composition in amounts ranging from about 0% (w/v), 0.1% (w/v), 0.25% (w/v), 0.5% (w/v), 0.75% (w/v), 1% (w/v) to about 1.25% (w/v), 1.5% (w/v), 1.75% (w/v), 2.0% by weight, or from about 1.25% (w/v), 1.5% (w/v), 1.75% (w/v) to about 2% (w/v).

The pharmaceutical composition of the invention may e.g. further comprise a preservative. A “preservative” according to the invention is a compound which can be added to the pharmaceutical composition disclosed herein to reduce bacterial growth. The addition of a preservative may, for example, facilitate the production of a multi-use or multiple-dosing of the pharmaceutical composition of the invention. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol in concentrations from about 0.9% (w/v) to about 2.0% (w/v), e.g. 0.9% (w/v), 1% (w/v), 1.25% (w/v), 1.5% (w/v), 1.75% (w/v), or 2% (w/v).

Typically, injectable volumes such as e.g. for the pharmaceutical composition of the invention, via the subcutaneous route are 3 ml or less, 2 ml or less, because of which it is desirable to use pharmaceutical compositions with high concentrations of the conjugate of the invention to be administered. Accordingly, it is desirable to use the pharmaceutical compositions according to the invention which comprises the conjugate of the invention as disclosed herein in a concentration from about 10 mg/ml, 20 mg/ml, 30 mg/ml, 40 mg/ml to about 50 mg/ml, 60 mg/ml, 75 mg/ml, 100 mg/ml, 125 mg/ml, 150 mg/ml, 175 mg/ml, 200 mg/ml, or from about 50 mg/ml, 60 mg/ml, 75 mg/ml, 100 mg/ml, 125 mg/ml to about 160 mg/ml, 180 mg/ml, 200 mg/ml, 250 mg/ml, 300 mg/ml, 350 mg/ml, 400 mg/ml, 450 mg/ml, 500 mg/ml or of about 10 mg/ml, 15 mg/ml, 20 mg/ml, 25 mg/ml, 30 mg/ml, 40 mg/ml, 50 mg/ml 75 mg/ml, or 100 mg/ml, preferably from about 25 mg/ml.

According to some embodiments, the amount of conjugate according to the invention injected in a volume of 3 ml or less, 2.5 ml or less, 2.0 ml or less, 1.5 ml or less of the pharmaceutical composition of the invention for use in the treatment of cancer as disclosed herein is selected from 10 mg, 15 mg, 20 mg, 25 mg, 30 mg, 40 mg, 50 mg, 75 mg, 100 mg, 125 mg, 150 mg, 175 mg, 200 mg, 250 mg, 300 mg, 350 mg, 400 mg, 450 mg or 500 mg (e.g. a fixed-dose administration) The effective amount of conjugate of the invention to be injected and correspondingly the amount and composition of the pharmaceutical composition of the invention to be injected may depend on the cancer or tumor type, cancer stage, overall health status of the patient to be treated, as well as body weight and size of the person. Fixed-dose subcutaneous administration of the pharmaceutical composition of the invention may e.g. be desirable if a combination treatment is intended, e.g. with standard of care treatments. Said Combination treatments may e.g. be desirable to reduce the risk of adverse reactions relative to higher dose monotherapy of the standard-of-care treatment or of a corresponding monotherapy using the subcutaneous administration of the pharmaceutical composition of the invention, (iii) may e.g. lower overall costs and (iv) improved medication concordance of a patient.

In one embodiment, the viscosity of the pharmaceutical composition of the invention is from about 5 cP, 10 cP, 15 cP, 20 cP, 30 cP, 40 cP, 50 cP, 60 cP to about 70 cP, 80 cP, 90 cP, 100 cP, or from about 10 cP, 15 cP, 20 cP to about 30 cP, 40 cP, 50 cP, 60 cP, preferably between 5 cP, 10 cP to about 25 cP, 30 cP. The term “viscosity” as used herein may be “kinematic viscosity” or “absolute viscosity.” “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. If one fluid takes 100 seconds to complete its flow and another fluid takes 200 seconds, the second fluid is twice as viscous as the first on a kinematic viscosity scale. “Absolute viscosity”, which is sometimes called dynamic or simple viscosity, is the product of kinematic viscosity and fluid density: Absolute Viscosity=Kinematic Viscosity×Density.

The dimension of kinematic viscosity is L²/T where L is a length and T is a time. Commonly, kinematic viscosity is expressed in centistokes (cSt). The SI unit of kinematic viscosity is mm²/s, which is 1 cSt. Absolute viscosity is expressed in units of centipoise (cP). The SI unit of absolute viscosity is the milliPascal-second (mPa·s), where 1 cP=1 mPa·s. The viscosity of the pharmaceutical composition of the invention may e.g. be determined using a commercially available glass capillary viscometers, or e.g. according to the methods disclosed in WO2016027859A1 the content of which is hereby incorporated by reference.

Pharmaceutical compositions which comprise antibodies or immunconjugates, such as the conjugates of the present invention, in concentrations that are sufficiently high to be able to administer subcutaneously an effective dose of the conjugate of the invention may have a viscosity greater than 20 cP, 30 cP, 40 cP, 50 cP. It may thus be advantageous to include one or more viscosity reducing agents to the pharmaceutical composition of the invention as disclosed herein.

Accordingly, the pharmaceutical composition of the invention as disclosed herein may optionally further comprise a viscosity reducing agent. For example, the pharmaceutical composition of the invention may comprise 1-butyl-3-methylimidazolium methanesulfonate (BMI Mes) and optionally one or more additional ionic liquids in an effective amount to significantly reduce the viscosity. Representative ionic liquids include 4-(3-butyl-1-imidazolio)-1-butane sulfonate (BIM), 1-butyl-3-methylimidazolium methanesulfonate (BMI Mes), 4-ethyl-4-methylmorpholinium methylcarbonate, (EMMC) and 1-butyl-1-methylpyrrolidinium chloride (BMP Chloride), at concentrations preferably between about 0.10 and about 0.50 M, equivalent to about 20-150 mg/mL as disclosed in EP3043774A1. Other viscosity reducing agents such as those disclosed in U.S. Pat. No. 9,605,051 B2 may be used.

In some embodiments, the pharmaceutical compositions according to the invention further comprise recombinantly produced human hyaluronidase such as e.g. ruHuPH20 (HYLENEX®). The use of such recombinant hyaluronidase in pharmaceutical compositions according to the invention may e.g. be advantageous if larger injection volumes of the pharmaceutical composition of the invention need to be administered subcutaneously to a patient in need thereof to administer a therapeutically effective dose of the conjugate of the invention, or in cases in which it is desirable to reduce the injection pressure that needs to be applied to effect the subcutaneous injection.

Thus, according to one embodiment, the pharmaceutical composition of the invention for use as disclosed herein may further comprise a soluble recombinant hyaluronidase, preferably a soluble recombinant human hyaluronidase, more preferably soluble recombinant human PH20. As used herein, “soluble recombinant human PH20 (rHuPH20)” refers to a composition: containing solubles form of human PH20 as recombinantly expressed and secreted in Chinese Hamster Ovary (CHO) cells as disclosed in e.g. WO 2004/078140 A2, or preferably variant forms of rHuPH20 as disclosed in WO2013/102144 A2 the content of both applications is hereby incorporated by reference.

The term “hyaluronidase” or “hyaluronidase activity” as used herein, refers to hyaluronidases which are a family of enzymes that catalyse the degradation of hyaluronic acid (HA). There are three main types of hyaluronidases: two classes of eukaryotic endoglycosidase hydrolases and a prokaryotic lyase-type of glycosidase. In humans, there are five functional hyaluronidases: HYAL1, HYAL2, HYAL3, HYAL4 and HYAL5 (also known as SPAM1 or PH-20); plus a pseudogene, HYAL6 (also known as HYALP1). The genes for HYAL1-3 are clustered in chromosome 3, while HYAL4-6 are clustered in chromosome 7. HYAL1 and HYAL2 are the major hyaluronidases in most tissues. GPI-anchored HYAL2 is responsible for cleaving high-molecular weight HA, which is mostly bound to the CD44 receptor. The resulting HA fragments of variable size are then further hydrolized by HYAL1 after being internalized into endo-lysosomes; this generates HA oligosaccharides.

According to their enzymatic mechanism, hyaluronidases are hyaluronoglucosidases (EC 3.2.1.35), i.e. they cleave the (1->4)-linkages between N-acetylglucosamine and glucuronate. The term hyaluronidase may also refer to hyaluronoglucuronidases (EC 3.2.1.36), which cleave (1->3)-linkages. In addition, bacterial hyaluronate lyases (EC 4.2.2.1) may also be referred to as hyaluronidases, although this is uncommon.

Mammalian-type hyaluronidases, (EC 3.2.1.35) which are endo-beta-N-acetylhexosaminidases with tetrasaccharides and hexasaccharides as the major end products. They have both hydrolytic and transglycosidase activities, and can degrade hyaluronan and chondroitin sulfates (CS), generally C4-S and C6-S.

Mammalian hyaluronidases can be further divided into two groups: neutral-active and acid-active enzymes. There are six hyaluronidase-like genes in the human genome, HYAL1, HYAL2, HYAL3, HYAL4, HYALP1 and PH20/SPAM1. HYALP1 is a pseudogene, and HYAL3 has not been shown to possess enzyme activity toward any known substrates. HYAL4 is a chondroitinase and exhibits little activity towards hyaluronan. HYAL1 is the prototypical acid-active enzyme and PH20 is the prototypical neutral-active enzyme. Acid-active hyaluronidases, such as HYAL1 and HYAL2 generally lack catalytic activity at neutral pH (i.e. pH 7). For example, HYAL1 has little catalytic activity in vitro over pH 4.5 (Frost et al Anal Biochemistry, 1997). HYAL2 is an acid-active enzyme with a very low specific activity in vitro. Recombinant human hyaluronidase PH20 (rHuPH20) was found to have optimal reaction rates in the pH range of 4.5-5.5 on HA substrates with sizes ranging from 90 to 752 kDa with rHuPH20 following Michaelis-Menten kinetics during the initial reaction time (see e.g. Anal Biochem. 2015 Jul. 1;480:74-81). Accordingly, the pH of the pharmaceutical composition for use according to the invention further comprising rHuPH20 should be chosen such that the activity of rHuPH20 is essentially optimal without negatively affecting the stability the conjugate of the pharmaceutical composition for use according to the invention as disclosed herein, if the pharmaceutical composition according to the invention comprises the rHuPH20. Accordingly, the pH of the pharmaceutical composition for use according to the invention may e.g. be from about pH 4.5, pH 4.6, pH4.7, pH 4.8, pH 4.9, pH5.0 to about pH5.5, pH6.0, pH6.2, pH6.5, or e.g. from about pH5.5, pH6.0 to about pH6.5, pH6.7, pH7.0. In case the pH the pharmaceutical composition of the invention requires a pH that results in hyaluronidase activity which corresponds to less than 90%, less than 80%, less than 70%, less than 60%, less than 50%, less than 40% of the activity of rHuPH20 at pH 4.5-pH5.5, the amount of rHuPH20 in the pharmaceutical composition for use according to the invention may be increased to offset the decrease in activity. Hyaluronidase activity of rHuPH20 may e.g. be determined as disclosed in WO 2013/102144, or e.g. as disclosed in Anal Biochem. 2015 Jul. 1;480:74-81.

The pharmaceutical composition for use according to the invention may e.g. comprise rHuPH20 from about 0.1 μg/ml, 0.25 μg/ml, 0.5 μg/ml, 1 μg/ml, 2.5 μg/ml, 5 μg/ml, 10 μg/ml, 15 μg/ml, 20 μg/ml, 25 μg/ml, 30 μg/ml, 35 μg/ml, 40 μg/ml, 50 μg/ml, 60 μg/ml, 75 μg/ml to about 80 μg/ml, 90 μg/ml, to 100 μg/ml, or from about 10 μg/ml, 15 μg/ml, 20 μg/ml, 25 μg/ml to about 30 μg/ml, 35 μg/ml, 40 μg/ml, 50 μg/ml.

For example, the pharmaceutical composition for use according to the invention may e.g. comprise from about 50 U/ml, 100 U/ml, 150 U/ml, 200 U/ml, 250 U/ml, 300 U/ml, 400 U/ml, 500 U/ml, 600 U/ml, 750 U/ml, 800 U/ml, 900 U/ml, 100 U/ml to about 1250 U/ml, 1500 U/ml, 1750 U/ml, 2000 U/ml, 2500 U/ml, 3000 U/ml, 3500 U/ml, 3750 U/ml, 4000 U/ml, 4500 U/ml, 5000 U/ml of hyaluronidase activity, or from about 1250 U/ml, 1500 U/ml, 1750 U/ml, 2000 U/ml, 2500 U/ml, 3000 U/ml, 3500 U/ml, 3750 U/ml, 4000 U/ml to about 5000 U/ml, 7500 U/ml, 10.000 U/ml hyaluronidase activity.

In some embodiments, the pharmaceutical composition for use according the invention may comprise a mixture of the conjugate of the invention in amounts as disclosed herein, preferably a therapeutically effective amount and rHuPH20 in amounts as disclosed above. Alternatively, the pharmaceutical composition for use according to the invention may comprise a first and a second component, wherein the first component comprises the hyaluronidase rHuPH20 in an amount as disclosed above, and a second component, which comprises the conjugate of the invention as disclosed herein. The first and second component of the pharmaceutical composition may e.g. be administered simultaneously, or sequentially. It is, however, preferred that the first and second component of said pharmaceutical composition are administer sequentially with the first component administered prior to the administration of the second component. Accordingly, the second component may be administered 1minute, 2 minutes, 3 minutes, 4 minutes, 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours within the administration of the first component, preferably, the second component may be administered 5 minutes, 15 minutes, 30 minutes, to an 1 hour within the administration of the first component. The first and second component of the pharmaceutical composition of the invention may preferably administered subcutaneously at the same injection site, or at injection sites 0.5 cm, 1 cm, 1.5 cm, 2 cm, 2.5 cm distant to each other.

In one embodiment, the subcutaneously injected volume of the pharmaceutical composition for use according to the invention as disclosed herein which comprise rHuPH20 as disclosed herein may e.g. be from about 0.5 ml, 1 ml, 2 ml, 2.5 ml, 3 ml, 3.5 ml, 4 ml, 4.5 ml, 5 ml to about 6 ml, 7 ml, 8 ml, 9 ml, 10 ml, or from about 6 ml, 7 ml, 8 ml, 9 ml to about 10 ml, or e.g. 0.75 ml, 1 ml, 1.25 ml, 1.5 ml, 2 ml, 2.5 ml, 3 ml, 3.5 ml, 4 ml, 4.5 ml, 5 ml, 5.5 ml, 6 ml, 6.5 ml, 7 ml, 7.5 ml, or 10 ml.

In some embodiments, the pharmaceutical composition for use according to the invention is co-administered with an immune checkpoint inhibitor. In the context of the present invention, the term “immune checkpoint inhibitor” or simply “checkpoint inhibitor” or “ICI” refers to any agent or compound that, either directly or indirectly, decreases the level of or inhibits the function of an immune checkpoint receptor protein or molecule found on the surface of an immune cell (for example, a T cell), or to any agent or compound that, either directly or indirectly, decreases the level of or inhibits the function of a ligand that binds to said immune checkpoint receptor protein or molecule, either as a soluble compound or on the surface of an immune cell-inhibitory cell. Such an inhibitory cell can be, for example, a cancer cell, a regulatory T cell, a tolerogenic antigen presenting cell, a myeloid-derived suppressor cells, a tumor-associated macrophage, or a cancer-associated fibroblast. Said ligand is typically capable of binding the immune checkpoint receptor protein or molecule on the immune cell. A non-limiting example of an immune checkpoint receptor protein-ligand pair is PD-1, PD-L1. PD-1 is an immune checkpoint receptor protein found on T-cells. PD-L1, which can be over-expressed by cancer cells, binds to PD-1 and helps the cancer cells to evade the host immune system attack. Accordingly, an immune checkpoint inhibitor prevents the PD-1/PD-L1 interaction by either blocking the PD-1 on the T cell (i.e. acts as a PD-I inhibitor) or the PD-L1 on the cancer cell (i.e., acts as a PD-L1 inhibitor), thereby maintaining or restoring anti-tumor T-cell activity or blocking inhibitory cancer cell activity.

Immune checkpoint inhibitors are thus antagonists of an immune inhibitory receptor, such PD-1, which inhibit, in this case, the PD-1 or PD-L1 in the PD-1/PD-L1 pathway. Examples of PD-1 or PD-L1 inhibitors include, without limitation, humanized or human antibodies antagonizing or blocking human PD-1 function such as pembrolizumab, pidilizumab, cemiplimab, JTX-4014, spartalizumab, sintilimab (IBI308), dostarlimab (TSR-042, WBP-285), INCMGA00012 (MGA012), AMP-224, PD1-1, PD1-2, PD1-3, PD1-4, PD1-5, BCD-100, AGEN-2034, Toripalimab (TAB001, JS001), or AMP-514 (MEDI0680), as well as fully human antibodies such as the PD-1 blocking nivolumab or blocking PD-L1 such as avelumab, durvalumab, cosibelimab (CK-301), WBP-3155 (CS1001), atezolizumab, envafolimab (KN035), or the recombinant anti-PD-L1 probody CX-072 (pacmilimab).

Pembrolizumab (formerly also known as lambrolizumab; trade name Keytruda; also known as MK-3475) disclosed e.g. in Hamid, O. et al. (2013) New England Journal of Medicine 369(2):134-44, is a humanized IgG4 monoclonal antibody that binds to PD-1; it contains a mutation at C228P designed to prevent Fc-mediated cytotoxicity. Pembrolizumab is e.g. disclosed in U.S. Pat. No. 8,354,509 and WO2009/114335. It is approved by the FDA for the treatment of patients suffering from unresectable or metastatic melanoma and patients with metastatic NSCLC.

Nivolumab (CAS Registry Number: 946414-94-4; BMS-936558 or MDX1106b) is a fully human IgG4 monoclonal antibody which specifically blocks PD-1, lacking detectable antibody-dependent cellular toxicity (ADCC). Nivolumab is e.g. disclosed in U.S. Pat. No. 8,008,449 and WO2006/121168. It has been approved by the FDA for the treatment of patients suffering from unresectable or metastatic melanoma, metastatic NSCLC and advanced renal cell carcinoma.

Pidilizumab (CT-011; Cure Tech) is a humanized IgG1k monoclonal antibody that binds to PD-1. Pidilizumab is e.g. disclosed in WO2009/101611.

PD1-1 to PD1-5 refer to anti-PD-1 antibodies as disclosed in WO2018/220169.

Ipilumumab (CAS Registry Number: 477202-00-9, which may also be referred to as 10D1, or MDX010, MDX-101) is a human IgG1 antibody that binds Cytotoxic T-lymphocyte antigen-4 (CTLA4). CTLA-4 is an inhibitory molecule that competes with the stimulatory CD28 for binding to B7 on antigen presenting cells. CTLA-4 and CD28 are both presented on the surface of T-cells. Ipilimumab is a human IgG1 that binds CTLA-4, preventing the inhibition of T-cell mediated immune responses to tumors. Ipilimumab is e.g. disclosed in WO 01/14424 as antibody “10D1”.

Envafolimab (CAS Registry Number: 2102192-68-5) is a subcutaneously (SC) administered single domain anti-programmed death ligand 1 (PD-L1) antibody as disclosed in U.S. Pat. No. 11,377,497 B2.

The INN designations for the antibodies disclosed herein as used herein are meant to also encompass all biosimilar antibodies of the corresponding originator antibody as disclosed herein, including but not limited to those biosimilar antibodies authorized under 42 USC § 262 subsection (k) in the US and equivalent regulations in other jurisdictions. The term “biosimilar” as used herein refers to antibodies that comprise an identical amino acid sequence compared to the antibody originally identified by the INN, however, biosimilar antibodies may differ in their glycosylation.

The pharmaceutical composition for use as disclosed herein may e.g. be co-administered with an immune checkpoint inhibitor in patients afflicted with a solid tumor, whereby the solid tumor is selected from the list of solid tumors disclosed herein.

The pharmaceutical composition for use according to the invention as disclosed herein may e.g. be co-administered with the subcutaneously administered pharmaceutical compositions of the invention. For example the pharmaceutical composition for use according to the invention may e.g. be administered prior to, simultaneously, or subsequent to the administration of the immune checkpoint inhibitor. If administered prior to the administration of the immune checkpoint inhibitor, the pharmaceutical composition as disclosed herein may be administered subcutaneously 5 minutes, 10 minutes, 15 minutes, 30 minutes, 1 hour, 1.5 hours, 2 hours, 4 hours, 6 hours, 8, hours, 12 hours, 16 hours, 18 hours, 24 hours, 48 hours prior to the administration of the immune checkpoint inhibitor. Alternatively, the immune checkpoint inhibitors as disclosed herein may, e.g. be administered 5 minutes, 10 minutes, 15 minutes, 30 minutes, 1 hour, 1.5 hours, 2 hours, 4 hours, 6 hours, 8, hours, 12 hours, 16 hours, 18 hours, 24 hours, 48 hours prior to the administration of the pharmaceutical composition of the invention. The pharmaceutical composition for use according to the invention and the immune checkpoint inhibitor as disclosed herein may in one alternative also be administered simultaneously, e.g. within 1 minute, 2 minutes, 3 minutes, 5 minutes, 10 minutes, 15 minutes apart from each other. Corresponding combinations of conjugates according to the invention and immune checkpoint inhibitors are e.g. disclosed in WO2022/096604 A1, the content of which is hereby incorporated in its entirety by reference.

In one embodiment, the pharmaceutical composition for use in the treatment of cancer according to the invention is administered subcutaneously for the treatment of any of the above cancer and tumor types. For example, said pharmaceutical composition may be injected subcutaneously using a suitable means for subcutaneous injection, such as a syringe or autoinjector as disclosed above. The pharmaceutical composition may e.g. be injected subcutaneously in the side or back of the upper arm, the abdomen or front of a patient's thighs depending on the volume to be injected and in consideration of the overall health status of the patient in need of a cancer treatment.

According to one embodiment, the pharmaceutical composition for use in the treatment of cancer is administered at least once, e.g. at least one dose, subcutaneously to a patient diagnosed with at least one type of cancer as disclosed herein.

According to one embodiment, the antibody of said conjugate of the invention specifically binds to a cell surface antigen on a tumor cell, such as a tumor-specific antigen, or to a tumor-associated antigen. The term “specifically binding” or any grammatical variation thereof as used herein, refers to the binding of a targeting moiety of the invention, such as e.g. the antibody or antibody moiety of the conjugates as disclosed herein, having a K_{d} of at least about 10⁻⁶M, 10⁻⁷ M, 10⁻⁸M, or from about 10⁻⁸M to about 10⁻⁹M, 10⁻¹⁰M, 10⁻¹¹M, 10⁻¹²M, or of about 5×10⁻⁹M, 5×10⁻¹⁰M to about 2.5×10⁻¹¹M, 5×10⁻¹¹M, 2.5×10⁻¹²M, 5×10⁻¹²M to its antigen. As used herein, a tumor-specific antigen is an antigenic substance produced in tumor cells, i.e., it triggers an immune response in the host. Tumor antigens are useful tumor markers in identifying tumor cells with diagnostic tests. Tumor antigens can e.g. be one of a glycan, a protein, a peptide expressed on the surface of a cancer cell and which is not expressed in healthy tissue or cells. The term “tumor-specific antigens” (TSA), as used herein may e.g. be one of a glycan, a protein, a peptide and are present only on tumor cells and not on any other cell. The term “Tumor-Associated Antigens” (TAA) refers to a glycan, a protein, or a peptide which is present on some tumor cells and also some normal, non cancerous cells.

According to one embodiment, the conjugate of the invention comprised in the pharmaceutical composition for use in the treatment of cancer as disclosed herein may target any suitable epitope on a target cell, such as e.g. an epitope on a cancer cell, whereby the cancer may e.g. be a cancer cell from any of the cancers disclosed herein. The term “epitope” as used herein refers to the part of a macromolecule, preferably a polypeptide, that is recognized by antigen-binding molecules, such as the antibody, or antigen-binding fragment thereof, or antigen-binding derivative thereof of the invention as disclosed herein, and more particularly by the antigen-binding site of said molecules. Epitopes define the minimum binding site for an antibody molecule, and thus represent the target of specificity of an antibody molecule. Epitopes can be further defined as structural epitopes or functional epitopes. A “structural epitope” consists of amino acids or other molecules in a region that is in close contact with the antibody usually revealed by a structure. A “functional epitope” is defined, as those parts of a molecule that make an energetic contribution to binding such that when they are changed there is a decrease in binding affinity. Therefore, the residues making contact with the paratope, and what residues that are contributing to the affinity, whether they are proximal or not are important considerations when defining the epitope. Structural epitopes may e.g. be a linear continuous sequence of about 5 amino acids to about 50, 100 amino acids in length, or a conformational epitope which is formed by the threedimensional structure of the polypeptide and which may comprise discontinuous amino acids of the polypeptide. Epitopes which may be specifically bound by the antibody of the conjugate of the invention may e.g. also be comprised on non-proteinaceous structures on cancer cells, such as e.g. glycans such as Lewis antigens (sialyl Lewis x (SLe^{x}) and sialyl Lewis a (SLe^{a})).

According to some embodiments, the antibody of said conjugate of the invention specifically binds to a cell surface antigen on a tumor cell, such as a tumor-specific antigen, or to a tumor-associated antigen selected from the group comprising EpCAM, HER2/neu, EGFR (HER1, ErbB1), TROP-2, BCMA, CD37, STEAP1, FXYD3, CA125, CD30, NCAM (CD56), MUC1, CEA (CD66e), VEGF, AFP, AXL, TYRO3, MER, CD20, CD19, CD52, CD268, CD28, CD80, CD22, CD4, CD2, CD33, CD30, CD38, CD52, CD80, CD140b, PSMA, TYR, FCRL2, MUC17, GPR143, NMNAT2, MAGE, MAGEC2, MAGE-A3, MART-1, WT-1, EPHA2, KRT19, CLDN7, DKK1, FGF19, SCN3A, SCN2A, GAS1, S100Z, GAPT, GPR35, NY-ESO, cadherin 24, DLK1, GPR173, ALK, GFRA3, GUCY2C, DLL3, PSMA, PROX1, PSCA, glypican-1, mesothelin, (prostate stem cell antigen), GAGE-1 (G Antigen 1), ganglioside/GD2, GnT-V, β1,6-N (acetylglucosaminyltransferase-V), UPAR (urokinase-type plasminogen activator receptor), sialyl Lewis x (SLe^{x}) and sialyl Lewis a (SLe^{a}).

According to a preferred embodiment, the pharmaceutical composition for use in the treatment of cancer as disclosed herein is administered subcutaneously and comprises a conjugate according to formula (I) as disclosed in WO2018/115466 A1, wherein the antibody “J22.9-ISY-D265C” designates an anti-BCMA antibody comprising a heavy chain amino acid sequence according to SEQ ID NO: 1 and a light chain amino acid sequence according to SEQ ID NO: 2 as disclosed in WO2018/115466, the content of which is hereby incorporated by reference.

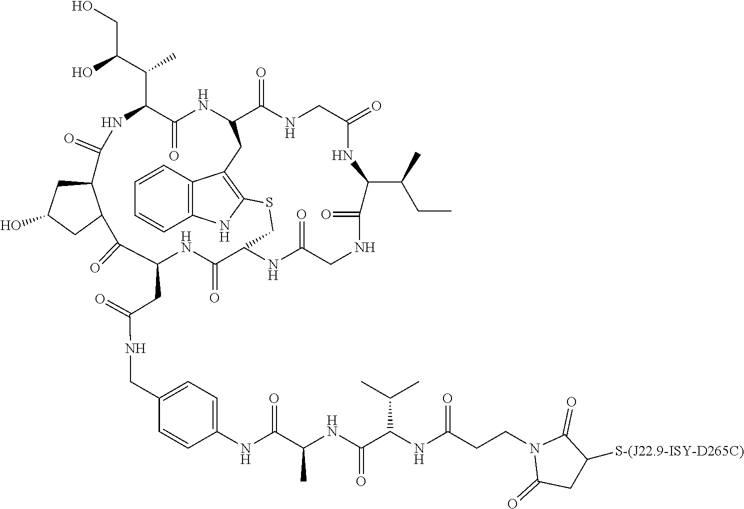

wherein the cancer is selected from the group comprising multiple myeloma, diffuse large B-cell lymphoma (DLBCL), and chronic lymphocytic leukemia (CLL), particularly multiple myeloma.

According to a preferred embodiment, the pharmaceutical composition for use in the treatment of cancer as disclosed herein is administered subcutaneously to patients afflicted with Relapsed Refractory Multiple Myeloma, wherein the patients are characterized by the following criteria:
Male or female aged≥18 years.
Life expectancy>12 weeks.
Eastern Cooperative Oncology Group Performance Status (PS) of 0 to 2.
A confirmed diagnosis of active MM according to the diagnostic criteria established by the International Myeloma Working Group (IMWG).
Must have undergone SCT or is considered transplant ineligible.
Must have undergone prior treatments with antimyeloma therapy which must have included an immunomodulatory drug, proteasome inhibitor, and anti-CD38 treatment, alone or in combination. In addition, the patient should either refractory or intolerant to any established standard of care therapy providing a meaningful clinical benefit for the patient assessed by the Investigator.
Measurable disease as per IMWG criteria.

Accordingly, the pharmaceutical composition of the invention for use in the treatment of Relapsed Refractory Multiple Myeloma are administered to a patient as disclosed herein.

Accordingly, the present pertains to a method of treating a patient afflicted with Relapsed Refractory Multiple Myeloma, whereby the patient is characterized by the above criteria.

According to a preferred embodiment, the pharmaceutical composition for use in the treatment of cancer as disclosed herein comprises a conjugate which comprises the anti-CD37 antibody chHH1-HDPLALA-D266C which comprises a heavy chain amino acid sequence according to SEQ ID NO: 11 and a light chain amino acid sequence according to SEQ ID NO: 12 as disclosed in WO 2022/194988 A2 (the content of which is hereby incorporated by reference), whereby the conjugate comprises one of the amatoxin-linkers moieties of formulae (XII), (XIII), (XIV), (XV), (XVI), (XVII), (XVIII), (XIX), (XX), (XXI) or (XXII) as disclosed herein, preferably (XII), (XIII), (XIV), (XV), (XVI), (XVII), (XX), or (XXII), more preferably (XII), (XIII), (XIV), (XVII), or (XX), particularly preferably (XII), (XIII), or (XIV).

According to a preferred embodiment, the pharmaceutical composition for use in the treatment of cancer as disclosed herein comprises a conjugate which comprises the anti-CD37 antibody chHH1-HDPLALA-D266C which comprises a heavy chain amino acid sequence according to SEQ ID NO: 11 and a light chain amino acid sequence according to SEQ ID NO: 12 as disclosed in WO 2022/194988 A2, whereby the conjugate comprises the amatoxin-linkers (XII).

According to a preferred embodiment, the pharmaceutical composition for use in the treatment of cancer as disclosed herein comprises a conjugate which comprises the anti-CD37 antibody chHH1-HDPLALA-D266C which comprises a heavy chain amino acid sequence according to SEQ ID NO: 11 and a light chain amino acid sequence according to SEQ ID NO: 12 as disclosed in WO 2022/194988 A2, whereby the conjugate comprises the amatoxin-linkers (XIII).

According to a preferred embodiment, the pharmaceutical composition for use in the treatment of cancer as disclosed herein comprises a conjugate which comprises the anti-CD37 antibody chHH1-HDPLALA-D266C which comprises a heavy chain amino acid sequence according to SEQ ID NO: 11 and a light chain amino acid sequence according to SEQ ID NO: 12 as disclosed in WO 2022/194988 A2, whereby the conjugate comprises the amatoxin-linkers (XIV).

According to a more preferred embodiment, the pharmaceutical composition for use in the treatment of cancer as disclosed herein comprises a conjugate which comprises the anti-CD37 antibody chHH1-HDPLALA-D266C which comprises a heavy chain amino acid sequence according to SEQ ID NO: 11 and a light chain amino acid sequence according to SEQ ID NO: 12 as disclosed in WO 2022/194988 A2, whereby the conjugate comprises the amatoxin-linkers (XII) and wherein the cancer is selected from the group comprising non-Hodgkin's lymphoma (NHL), follicular lymphoma, diffuse large B cell non-Hodgkin's lymphoma (DBNHL), subtypes of non-Hodgkin's lymphoma including mantle cell lymphoma (MCL), chronic lymphocytic leukaemia (CLL), Richter syndrome, primary cutaneous marginal zone lymphoma (PCMZL), hairy cell leukemia, acute myeloid leukemia (AML), rheumatoid arthritis, granulomatosis with polyangiitis and microscopic polyangiitis and pemphigus vulgaris, whereby said pharmaceutical composition is administered subcutaneously and may e.g. be formulated as disclosed herein.

According to a more preferred embodiment, the pharmaceutical composition for use in the treatment of cancer as disclosed herein comprises a conjugate which comprises the anti-CD37 antibody chHH1-HDPLALA-D266C which comprises a heavy chain amino acid sequence according to SEQ ID NO: 11 and a light chain amino acid sequence according to SEQ ID NO: 12 as disclosed in WO 2022/194988 A2, whereby the conjugate comprises the amatoxin-linkers (XIII) and wherein the cancer is selected from the group comprising non-Hodgkin's lymphoma (NHL), follicular lymphoma, diffuse large B cell non-Hodgkin's lymphoma (DBNHL), subtypes of non-Hodgkin's lymphoma including mantle cell lymphoma (MCL), chronic lymphocytic leukemia (CLL), Richter syndrome, primary cutaneous marginal zone lymphoma (PCMZL), hairy cell leukemia, acute myeloid leukemia (AML), rheumatoid arthritis, granulomatosis with polyangiitis and microscopic polyangiitis and pemphigus vulgaris.

According to a more preferred embodiment, the pharmaceutical composition for use in the treatment of cancer as disclosed herein comprises a conjugate which comprises the anti-CD37 antibody chHH1-HDPLALA-D266C which comprises a heavy chain amino acid sequence according to SEQ ID NO: 11 and a light chain amino acid sequence according to SEQ ID NO: 12 as disclosed in WO 2022/194988 A2, whereby the conjugate comprises the amatoxin-linkers (XIV) and wherein the cancer is selected from the group comprising non-Hodgkin's lymphoma (NHL), follicular lymphoma, diffuse large B cell non-Hodgkin's lymphoma (DBNHL), subtypes of non-Hodgkin's lymphoma including mantle cell lymphoma (MCL), chronic lymphocytic leukaemia (CLL), Richter syndrome, primary cutaneous marginal zone lymphoma (PCMZL), hairy cell leukemia, acute myeloid leukemia (AML), rheumatoid arthritis, granulomatosis with polyangiitis and microscopic polyangiitis and pemphigus vulgaris.

According to a preferred embodiment, the pharmaceutical composition for use in the treatment of cancer as disclosed herein comprises a conjugate which comprises an anti-PSMA antibody as disclosed in WO 2020/025564 (the content of which is hereby incorporated by reference) selected from the group comprising 3-F11-var1, 3-F11-var2, 3-F11-var3, 3-F11-var4, 3-F11-var5, 3-F11-var6, 3-F11-var7, 3-F11-var8, 3-F11-var9, 3-F11-var10, 3-F11-var11, 3-F11-var12, 3-F11-var13, 3-F11-var14, 3-F11-var115, or 3-F11-var16 conjugated to amatoxin-linkers moieties according to formulae (XII), (XIII), (XIV), (XV), (XVI), (XVII), (XVIII), (XIX), (XX), (XXI) or (XXII) as disclosed herein, preferably (XII), (XIII), (XIV), (XV), (XVI), (XVII), (XX), or (XXII), more preferably (XII), (XIII), (XIV), (XVII), (XX), most preferred the antibody is one of 3-F11-var1, 3-F11-var13, or 3-F11-var16 conjugated to an amatoxin-linker moiety according to any of formulae (XII), (XIII), (XIV), (XVII), or (XX):

| Antibody (as disclosed | |
|---|---|
| in WO 2020/025564) | Amatoxin-linker |
| 3-F11-var1 | (XII), (XIII), (XIV), (XVII), or (XX) |
| 3-F11-var13 | (XII), (XIII), (XIV), (XVII), or (XX) |
| 3-F11-var16 | (XII), (XIII), (XIV), (XVII), or (XX) |

According to a preferred embodiment, the pharmaceutical composition for use in the treatment of cancer as disclosed herein comprises a conjugate which comprises the anti-PSMA antibody 3-F11-var1 as disclosed in WO 2020/025564 conjugated to the amatoxin linker moiety (XII).

According to a preferred embodiment, the pharmaceutical composition for use in the treatment of cancer as disclosed herein comprises a conjugate which comprises the anti-PSMA antibody 3-F11-var1 as disclosed in WO 2020/025564 conjugated to the amatoxin linker moiety (XIII).

According to a preferred embodiment, the pharmaceutical composition for use in the treatment of cancer as disclosed herein comprises a conjugate which comprises the anti-PSMA antibody 3-F11-var1 as disclosed in WO 2020/025564 conjugated to the amatoxin linker moiety (XIV).

According to a preferred embodiment, the pharmaceutical composition for use in the treatment of cancer as disclosed herein comprises a conjugate which comprises the anti-PSMA antibody 3-F11-var13 as disclosed in WO 2020/025564 conjugated to the amatoxin linker moiety (XII).

According to a preferred embodiment, the pharmaceutical composition for use in the treatment of cancer as disclosed herein comprises a conjugate which comprises the anti-PSMA antibody 3-F11-var13 as disclosed in WO 2020/025564 conjugated to the amatoxin linker moiety (XIII).

According to a preferred embodiment, the pharmaceutical composition for use in the treatment of cancer as disclosed herein comprises a conjugate which comprises the anti-PSMA antibody 3-F11-var16 as disclosed in WO 2020/025564 conjugated to the amatoxin linker moiety (XIV).

According to a preferred embodiment, the pharmaceutical composition for in the treatment of cancer as disclosed herein comprises a conjugate which comprises the anti-PSMA antibody 3-F11-var16 as disclosed in WO 2020/025564 conjugated to the amatoxin linker moiety (XII).

According to a preferred embodiment, the pharmaceutical composition for use in the treatment of cancer as disclosed herein comprises a conjugate which comprises the anti-PSMA antibody 3-F11-var16 as disclosed in WO 2020/025564 conjugated to the amatoxin linker moiety (XIII).

According to a preferred embodiment, the pharmaceutical composition for use in the treatment of cancer as disclosed herein comprises a conjugate which comprises the anti-PSMA antibody 3-F11-var16 as disclosed in WO 2020/025564 conjugated to the amatoxin linker moiety (XIV).

According to a preferred embodiment, the pharmaceutical composition for use in the treatment of cancer as disclosed herein comprises a conjugate which comprises an anti-GCC antibody as disclosed in PCT/EP2023/080350 (the content of which is hereby incorporated by reference) selected from the group comprising antibody mAb1, mAb8, mAb, mAb41 each comprising the comprising the mutations L234A, L235A and D265C (numbering according to EU numbering system) conjugated to amatoxin-linkers moieties according to formulae (XII), (XIII), (XIV), (XV), (XVI), (XVII), (XVIII), (XIX), (XX), (XXI) or (XXII) as disclosed herein, preferably (XII), (XIII), (XIV), (XV), (XVI), (XVII), (XX), or (XXII), more preferably (XII), (XIII), (XIV), (XVII), (XX), most preferred (XII), or (XIV).

| Antibody (as disclosed in | |
|---|---|
| PCT/EP2023/080350) | Amatoxin-linker |
| mAb1 comprising | (XII), (XIII), (XIV), (XVII), or (XX) |
| SEQ ID NO: 80, 78 | |
| mAb8 comprising | (XII), (XIII), (XIV), (XVII), or (XX) |
| SEQ ID NO: 87, 85 | |
| mAb41 comprising | (XII), (XIII), (XIV), (XVII), or (XX) |
| SEQ ID NO: 113, 115 | |

The term “GCC” refers to Guanylyl Cyclase C (GUCY2C, EC: 4.6.1.2), preferably human GCC, which is a member of a family of receptor-enzyme proteins synthesizing guanosine 3′,5′-cyclic monophosphate (cyclic GMP; cGMP). GUCY2C is a transmembrane receptor for the endogenous hormonal ligands: guanylin and uroguanylin. Ligand binding to the extracellular receptor catalyzes the conversion of GTP into cyclic GMP (cGMP) and initiates downstream cGMP-related signaling pathways, which are implicated in the regulation of intestinal homeostatic processes such as epithelial cell proliferation, differentiation, and apoptosis.

According to one embodiment, the pharmaceutical composition for use in the treatment of cancer according to the invention as disclosed herein is administered to said patient multiple times e.g. twice, or three times, or e.g. periodically every 30 days, 60 days, 90 days, 120 days, 180 days, 360 days, or from about every 21 days, 28 days, to about 35 days, 36 days, 37 days, 38 days, 39 days, 40 days, 41 days, 42 days, or every 22 days, 23 days, 24 days, 25 days, 26 days, 27 days.

According to one embodiment, the pharmaceutical composition for use in the treatment of cancer according to the invention as disclosed herein increases the maximum tolerated dose (MTD) by at least 30%, 40%, 50% compared to the MTD of the pharmaceutical composition when administered intravenously. The term “maximum tolerated dose” (MTD) as used in the present invention refers to the highest tolerated dose of a drug, such as the conjugates of the invention disclosed herein, that can be administered to animals without causing severe toxicity or mortality. Accordingly, the pharmaceutical composition for use in the treatment of cancer, according to the invention is characterized by a greater therapeutic index (TI), than the a corresponding pharmaceutical composition for use in the treatment of cancer and which is administered to e.g. a mammal, e.g. a patient afflicted with cancer, via the intravenous (i.v.) route. The term “therapeutic index” as used herein refers to the ratio that compares the blood concentration at which a drug becomes toxic and the concentration at which the drug is effective. The larger the therapeutic index (TI), the safer the drug is. The TI may be e.g. calculated as: TI=MTD/MED, whereby the term “MED” refers to minimum effective dose, which is the lower bound for therapeutically effective doses.

According to one embodiment, the present invention pertains to the use of a conjugate comprising an amatoxin linker moiety according to any of formulae (I), (II), (III), (IV), (V), (VI), (VII), (VIII), (IX), (X), or (XI), or conjugates according to any of formulae (XII), (XIII), (XIV), (XV), (XVI), (XVII), (XVIII), (XIX), (XX), (XXI) or (XXII) in the manufacture of a pharmaceutical composition for use in the treatment of cancer according to the invention as disclosed herein, wherein the pharmaceutical composition is administered subcutaneously.

According to one embodiment, the present invention pertains to an immune checkpoint inhibitor selected from the group of avelumab, nivolumab, pembrolizumab, ipilimumab, or durvalumab, envafolimab for use in the manufacture of a pharmaceutical composition according to the invention as disclosed herein.

According to one aspect, the present invention pertains to method of treating a patient afflicted with cancer, wherein the method comprises administering subcutaneously to said patient an effective dose of the pharmaceutical composition as disclosed herein. The term “effective dose” as used herein refers to an amount of the pharmaceutical composition as disclosed herein, or of a conjugate as disclosed herein that produces one or more desired responses in the subject, for example, the killing of cancer cells that express the cell surface antigen, or tumor-associated, or tumor specific antigen specifically bound by the antibody of the conjugate.

According to some embodiments, the method of treating a patient as disclosed herein comprises administering the pharmaceutical composition of the invention to said patient at a single injection site, or to multiple injection sites. Multiple injection sites may include two or more injection sites at which the pharmaceutical composition of the invention as disclosed herein is administered subcutaneously. Depending on the composition of the pharmaceutical composition of the invention and the overall health status of the patient such as for example the amount of subcutaneous fat of a respective patient the pharmaceutical composition of the invention may be administered at a single injection site, or multiple injection sites.

Pharmaceutical composition of the invention which comprise two components, e.g. conjugate according to the invention as disclosed herein and a second component such as e.g. rHuPh20, may require injecting the first and second component in spatial proximity, e.g. within 0.5 cm to about 3 cm apart from each other. Said first and second component may e.g. be administered subcutaneously to said patient as disclosed herein, e.g. the first component may be administered prior to, simultaneously with, or subsequent to the administration of the second component. Alternatively, the second component (rHuPH20) may e.g. be administered prior to the administration of the first component as disclosed herein, or simultaneously with the first component, or subsequent to the first component, however, it is preferred that the second component is administered prior to the administration of the first component, e.g. from about 1 minute, 2 minutes, 3 minutes, 4 minutes, 5 minutes, 6 minutes, 7 minutes, 8 minutes, 9 minutes, 10 minutes to about 15 minutes, 20 minutes, 25 minutes, 30 minutes, 35 minutes, 40 minutes, 45 minutes, 60 minutes, or e.g. from about 15 minutes, 20 minutes, 25 minutes, 30 minutes to about 90 minutes prior to the administration of the first component For example, the first, or second component of the pharmaceutical composition of the invention may be administered subcutaneously within 1minute, 2 minutes, 3 minutes, 4 minutes, 5 minutes, 15 minutes, 30 minutes, 1 hour, 2 hours, 3 hours, 4 hours, 6 hours, 8 hours, 12 hours, 16 hours, 18 hours, 20 hours, 22 hours, 24 hours, 30 hours, 36 hours, 40 hours or 48 hours after subcutaneous administration of the first, or second component. Corresponding treatments may e.g. be repeated periodically as may be medically required, e.g. every 21 days, 28 days, 35 days, 42 days, 60 days, 90 days, 120 days, 180 days, 360 days, for instance two time, three times, or four times.

In some embodiments, the method of treating a patient according to the invention comprises administering to said patient subcutaneously about 0.5 ml, 0.75 ml, 1 ml, 1.5 ml, 2 ml, 2.5 ml, 3 ml, 3.5 ml to about 4 ml, 5 ml, 6 ml, 7 ml, 8 ml, 9 ml, or 10 ml, or e.g. about 1.25 ml, 1.75 ml, 2.25 ml to about 2.5 ml, 3 ml, 3.5 ml of the pharmaceutical composition of the invention whereby the volume to be injected may e.g. depend on the use of rHuPH20 to facilitate the subcutaneous injection of the pharmaceutical composition of the invention as disclosed herein.

The effective dose of the pharmaceutical composition according to the invention to be administered subcutaneously to said patient may vary depending on multiple factors, such as cancer type, cancer stage, age of the patient, or body weight, or body surface of said patient. In some embodiments the effective dose of said pharmaceutical composition of the invention administered to said patient subcutaneously is from about 60 μg/kg, 75 μg/kg 100 μg/kg, 125 μg/kg, 150 μg/kg, 200 μg/kg body weight (b.w.) to about 250 μg/kg, 300 μg/kg, 350 μg/kg, 400 μg/kg, 450 μg/kg, 500 μg/kg, 600 μg/kg, 700 μg/kg, 750 μg/kg, 800 μg/kg, 900 μg/kg, 1 mg/kg, 1.25 mg/kg, 1.5 mg/kg, 1.75 mg/kg, 2 mg/kg body weight, or from about 175 μg/kg, 250 μg/kg, 275 μg/kg 300 μg/kg, 350 μg/kg, 475 μg/kg, 550 μg/kg, 75 μg/kg, 850 μg/kg, 1 mg/kg, 1.25 mg/kg, 1.5 mg/kg body weight to about 375 μg/kg, 425 μg/kg, 475 μg/kg, 950 μg/kg, 2.5 mg/kg, 3 mg/kg body weight. If the body surface of said patient is used to determine the effective dose, the body surface area (BSA) may be calculated according to the Du Bois formula:

BSA=0.007184×W^{0.425}×H^{0.725}, whereby W is the body mass in kg, and H the patient's height in cm.

According to one embodiment, the invention pertains to a method of treating a patient afflicted with cancer, whereby the method comprises administering to said patient the pharmaceutical composition of the invention subcutaneously as disclosed herein, wherein the patient has failed prior standard-of-care first-line, second-line, or third-line cancer treatment for the respective cancer.

In one embodiment the present invention pertains to a method of delivering an amatoxin-linker payload as disclosed herein (e.g. according to one of formulae (I), (II), (III), (IV), (V), (VI), (VII), (VIII), (IX), (X), or (XI) as disclosed herein) to a cell, whereby the method comprises administering subcutaneously a pharmaceutical composition comprising said conjugate of the invention to a patient in need thereof which specifically binds to an epitope on said cell.

Sequence Listing

| SEQ ID | | |
|---|---|---|
| NO | SEQUENCE | Description |
| 1 | QVQLVQSGAEVKKPGASVKVSCKASGYIFISS | SEQ ID NO: 80 of |
| | AMHWVRQAPGQRLEWMGLINPGNGNTKYSQKF | PCT/EP2023/080350 |
| | QGRVTITRDTSASTAYMELSSLRSEDTAVYYC | mAb1 heavy chain (HC) |
| | ARAYFRGLGFDIWGQGTLVTVSSASTKGPSVF | comprising mAb1 heavy |
| | PLAPSSKSTSGGTAALGCLVKDYFPEPVTVSW | chain (HC), comprising |
| | NSGALTSGVHTFPAVLQSSGLYSLSSVVTVPS | the mutations L234A, |
| | SSLGTQTYICNVNHKPSNTKVDKKVEPKSCDK | L235A and D265C |
| | THTCPPCPAPEAAGGPSVFLFPPKPKDTLMIS | (numbering according |
| | RTPEVTCVVVCVSHEDPEVKFNWYVDGVEVHN | to EU numbering |
| | AKTKPREEQYNSTYRVVSVLTVLHQDWLNGKE | system) |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 2 | DIQMTQSPSSLSASAGDRVTITCRASQSISSY | SEQ ID NO: 78 of |
| | LNWYQQKPGKAPKLLIYAASSLQSGVPSRFSG | PCT/EP2023/080350 |
| | SGSGTDFTLAISSLQPEDFATYYCQQSYSTPW | mAb1 light chain (LC) |
| | TFGQGTKVDIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| 3 | DIRMTQSPSSLSASAGDRVTITCRASQSISSY | SEQ ID NO: 85 of |
| | LNWYQQKPGKAPKLLIYAASSLQSGVPSRFSG | PCT/EP2023/080350 |
| | SGSGTDFTLTISSLQPEDFATYYCQQSYSTPL | mAb8 LC |
| | TFGGGTKLEIKRTVAAPSVFIFPPSDEQLKSG | |
| | TASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| 4 | QVQLVQSGAEVKKPGASVKVSCKASGYIFISS | SEQ ID NO: 87 of |
| | AMHWVRQAPGQRLEWMGLINPGNGNTKYSQKF | PCT/EP2023/080350 |
| | QGRVTITRDTSASTAYMELSSLRSEDTAVYYC | mAb8 HC comprising the |
| | ARAYFRGLGFDIWGQGTLVTVSSASTKGPSVF | mutations L234A, L235A |
| | PLAPSSKSTSGGTAALGCLVKDYFPEPVTVSW | and D265C (numbering |
| | NSGALTSGVHTFPAVLQSSGLYSLSSVVTVPS | according to EU |
| | SSLGTQTYICNVNHKPSNTKVDKKVEPKSCDK | numbering system) |
| | THTCPPCPAPEAAGGPSVFLFPPKPKDTLMIS | |
| | RTPEVTCVVVCVSHEDPEVKFNWYVDGVEVHN | |
| | AKTKPREEQYNSTYRVVSVLTVLHQDWLNGKE | |
| | YKCKVSNKALPAPIEKTISKAKGQPREPQVYT | |
| | LPPSREEMTKNQVSLTCLVKGFYPSDIAVEWE | |
| | SNGQPENNYKTTPPVLDSDGSFFLYSKLTVDK | |
| | SRWQQGNVFSCSVMHEALHNHYTQKSLSLSPG | |
| | K | |
| 5 | QSALTQPASVSGSPGQSITISCTGTSSDVGSY | SEQ ID NO: 113 of |
| | NLVSWYQQHPGKAPKLMIYEGSKRPSGVSNRF | PCT/EP2023/080350 |
| | SASKSGNTASLTISGLQTEDEADYYCSSYVPR | mAb41 LC |
| | SSLVFGGGTKLTVLGQPKAAPSVTLFPPSSEE | |
| | LQANKATLVCLISDFYPGAVTVAWKADSSPVK | |
| | AGVETTTPSKQSNNKYAASSYLSLTPEQWKSH | |
| | RSYSCQVTHEGSTVEKTVAPTECS | |
| 6 | QVQLVQSGAEVKKPGSSVKVSCKASGGTFSTY | SEQ ID NO: 115 of |
| | TINWVRQAPGQGLEWMGRIIPVLGIANYAQKF | PCT/EP2023/080350 |
| | QGRVTITADKSTSTAYMELSSLRSEDTAVYYC | mAb41 HC comprising |
| | ARDTPRLRSSYYMDVWGQGTLVTVSSASTKGP | the mutations L234A, |
| | SVFPLAPSSKSTSGGTAALGCLVKDYFPEPVT | L235A and D265C |
| | VSWNSGALTSGVHTFPAVLQSSGLYSLSSVVT | (numbering according |
| | VPSSSLGTQTYICNVNHKPSNTKVDKKVEPKS | to EU numbering |
| | CDKTHTCPPCPAPEAAGGPSVFLFPPKPKDTL | system) |
| | MISRTPEVTCVVVCVSHEDPEVKFNWYVDGVE | |
| | VHNAKTKPREEQYNSTYRVVSVLTVLHQDWLN | |
| | GKEYKCKVSNKALPAPIEKTISKAKGQPREPQ | |
| | VYTLPPSREEMTKNQVSLTCLVKGFYPSDIAV | |
| | EWESNGQPENNYKTTPPVLDSDGSFFLYSKLT | |
| | VDKSRWQQGNVFSCSVMHEALHNHYTQKSLSL | |
| | SPGK | |

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Example 1: Conjugation of Amatoxin-Linker Payloads

Antibodies were conjugated to the amatoxin linker conjugates by means of the so-called THIOMAB® technology which utilizes cysteine-engineered antibodies. In this approach, the conjugation is mediated by conjugating the maleimide residue of the toxin linker construct to the free SH group of an engineered cysteine residue in the antibody which has been introduced by e.g. site-directed mutagenesis. The conjugation is shown in the following reaction scheme:

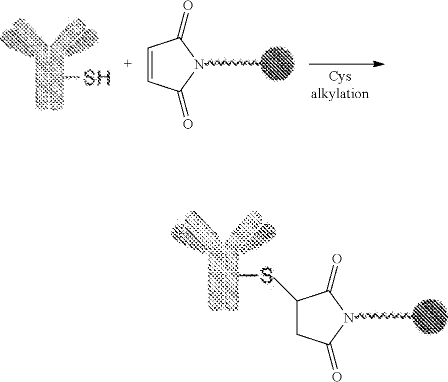

The principles of this conjugation method, are disclosed in Junutula J R et al. (2008). Nat Biotechnology Vol. 26: 925-932, the content of which is incorporated herein by reference. In brief: Prior to conjugation of the cysteine-engineered antibody (which may also be referred to as a THIOMAB®) to an amatoxin linker payload as disclosed herein which comprise a maleimide-containing linker, the blocking cysteine or glutathione that was present on the introduced cysteine may e.g. be removed by mild reduction in PBS at 25° C. by the addition of 10-40 fold molar excess reducing agent, TCEP or dithiothreitol (DTT) followed by diafiltration. To re-form the interchain disulfide bonds, the cysteine-engineered antibody may e.g. incubated for three hours at 25° C. with CuSO₄ or with dhAA (Sigma-Aldrich) at a 2-20-fold molar excess over the reducing agent concentration. The formation of interchain disulfide bonds may e.g. be monitored either by nonreducing SDS-PAGE or by denaturing reversed phase high-performance liquid chromatography (HPLC) PLRP column chromatography. The conjugation of the maleimide-linker amatoxin payload to the cysteine-engineered antibody may e.g. be done using 2-4 molar equivalents of the amatoxin linker payload, quenching may e.g. be done using 20-25 molar equivalents of N-acetyl-L-cysteine followed by an incubation at 25° C. for about 1 hour. The antibody conjugate may then e.g. be purified on HiTrap® S column (GE Healthcare Bio-Sciences) to remove excess reagents, optionally preceded by a quenching step using N-acetyl-L-cysteine for 15 minutes.

The antibodies used in the present experiments comprise a D265C substitution in both Fc domains, in order to provide a cystein residue that has such free SH group. The respective technology is disclosed in WO2016/142049 A1, the content of which is incorporated herein by reference, and which results in a homogenous product with a fixed drug to antibody ration (“DAR”) of 2 and a site-specific conjugation.

### Example 2: Murine Prostate Cancer Model

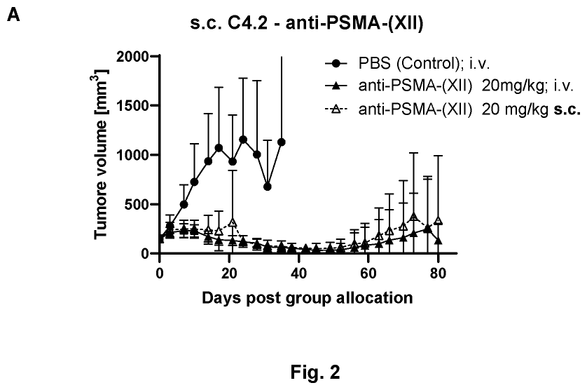

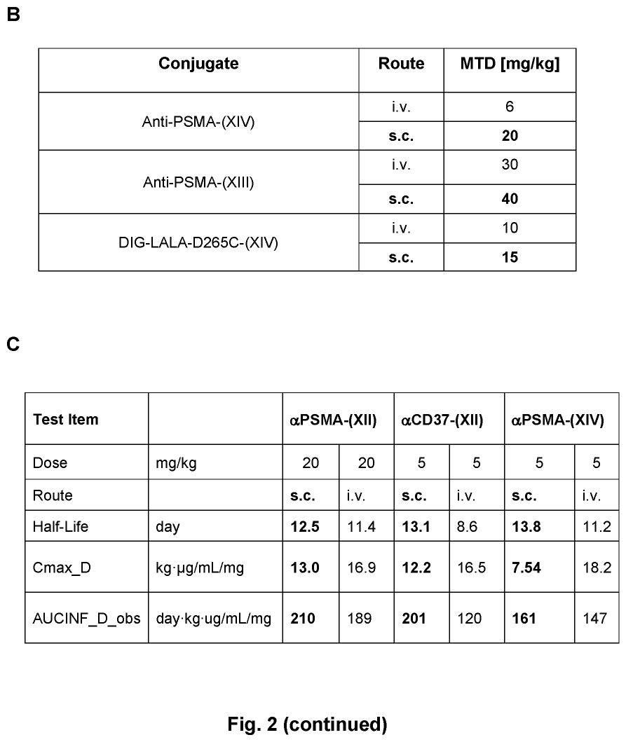

Male CB17 Scid animals were inoculated subcutaneously with 2.5×10⁶ C4-2 tumor cells in 200 μL RPMI w/o PR containing 50% GFR Matrigel into their right flanks. Once a mean tumor volume of about 140 mm³ (preferred range 80-200 mm³) was reached, animals were randomized according to tumor sizes (n=10). Therapy was initiated one day after group allocation. Mice were treated with a single dose PBS or 20 mg/kg of an anti-PSMA conjugate comprising an anti-PSMA antibody as disclosed in WO2020/025564A1 conjugated to the amatoxin-linker moiety (XII), (2.5 ml/kg) i.v. or s.c. (between the shoulders). Tumor volumes were measured twice per week by calliper measurements and body weight was determined in parallel. Animals were sacrificed and a necropsy performed, when either tumor volumes was >1600 mm³ or mice needed to be sacrificed for ethical reasons. The results are shown in FIG. 2A demonstrating that the single dose s.c. injection (open triangles) of the anti-PSMA conjugate was equally effective than a single dose i.v. (filled triangles).

### Example 3: Murine Chronic B Cell Leukemia Cancer Model

Female CB17 Scid animals were inoculated intravenously with 2.5×10⁶ MEC-2 (human CLL) tumor cells in 200 μL PBS on day −3. Therapy was initiated three days post tumor cell inoculation (n=10). Mice were treated with PBS or the anti-CD37 conjugate chHH1-HDPLALA-D266C conjugated to the amatoxin-linker moiety (XII) either i.v. or s.c. (10 ml/kg). Body weight was determinate twice per week. Clinical signs and survival were monitored and recorded daily. Animals were sacrificed, and a necropsy performed, when mice needed to be sacrificed for ethical reasons. The results are shown in FIG. 2B demonstrating that a single dose of the conjugate given subcutaneously at a dose of 1 mg/kg (open triangle), or 3 mg/kg (open square) were equally effective in prolonging the probability of survival compared to a corresponding treatment in which the conjugate was administered i.v. at the same dose of 1 mg/kg (filled triangle) and 3 mg/kg (filled square).

### Example 4: Anti-Tumor Efficacy of Anti-CD37-(XII) Conjugates in a Raji-Luc (Human Burkitt's Lymphoma) Disseminated Xenograft Model in Female CB17-SCID Mice

The aim of this study was to evaluate the anti-tumor efficacy of anti-CD37-(XII) conjugate after single subcutaneous dose in a Raji-Luc (human Burkitt's lymphoma) disseminated xenograft model in female CB17-SCID mice.

### Study Design

20 female CB17-SCID mice were allocated to 2 groups with 10 animals each and inoculated intravenously with 2.5×10⁶ Raji-Luc tumor cells (in 200 μl RPMI without Phenol red) per animal on day −3. On day 0, the animals were dosed subcutaneously with anti-CD37-(XII) conjugate, final (doses: 0.5 or 1.0 mg/kg).

In parallel, as part of study A, 30 female CB17-SCID mice bearing Raji-Luc tumor cells received a single intravenous (i.v.) treatment with anti-CD37-(XII) conjugate (doses: 0.5 or 1.0 mg/kg) or PBS. These data served as reference.

Tumor cell-dependent luciferase activity was determined weekly starting on day 1 after treatment by non-invasive whole-body bio-imaging (Caliper IVIS). Body weight was determined in parallel. Clinical signs and survival were monitored daily. Animals were sacrificed and necropsy was performed when one or more of the termination criteria applied or at study termination (day 97).

### Results and Conclusion

Intravenous inoculation of luciferase-expressing human Burkitt's lymphoma (Raji Luc) in female CB17-SCID (PBS Control Group 1) mice caused progressively increased luciferase-dependent whole body luminescence, body weight loss after day 11 and 100% mortality within 18 days after treatment. Mortality was mainly associated with marked hind limb paralysis.

Anti-CD37-(XII) conjugate controlled the Raji-Luc tumor-dependent mean bioluminescence close to the level on day 1 in 10 of 10 (100%, 0.5 mg/kg i.v. and 0.5 or 1.0 mg/kg s.c.) or in 8 of 10 (80%, 1.0 mg/kg i.v.) animals. The body weight increased mostly continuously until study end. Most frequent clinical findings and necropsy results may represent effects derived by Raji_Luc tumor and are not considered test item-related.

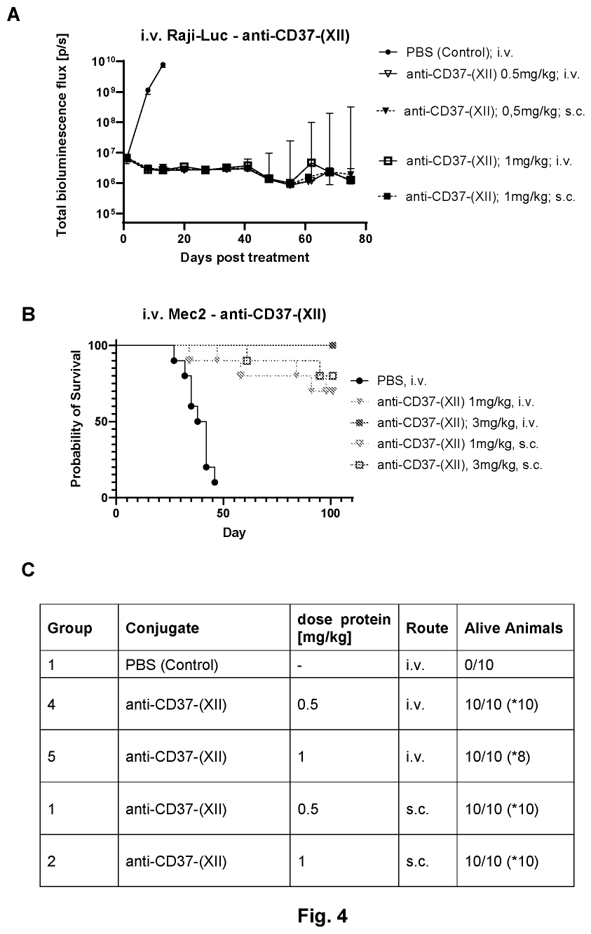

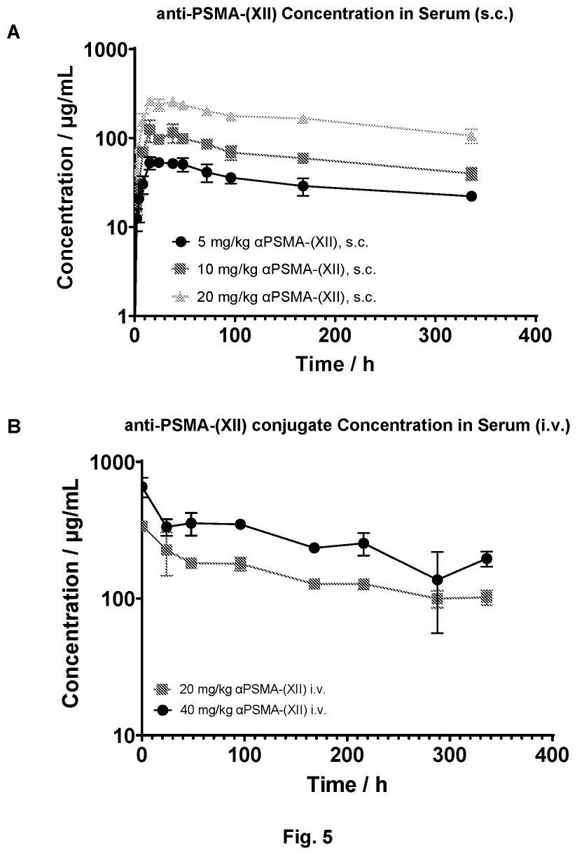

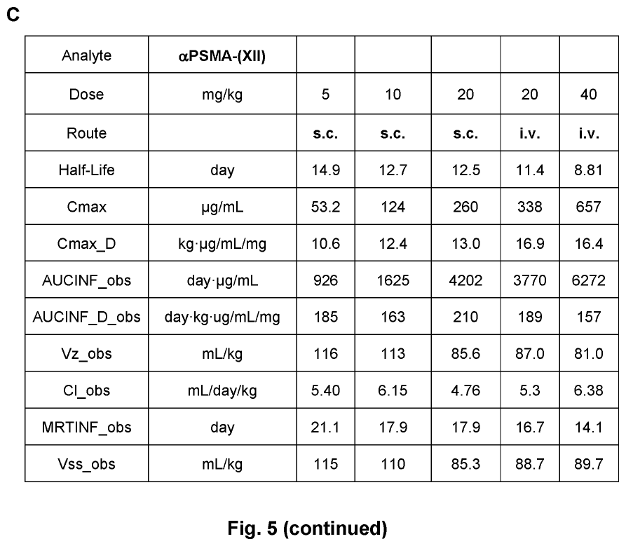

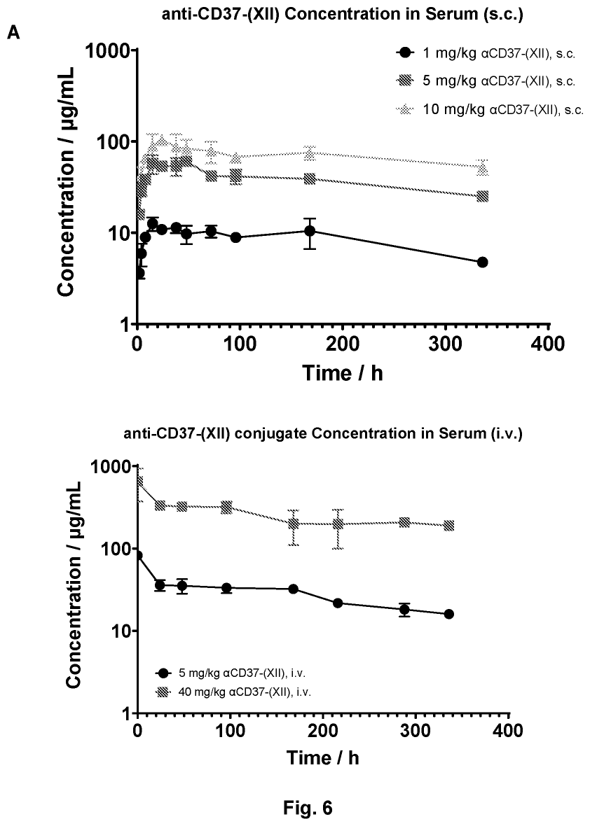

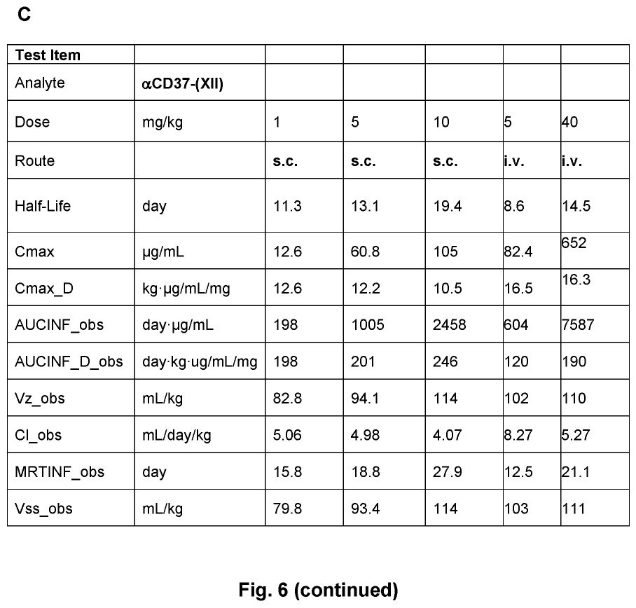

In conclusion, a single subcutaneous dose of 0.5 or 1.0 mg/kg anti-CD37-(XII) on day 0 (tumor inoculation on day −3) resulted in a very strong inhibitory effect (in 10 of 10 animals) on intravenous human Burkitt's lymphoma growth relative to PBS (i.v.), a normal body weight gain and a survival rate of 100% on study end (day 97). There are no statistically significant differences in bioluminescence between the animals treated with the same dose (0.5 or 1.0 mg/kg) but different (s.c. or i.v.) administration route. The results are shown in FIGS. 4A and 4C.

### Example 5: Efficacy of the Anti-CD37-(XII) Conjugate in a Disseminated MEC-2 Tumor Xenograft Model in Female CB-17 SCID Mice

The study consisted of 2 experimental groups with 10 animals each. 20 animals were inoculated intravenously with 2.5×10⁶ MEC-2 (human CLL) tumor cells in 200 μL PBS on day −3. Therapy was be initiated on day 0 (three days post tumor cell inoculation). Mice were treated with anti-CD37-(XII) as indicated in FIG. 4B. Body weight was determinated twice per week. Clinical signs and survival were recorded daily. Animals were sacrificed, and a necropsy performed, when termination criteria arise, or for ethical reasons. The results of the study are shown in FIG. 4B indicating that there is no statistically significant differences between the animals treated with the same dose but different (s.c. or i.v.) administration route.

### Example 6: Quantification of αPSMA-(XII) Conjugate Concentration in Serum

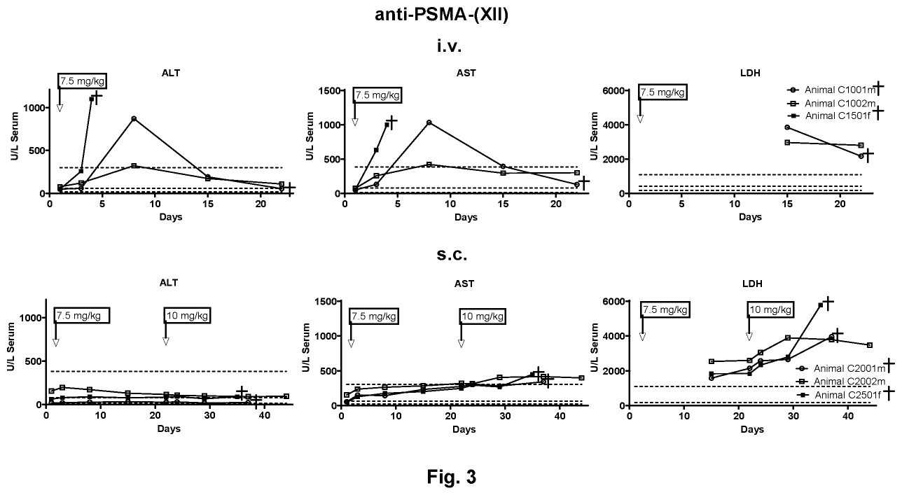

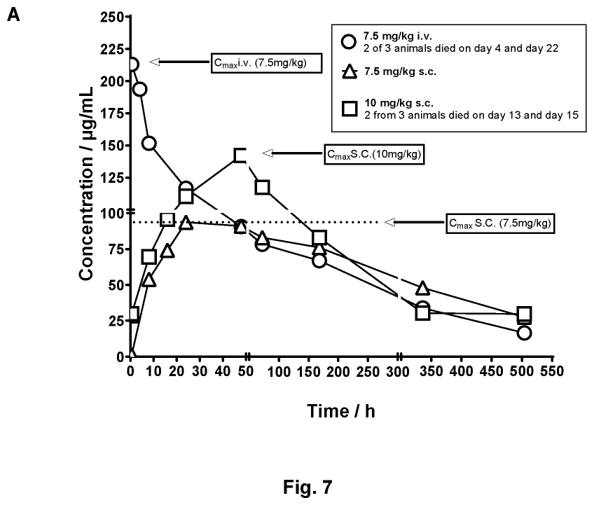

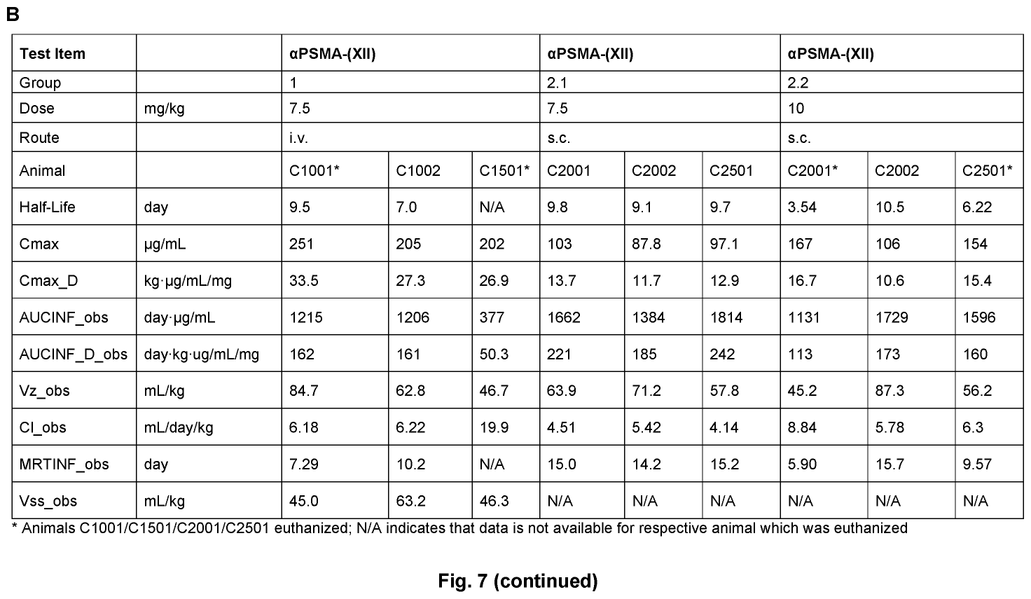

A sandwich ELISA assay was used to quantify serum concentrations of αPSMA-(XII) conjugate originating from the pharmacokinetic study of αPSMA-(XII) conjugate following single intravenous infusion or subcutaneous administration of the αPSMA-(XII) conjugate in cynomolgus monkey. For sandwich ELISA, a polyclonal anti-Amanitin antibody was used to capture the conjugate from cynomolgus monkey serum samples. A rabbit anti-human IgG H&L HRP antibody was used for detection. The study samples were analyzed in duplicates and the calibration range of the assay was from 1.6 to 200 pM. The optical density (OD) was measured at wavelengths of 450 and 570 nm and the difference-OD was further used for calculation. Serum concentrations of αPSMA-(XII) conjugate were calculated from calibration curve and retransformed into linear range. The results are shown in FIG. 7A, B. Administration of the αPSMA-(XII) conjugate as disclosed herein intravenously at a concentration of 7.5 mg/kg resulted in a Cmax of about 219 μg/ml (average of 3 animals), while the subcutaneous administration resulted in a Cmax of about 96 μg/ml at a dose of 7.5 mg/kg of the conjugate and about 142 μg/ml at a dose of 10 mg/kg the conjugate αPSMA-(XII). Tolerability of the αPSMA-(XII) conjugate was also increased as none of the animals dies at a dose of 7.5 mg/kg when administered subcutaneously compared to the i.v. administration route which resulted in the death of 2 out of 3 animals (see FIG. 7 as indicated). The increased tolerability of conjugate αPSMA-(XII) is also reflected in by the concentration of liver enzymes ALT and AST as shown in FIG. 3. Here, the i.v. administration of conjugate αPSMA-(XII) conjugate resulted in an increase of both markers which peaked between 5 to 10 days after i.v. administration of the conjugate, while the subcutaneous administration of the conjugate at the same dose only resulted in a minor increase of both liver enzymes. Thus, subcuteanous administration of conjugate αPSMA-(XII) results in a reduced Cₘₐₓ. A reduced Cₘₐₓ in turn results in an increased tolerability as shown in FIG. 3.

### Example 6: Subcutaneus Dosing (s.c.) Using Anti-GCC (Guanylyl Cyclase C (GUCY2C)) Conjugates

### MTD With Anti-GCC ATACs

The Maximum Tolerated Dose (MTD) of αGCC antibody conjugates comprising amatoxin-linker moieties (XIV), (XII) was determined after intravenous or subcutaneous administration of DAR2 ADCs (αGCC-LALA-D265C-(XIV) and αGCC-LALA-D265C-(XII)) in female NOD/SCID mice, whereby the respective anti-GCC antibody comprises heavy and light chain sequences according to SEQ ID NO: 3 and SEQ ID NO: 4. The conjugates were administered on day 0, body weight was determined twice per week and clinical observations and mortalities were recorded daily. The initial dose was increased or reduced in subsequent groups to define a Maximum Tolerated Dose (MTD), as shown in Table 1 below.

TABLE 1

| Conjugate | Route | MTD [mg/kg] |
|---|---|---|
| αGCC-LALA-D265C -(XIV) | i.v. | 4 |
| | s.c. | <10 |
| αGCC-LALA-D265C-(XII) | i.v. | 15 |
| | s.c. | 30 |

### Anti-Tumor Efficacy of the Amanitin-Based Anti-GCC ADCs

Anti-tumor efficacy of the anti-GCC ADCs anti-GCC-LALA-D265C-(XII) and anti-GCC-LALA-D265C-(XIV) was assessed after single subcutaneous dosing in a HEK293-GUCY2C-(HDP)-2B3 subcutaneous xenograft model in female NOD/SCID. Female NOD/SCID mice were inoculated subcutaneously with 5.0×10⁶ HEK293-GUCY2C-(HDP)-2B3 tumor cells (in 200 μL RPMI medium w/o PR containing 50% GFR Matrigel, Corning 356231) per animal into their right flanks. Once a mean tumor volume of 150-160 mm³ was reached 50 of these animals were selected and allocated to 5 groups with 10 animals each according Efficacy anti-GCC ATACs to tumor size. On the same or next day (day 1), the animals received a single s.c. or i.v. dose of anti-GCC-LALA-D265C-(XII) ADC (2.5 mg/kg) or anti-GCC-LALA-D265C-(XIV) ADC (6.0 mg/kg), respectively or PBS as control. Tumor volume was measured three times a week by caliper and body weight was determined in parallel. Clinical signs and survival were monitored daily. Animals were sacrificed and necropsy was performed when one or more of the termination criteria applied or at study termination at day 61.

### Results

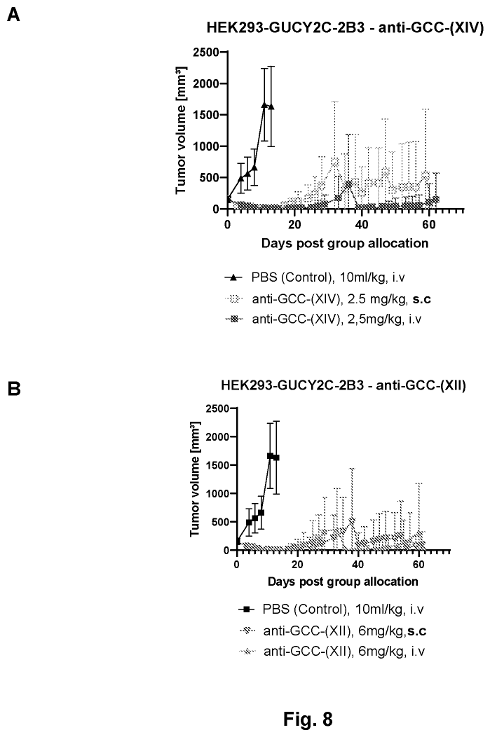

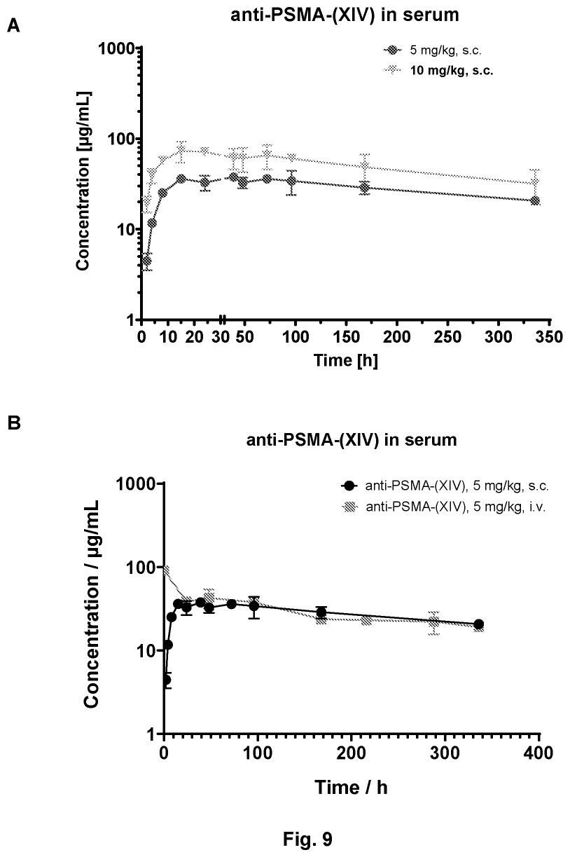

A single s.c. or i.v. dose of 2.5 mg/kg anti-GCC-LALA-D265C-(XIV) ADC or 6.0 mg/kg anti-GCC-LALA-D265C-(XII) ADC reduced the mean tumor volume (statistically significantly relative to PBS Control) within about 12 days after treatment, irrespective of the toxin-linker or administration route and led to (nearly) permanently complete (0 mm³) tumor remission in 3 and 7 (anti-GCC-LALA-D265C-(XIV), s.c. and i.v., respectively) or 5 and 8 (anti-GCC-LALA-D265C-(XII) ADC, s.c. and i.v., respectively) out of 10 animals. The results of are depicted in FIG. 8.

In conclusion, a single subcutaneous dose of 2.5 mg/kg anti-GCC-LALA-D265C-(XIV) or 6.0 mg/kg anti-GCC-LALA-D265C-(XII) ADC resulted in a strong inhibitory effect on subcutaneous human embryonic kidney carcinoma growth, a body weight gain slightly delayed relative to PBS Control and a survival rate of about 30 and 60% (day 61), respectively. There are no statistically significant differences in tumor inhibition, body weight development and survival between the animals treated with the same test item and dose but different (s.c. or i.v.) administration route.

HEK293-GUCY2C-HDP-2B3 cells which were used in the above xenograft model express human GUCY2C and have been described in PCT/EP2023/080350. In brief, HEK293-GUCY2C-HDP-2B3 were obtained by transient transfection of HEK293 wild-type cells with an overexpression plasmid encoding human guanylate cyclase 2C (GUCY2C or GCC) having the amino acid sequence according to Uniprot no. P25092, (version 13 Sep. 2023) and a resistance against geneticin (G418). Selection of stably transfected cells was done after 4 days by placing the cells in culture medium containing G418 to select for a stable cell pool expressing GCC and G418 resistance. A stable single-cell clone was isolated from this cell pool by using limiting dilution. GCC surface expression was confirmed for the stable cell clone HEK293-GUCY2C-HDP-2B3 by flow cytometry and cytotoxicity assay (BrdU ELISA), respectively.

### Example 7 Pharmacokinetics of s.c.-Administered Anti-PSMA-(XIV) ADC

Serum pharmacokinetics of amanitin-based anti-PSMA ADC h3/F11-LALA-D265C Var16-(XIV) was assessed, following a single s.c. administration of a 10 or 5 mg/kg dose or i.v. administration of a 5 mg/kg dose to male CB17-SCID mice. Male CB17-SCID mice were allocated to cohorts (according to sample collection time points, 3 animals each) in groups with 18 animals each. Animals received a single s.c. (5 or 10 mg/kg) or i.v. (5 mg/kg) dose of h3/F11-LALA-D265C Var16-30.2347. Body weight was determined up to 14 days twice a week. Clinical symptoms and mortality were recorded daily. Blood samples were collected at 12 time points from 5 min up to 336 hours (14 days) after administration. Pharmacokinetic parameters of h3/F11-LALA-D265C Var16-(XIV) in serum were calculated and compared.

### Results

Subcutaneous dosing with 5.0 mg/kg h3/F11-LALA-D265C Var16-(XIV) revealed a preferable PK profile with a slightly increased dose normalized AUC and half-life and a strongly decreased dose normalized Cₘₐₓ in comparison to intravenous dosing with the same dose.

After s.c. dosing, Cₘₐₓ of the 5 and 10 mg/kg dose of h3/F11-LALA-D265C Var16-(XIV) were dose proportional with C_{max_D} values of 7.54 and 7.33 kg·μg/mL/mg, respectively. The half-life (13.8 and 11.0 days), dose normalized AUC (161 and 119 day·kg·μg/mL/mg) and clearance (6.2 and 8.4 mL/day/kg) was in a similar range.

Taking into account the results of the pharmacokinetics for the s.c.-administered anti-PSMA-(XII) ADC (FIG. 7), these results support the finding that the effect of s.c. administration on the pharmacokinetics of an amanitin-based ADC is independent of the amanitin derivative used as payload.

### REFERENCES

Almagro and Fransson 2008, Front Biosci. 2008 Jan. 1;13:1619-33.).
Armour, et al., Eur. J. Immunol. 29(8) (1999) 2613-2624
Binz et al., 2005, Curr Opin Biotechnol. 2005 August;16(4):459-69
Carl et al., J. Med. Chem. (1981) 24:479-480;
Carter, Biochem. J. (1986) Vol. 237: 1-7,
Chakravarty et al (1983) J. Med. Chem. 26:638-644;
de Groot et al (2001) J. Org. Chem. 66:8815-8830;
Dornan et al (2009) Blood 114(13):2721-2729; U.S. Pat. No. 7,521,541
Dubowchik and Walker, 1999, Pharm. Therapeutics 83:67-123
Edelman, G. M. et al., Proc. Natl. Acad. USA, 63, 78-85 (1969).
Fang et al. Anal Biochem. 2015 Jul. 1;480:74-81.
Hay et al. (1999) Bioorg. Med. Chem. Lett. 9:2237
Hollinger et al. Proc Natl Acad Sci USA. 1993 Jul. 15;90(14):6444-8
de Groot et al (2001) J. Org. Chem. 66:8815-8830;
International Union of Pure and Applied Chemistry, ISBN: 0-9678550-9-8.
Jain et al. (2015) Pharm Res 32:3526-40.
Junutula J R et al. (2008). Nat Biotechnology Vol. 26: 925-932.
Leriche et al., Bioorg. Med. Chem., 20:571-582, 2012
Liu et al Nature. 2015 Apr. 30; 520(7549): 697-701.
Merz et al. Am J Hematol. 2016 November;91(11):E473-E477
Neville et al., 1989, Biol. Chem. 264:14653-14661
Rathanaswami et al. Analytical Biochemistry, Vol. 373:52-60, 2008;
Riechmann et al., 1988, Nature 332:323-327,
Shields, et al., J. Biol. Chem. 276(9) (2001) 6591-6604
Thorpe et al., 1987, Cancer Res. 47:5924-5931
Villalba M I et al. J Biol Chem. (2015) Vol. 290(5): 2577-2592.
Voss et al. BMC Molecular Biology 2014, 15:7
Wieland T, Faulstich H., CRC Crit Rev Biochem. 5 (1978) 185-260),
Wilkinson et al. PLOS One. 2021; 16(12): e0260954
Yadav, et al. J. Biol. Chem. 2015, 290, 29732-29741

### PATENT LITERATURE REFERENCED

EP3043774A1
W098/13059
WO 01/14424
W02004/032828,
WO2005/084390 A2
WO2005/112919
WO2006/121168
WO2007011968A2
WO2009/101611
WO2009/114335
WO2009/052249
WO2016/142049
WO2016027859A1
WO2018/220169.
WO2018/115466:
WO19192979 A1
WO2012135345A1
WO2016040856A2
WO 2021/234402 A2
WO2022/096604 A1,
US20030130189;
US20030096743;
US20040052793;
US20040018194
US20040052793
US20040121940;
U.S. Pat. No. 8,008,449
U.S. Pat. No. 8,354,509 and
U.S. Pat. No. 6,835,807
U.S. Pat. No. 6,268,488
U.S. Pat. No. 5,621,002
U.S. Pat. No. 6,677,435;
U.S. Pat. No. 6,218,519
U.S. Pat. No. 6,759,509;
U.S. Pat. No. 5,530,101;
U.S. Pat. No. 5,585,089;
U.S. Pat. No. 5,693,761;
U.S. Pat. No. 5,693,762;
U.S. Pat. No. 6,180,370.
U.S. Pat. No. 7,521,541;
U.S. Pat. No. 7,723,485
U.S. Pat. No. 5,122,368;
U.S. Pat. No. 5,824,805;
U.S. Pat. No. 5,622,929
U.S. Pat. No. 4,880,935
U.S. Pat. No. 5,122,368;
U.S. Pat. No. 5,824,805;
U.S. Pat. No. 5,622,929;
U.S. Pat. No. 9,605,051 B2
U.S. Pat. No. 11,377,497

```
<?xml version=“1.0” encoding=“UTF-8”?>
<!DOCTYPE ST26SequenceListing PUBLIC “-//WIPO//DTD Sequence Listing 1.3//EN”
“ST26SequenceListing_V1_3.dtd”>
<ST26SequenceListing
dtdVersion=“V1_3” fileName=“006033US-seqlist-as-filed.xml”
softwareName=“WIPO Sequence” softwareVersion=“2.3.0” productionDate=“2024-03-12”>
  <ApplicationIdentification>
    <IPOfficeCode>US</IPOfficeCode>
    <ApplicationNumberText></ApplicationNumberText>
    <FilingDate></FilingDate>
  </ApplicationIdentification>
  <ApplicantFileReference>006033US</ApplicantFileReference>
  <EarliestPriorityApplicationIdentification>
    <IPOfficeCode>EP</IPOfficeCode>
    <ApplicationNumberText>23161638.4</ApplicationNumberText>
    <FilingDate>2023-03-13</FilingDate>
  </EarliestPriorityApplicationIdentification>
  <ApplicantName languageCode=“en”>Heidelberg Pharma Research
GmbH</ApplicantName>
  <InventionTitle languageCode=“en”>Subcutaneously administered antibody-drug
conjugates for use in cancer treatment</InventionTitle>
  <SequenceTotalQuantity>6</SequenceTotalQuantity>
  <SequenceData sequenceIDNumber=“1”>
    <INSDSeq>
      <INSDSeq_length>449</INSDSeq_length>
      <INSDSeq_moltype>AA</INSDSeq_moltype>
      <INSDSeq_division>PAT</INSDSeq_division>
      <INSDSeq_feature-table>
        <INSDFeature>
          <INSDFeature_key>source</INSDFeature_key>
<INSDFeature_location>1..449</INSDFeature_location>
          <INSDFeature_quals>
            <INSDQualifier>
<INSDQualifier_name>mol_type</INSDQualifier_name>
<INSDQualifier_value>protein</INSDQualifier_value>
            </INSDQualifier>
            <INSDQualifier id=“q2”>
<INSDQualifier_name>organism</INSDQualifier_name>
<INSDQualifier_value>synthetic construct</INSDQualifier_value>
            </INSDQualifier>
          </INSDFeature_quals>
        </INSDFeature>
      </INSDSeq_feature-table>
<INSDSeq_sequence>QVQLVQSGAEVKKPGASVKVSCKASGYIFISSAMHWVRQAPGQRLEWMGLINPGNGNTKYSQKFQ
GRVTITRDTSASTAYMELSSLRSEDTAVYYCARAYFRGLGFDIWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVCVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK</INSDSeq_sequence>
    </INSDSeq>
  </SequenceData>
  <SequenceData sequenceIDNumber=“2”>
    <INSDSeq>
      <INSDSeq_length>214</INSDSeq_length>
      <INSDSeq_moltype>AA</INSDSeq_moltype>
      <INSDSeq_division>PAT</INSDSeq_division>
      <INSDSeq_feature-table>
        <INSDFeature>
          <INSDFeature_key>source</INSDFeature_key>
<INSDFeature_location>1..214</INSDFeature_location>
          <INSDFeature_quals>
            <INSDQualifier>
<INSDQualifier_name>mol_type</INSDQualifier_name>
<INSDQualifier_value>protein</INSDQualifier_value>
            </INSDQualifier>
            <INSDQualifier id=“q4”>
<INSDQualifier_name>organism</INSDQualifier_name>
<INSDQualifier_value>synthetic construct</INSDQualifier_value>
            </INSDQualifier>
          </INSDFeature_quals>
        </INSDFeature>
      </INSDSeq_feature-table>
<INSDSeq_sequence>DIQMTQSPSSLSASAGDRVTITCRASQSISSYLNWYQQKPGKAPKLLIYAASSLQSGVPSRFSGS
GSGTDFTLAISSLQPEDFATYYCQQSYSTPWTFGQGTKVDIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC</INSDSeq_sequenc
e>
    </INSDSeq>
  </SequenceData>
  <SequenceData sequenceIDNumber=“3”>
    <INSDSeq>
      <INSDSeq_length>214</INSDSeq_length>
      <INSDSeq_moltype>AA</INSDSeq_moltype>
      <INSDSeq_division>PAT</INSDSeq_division>
      <INSDSeq_feature-table>
        <INSDFeature>
          <INSDFeature_key>source</INSDFeature_key>
<INSDFeature_location>1..214</INSDFeature_location>
          <INSDFeature_quals>
            <INSDQualifier>
<INSDQualifier_name>mol_type</INSDQualifier_name>
<INSDQualifier_value>protein</INSDQualifier_value>
            </INSDQualifier>
            <INSDQualifier_id=“q6”>
<INSDQualifier_name>organism</INSDQualifier_name>
<INSDQualifier_value>synthetic construct</INSDQualifier_value>
            </INSDQualifier>
          </INSDFeature_quals>
        </INSDFeature>
      </INSDSeq_feature-table>
<INSDSeq_sequence>DIRMTQSPSSLSASAGDRVTITCRASQSISSYLNWYQQKPGKAPKLLIYAASSLQSGVPSRFSGS
GSGTDFTLTISSLQPEDFATYYCQQSYSTPLTFGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC</INSDSeq_sequenc
e>
    </INSDSeq>
  </SequenceData>
  <SequenceData sequenceIDNumber=“4”>
    <INSDSeq>
      <INSDSeq_length>449</INSDSeq_length>
      <INSDSeq_moltype>AA</INSDSeq_moltype>
      <INSDSeq_division>PAT</INSDSeq_division>
      <INSDSeq_feature-table>
        <INSDFeature>
          <INSDFeature_key>source</INSDFeature_key>
<INSDFeature_location>1..449</INSDFeature_location>
          <INSDFeature_quals>
            <INSDQualifier>
<INSDQualifier_name>mol_type</INSDQualifier_name>
<INSDQualifier_value>protein</INSDQualifier_value>
            </INSDQualifier>
            <INSDQualifier_id=“q8”>
<INSDQualifier_name>organism</INSDQualifier_name>
<INSDQualifier_value>synthetic construct</INSDQualifier_value>
            </INSDQualifier>
          </INSDFeature_quals>
        </INSDFeature>
      </INSDSeq_feature-table>
<INSDSeq_sequence>QVQLVQSGAEVKKPGASVKVSCKASGYIFISSAMHWVRQAPGQRLEWMGLINPGNGNTKYSQKFQ
GRVTITRDTSASTAYMELSSLRSEDTAVYYCARAYFRGLGFDIWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTHTCPPC
PAPEAAGGPSVFLFPPKPKDTLMISRTPEVTCVVVCVSHEDPEVKFNWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSPGK</INSDSeq_sequence>
    </INSDSeq>
  </SequenceData>
  <SequenceData sequenceIDNumber=“5”>
    <INSDSeq>
      <INSDSeq_length>216</INSDSeq_length>
      <INSDSeq_moltype>AA</INSDSeq_moltype>
      <INSDSeq_division>PAT</INSDSeq_division>
      <INSDSeq_feature-table>
        <INSDFeature>
          <INSDFeature_key>source</INSDFeature_key>
<INSDFeature_location>1..216</INSDFeature_location>
          <INSDFeature_quals>
            <INSDQualifier>
<INSDQualifier_name>mol_type</INSDQualifier_name>
<INSDQualifier_value>protein</INSDQualifier_value>
            </INSDQualifier>
            <INSDQualifier_id=“q10”>
<INSDQualifier_name>organism</INSDQualifier_name>
<INSDQualifier_value>synthetic construct</INSDQualifier_value>
            </INSDQualifier>
          </INSDFeature_quals>
        </INSDFeature>
      </INSDSeq_feature-table>
<INSDSeq_sequence>QSALTQPASVSGSPGQSITISCTGTSSDVGSYNLVSWYQQHPGKAPKLMIYEGSKRPSGVSNRFS
ASKSGNTASLTISGLQTEDEADYYCSSYVPRSSLVFGGGTKLTVLGQPKAAPSVTLFPPSSEELQANKATLVCLISDFYPGAV
TVAWKADSSPVKAGVETTTPSKQSNNKYAASSYLSLTPEQWKSHRSYSCQVTHEGSTVEKTVAPTECS</INSDSeq_seque
nce>
    </INSDSeq>
  </SequenceData>
  <SequenceData sequenceIDNumber=“6”>
    <INSDSeq>
      <INSDSeq_length>452</INSDSeq_length>
      <INSDSeq_moltype>AA</INSDSeq_moltype>
      <INSDSeq_division>PAT</INSDSeq_division>
      <INSDSeq_feature-table>
        <INSDFeature>
          <INSDFeature_key>source</INSDFeature_key>
<INSDFeature_location>1..452</INSDFeature_location>
          <INSDFeature_quals>
            <INSDQualifier>
<INSDQualifier_name>mol_type</INSDQualifier_name>
<INSDQualifier_value>protein</INSDQualifier_value>
            </INSDQualifier>
            <INSDQualifier_id=“q12”>
<INSDQualifier_name>organism</INSDQualifier_name>
<INSDQualifier_value>synthetic construct</INSDQualifier_value>
            </INSDQualifier>
          </INSDFeature_quals>
        </INSDFeature>
      </INSDSeq_feature-table>
<INSDSeq_sequence>QVQLVQSGAEVKKPGSSVKVSCKASGGTFSTYTINWVRQAPGQGLEWMGRIIPVLGIANYAQKFQ
GRVTITADKSTSTAYMELSSLRSEDTAVYYCARDTPRLRSSYYMDVWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPKSCDKTHTC
PPCPAPEAAGGPSVFLFPPKPKDTLMISRTPEVTCVVVCVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK</INSDSeq_sequence>
    </INSDSeq>
  </SequenceData>
</ST26SequenceListing>
```

What is claimed is:

1. A pharmaceutical composition for use in the treatment of cancer, wherein the pharmaceutical composition comprises a conjugate, wherein the conjugate comprises (i) a target-binding moiety, (ii) at least one amatoxin, and (iii) at least one linker connecting said target binding moiety with said at least one amatoxin, wherein the pharmaceutical composition is administered subcutaneously, wherein the target-binding moiety of said conjugate is selected from the group of
(i) an antibody, preferably a monoclonal antibody,
(ii) an antigen-binding fragment thereof, preferably a variable domain (Fv), a Fab fragment or an F(ab)2 fragment,
(iii) an antigen-binding derivative thereof, preferably a single-chain Fv (scFv), and
(iv) an antibody-like protein.

2. The pharmaceutical composition for use according to claim 1, wherein the antibody of said conjugate is a murine, a chimeric, a humanized or a human antibody, preferably a humanized or human antibody.

3. The pharmaceutical composition for use according to claim 1, wherein the antibody of said conjugate is an IgG isotype antibody selected from an IgG1 isotype, an IgG2 isotype, an IgG3 isotype, or an IgG4 isotype antibody.

4. The pharmaceutical composition for use according to claim 3, wherein the antibody moiety of said conjugate does not induce antibody-dependent cell-mediated cytotoxicity (ADCC), antibody-dependent cellular phagocytosis((ADCP), or complement-dependent cytotoxicity (CDC).

5. The pharmaceutical composition for use according to claim 1, wherein the antibody moiety of said conjugate comprises an Fc region which comprises at least one amino acid substitution at position D265, L234, L235, or G236 (according to EU numbering system).

6. The pharmaceutical composition for use according to claim 1, wherein the Fc region of said antibody portion of said conjugate comprises at least one amino acid substitution selected from L234A, L234S, L234G, L235A, L235G, L235S, L235T, G236R and D265C (according to EU numbering system).

7. The conjugate for use according to claim 1, wherein the Fc region of said antibody comprises the amino acid substitution D265C (according to EU numbering system).

8. The pharmaceutical composition for use according to claim 1, wherein the Fc region of said antibody comprises the amino acid substitutions L234A, L235A and D265C (according to EU numbering system).

9. The pharmaceutical composition for use according to claim 1, wherein linker is connected to said antibody portion via any of the naturally occurring cysteine residues of said antibody, preferably via any of the naturally occurring cysteine residues which form the interchain disulfide bonds of said antibody and/or via a disulfide linkage.

10. The pharmaceutical composition for use according to claim 1, wherein the amatoxin of said conjugate is conjugated to said antibody via a cleavable or non-cleavable linker.

11. The pharmaceutical composition for use according to claim 10, wherein the cleavable linker of said conjugate is selected from the group consisting of an enzymatically cleavable linker, preferably a protease-cleavable linker, a chemically cleavable linker, preferably a linker comprising a disulfide bridge.

12. The pharmaceutical composition for use according to claim 11, wherein the enzymatically cleavable linker of said conjugate comprises a valine-alanine (Val-Ala), valine-citrulline (Val-Cit), valine-lysine (Val-Lys), valine-arginine (Val-Arg) dipeptide, a phenylalanine-lysine-glycine-proline-leucin-glycine (Phe Lys Gly Pro Leu Gly) or alanine-alanine-proline-valine (Ala Ala Pro Val) peptide, or a β-glucuronide or β-galactoside.

13. The pharmaceutical composition for use according to claim 12, wherein the enzymatically cleavable linker of said conjugate is a cathepsin B cleavable linker.

14. The pharmaceutical composition for use according to claim 13, wherein the cleavable linker is a self-immolative linker, wherein the self-immolative linker comprises a para- aminobenzyloxycarbonyl (PAB or PABC) moiety.

15. The pharmaceutical composition for use according to claim 1, wherein the linker of said conjugate is connected to said amatoxin via (i) the γ C-atom of amatoxin amino acid 1, or (ii) the δ C-atom of amatoxin amino acid 3, or (iii) the 6′-C-atom of amatoxin amino acid 4.

16. The pharmaceutical composition for use according to claim 1, wherein said conjugate comprises at least one amatoxin linker moiety selected from any of compounds (I)-(XI):

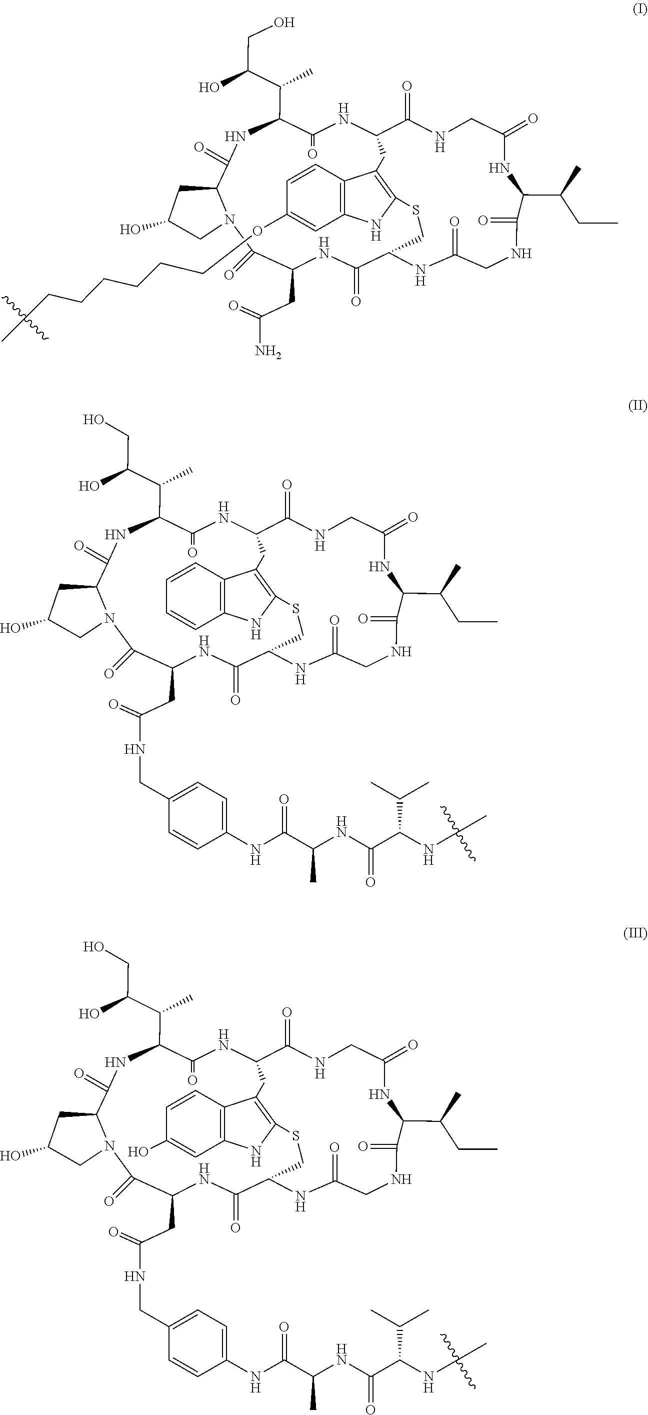

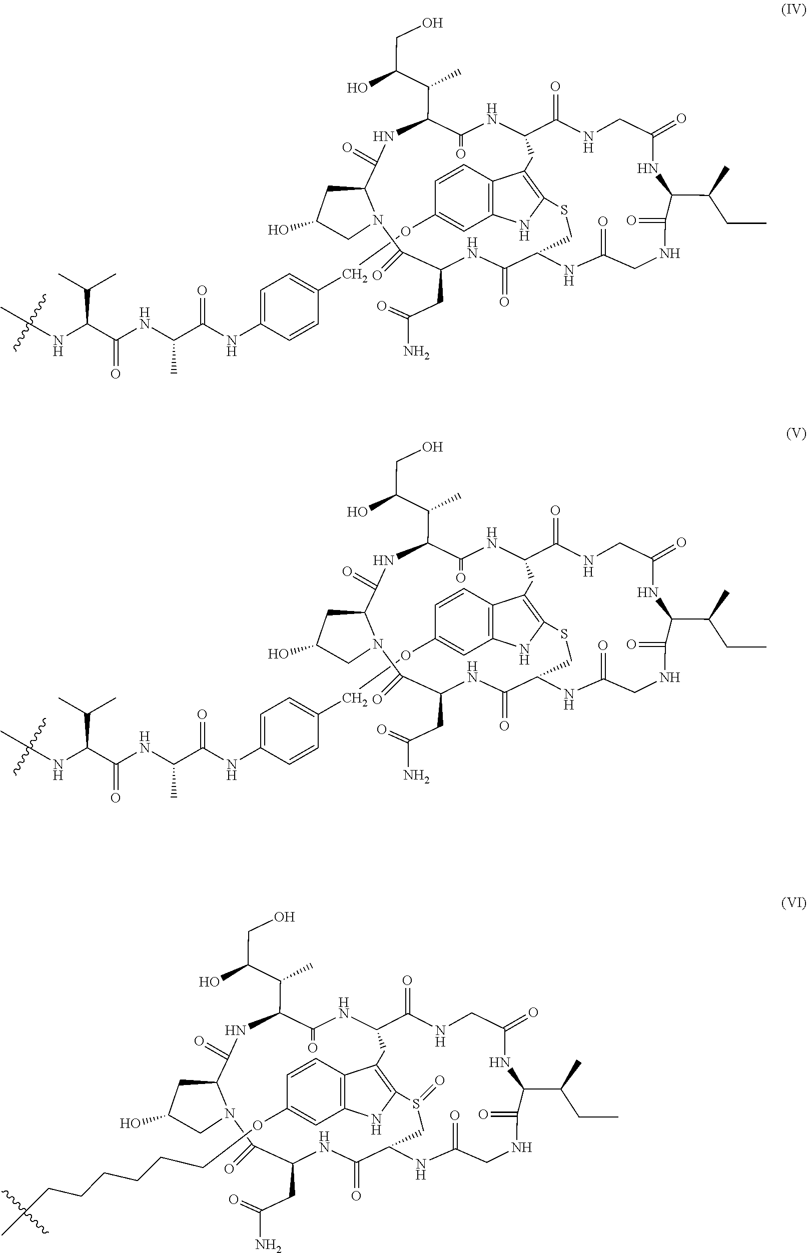

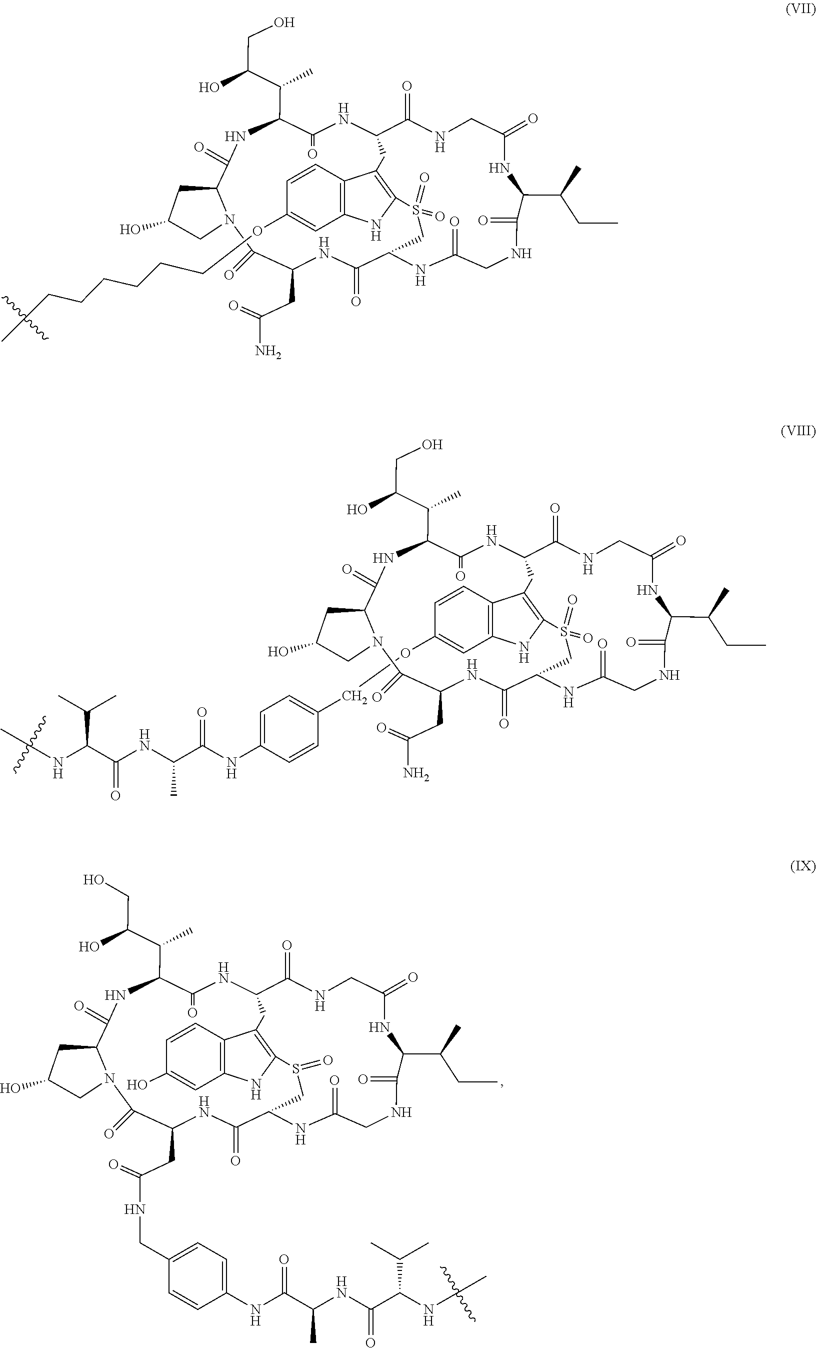

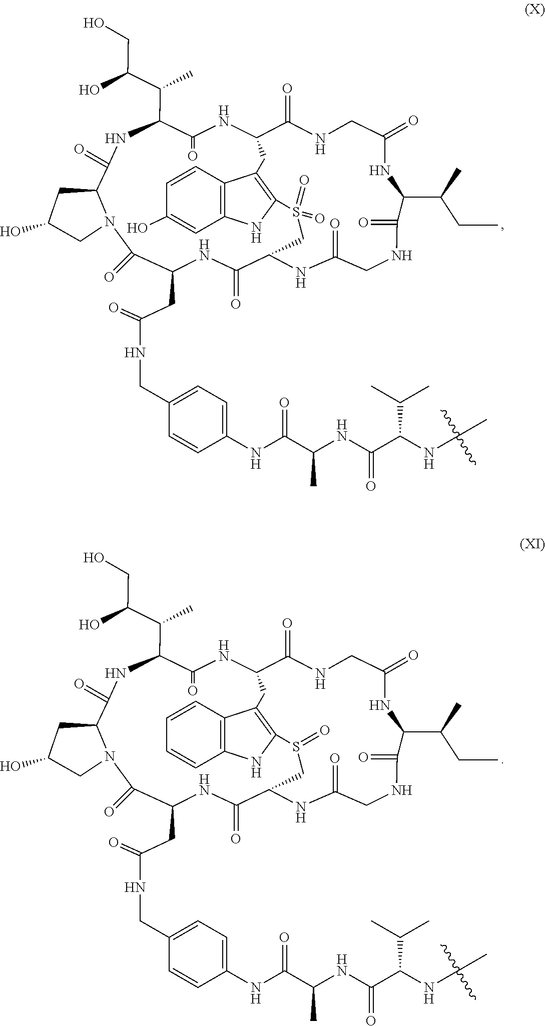 [SMILES: *N[C@H](C(=O)N[C@@H](C)C(=O)Nc1ccc(CNC(=O)C[C@@H]2NC(=O)[C@@H]3CS(=O)(=O)c4[nH]c5cc(O)ccc5c4C[C@H](NC(=O)[C@H]([C@@H](C)[C@@H](O)CO)NC(=O)[C@@H]4C[C@@H](O)CN4C2=O)C(=O)NCC(=O)N[C@@H]([C@@H](C)CC)C(=O)NCC(=O)N3)cc1)C(C)C ; *N[C@H](C(=O)N[C@@H](C)C(=O)Nc1ccc(CNC(=O)C[C@@H]2NC(=O)[C@@H]3CS(=O)c4[nH]c5ccccc5c4C[C@H](NC(=O)[C@H]([C@@H](C)[C@@H](O)CO)NC(=O)[C@@H]4C[C@@H](O)CN4C2=O)C(=O)NCC(=O)N[C@@H]([C@@H](C)CC)C(=O)NCC(=O)N3)cc1)C(C)C]

17. The pharmaceutical composition for use according to claim 1, wherein the antibody of said conjugate is conjugated to at least one amatoxin linker moiety via a thioether linkage according to any one of formula XII to XXII: 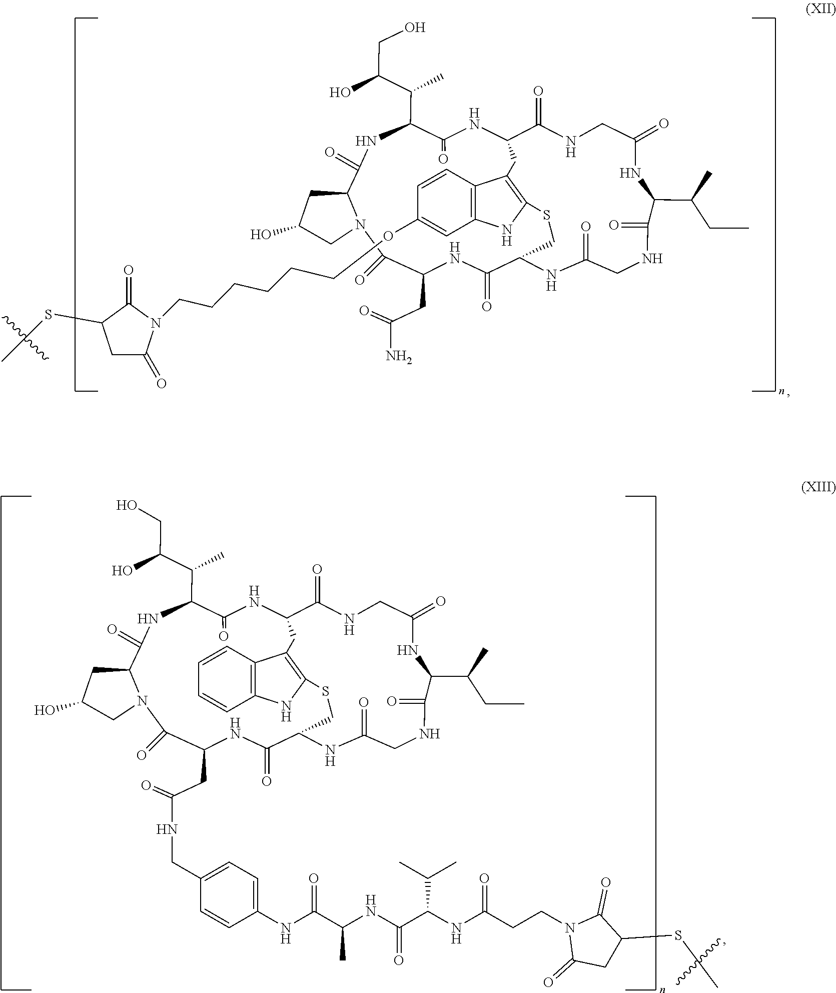 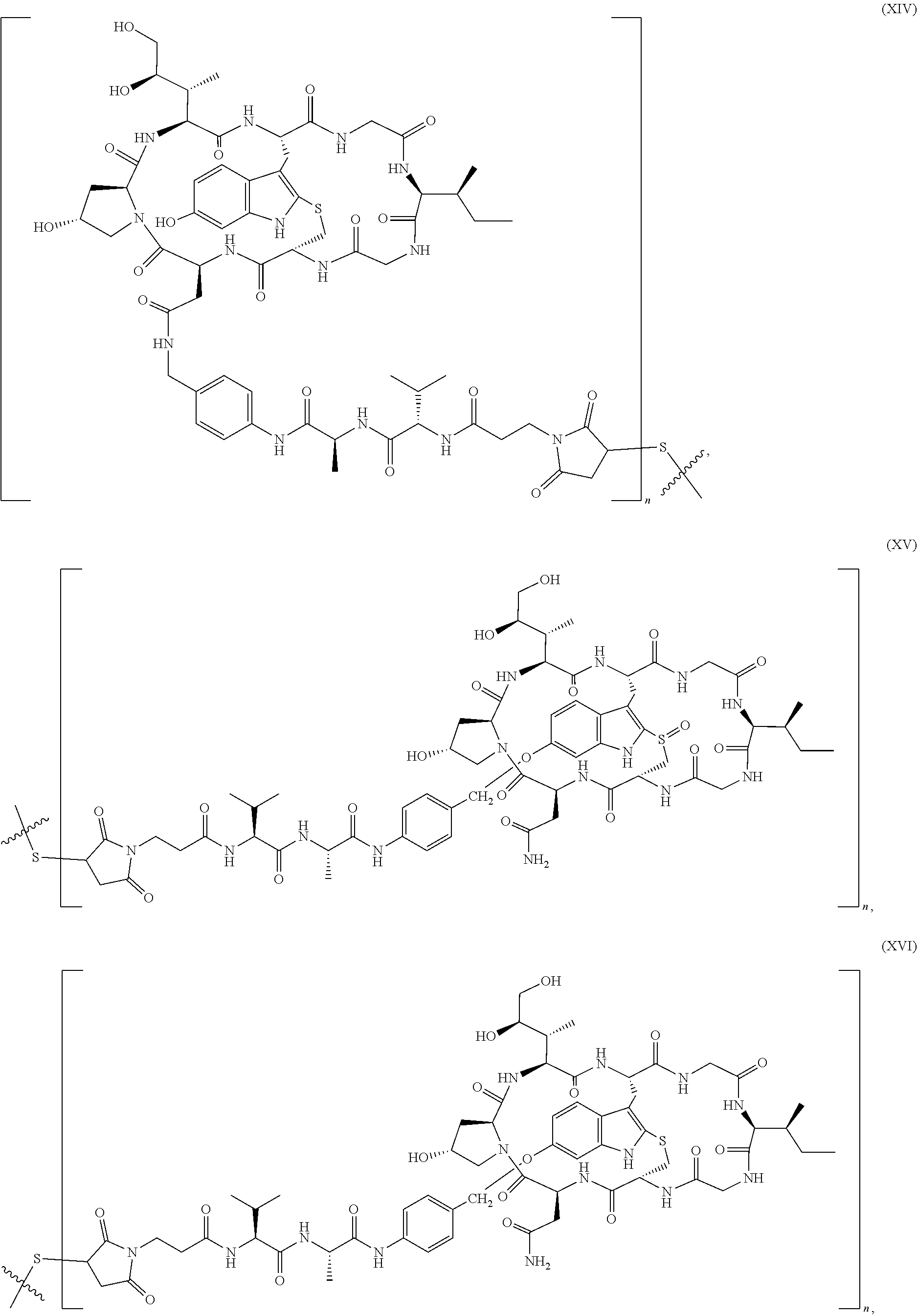 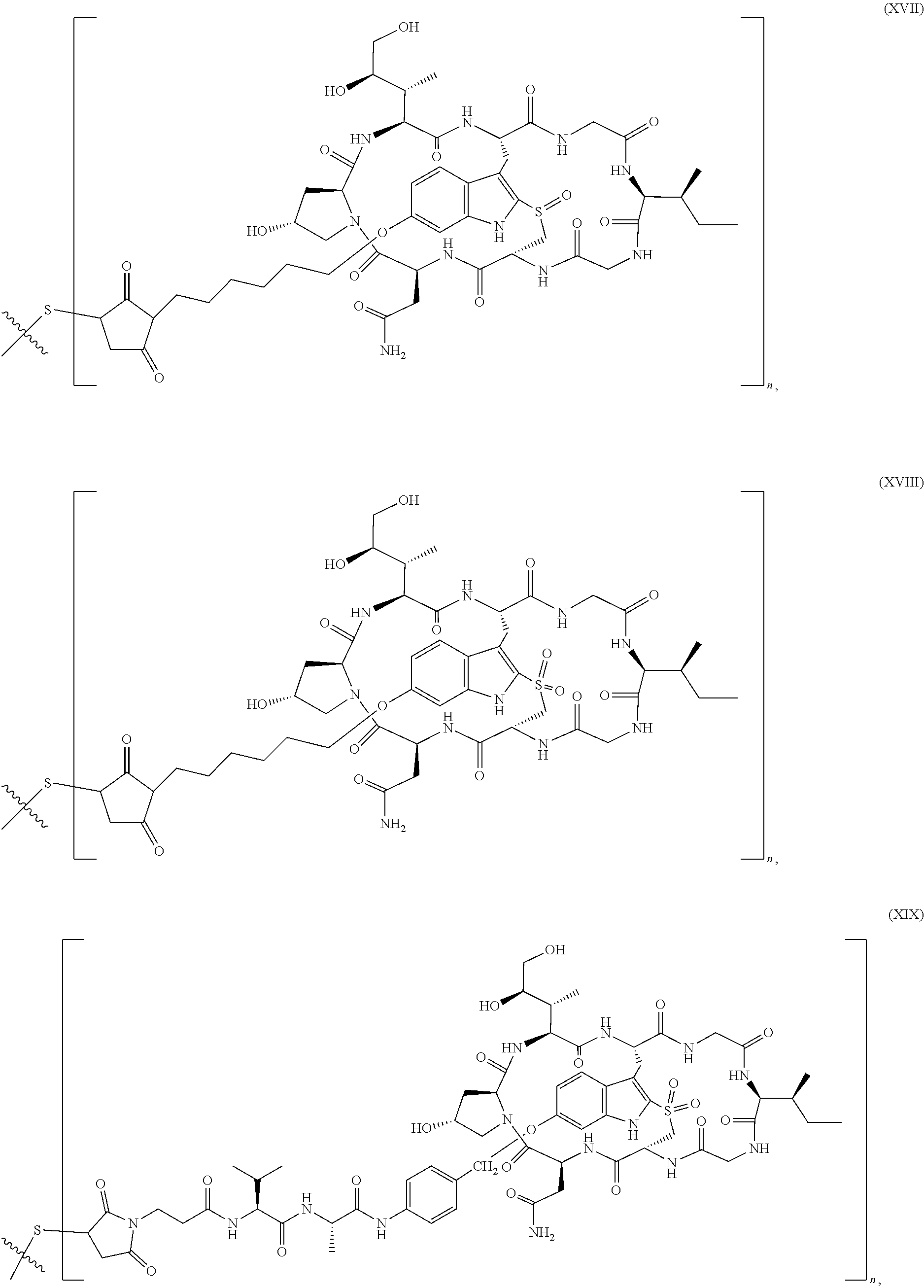 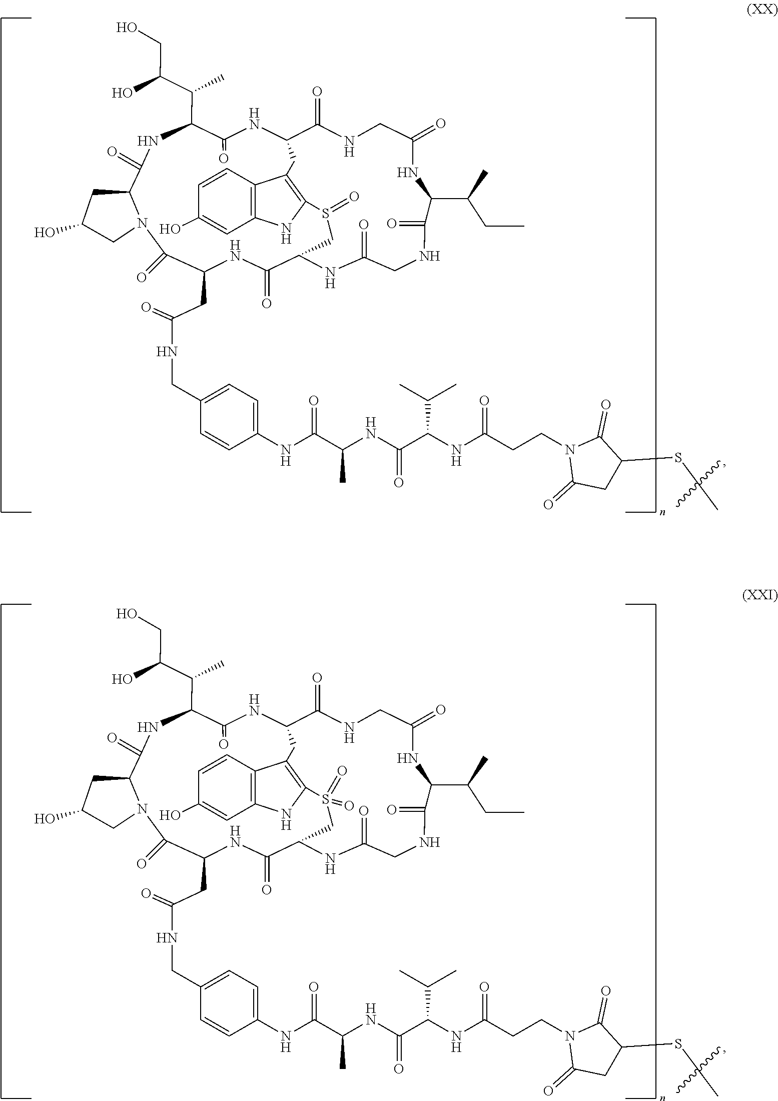 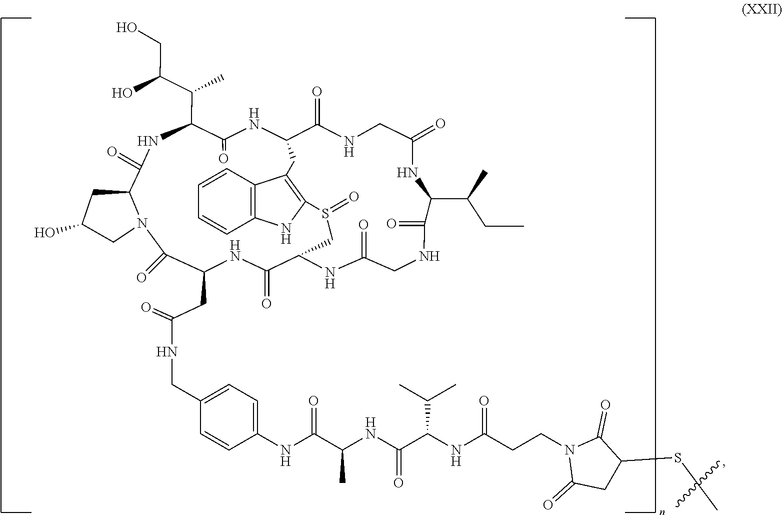
wherein said amatoxin-linker moieties are coupled to the thiol groups of cysteine residues of the antibody portion of said conjugate, wherein n is from about 1, 2, 3 to about 4, 5, 6, 7, or 8.

18. The pharmaceutical composition for use according to claim 19, wherein n is from about 1.5, or 2 to about 3 or 3.5, preferably wherein n is about 2.

19. The pharmaceutical composition for use according to claim 1, wherein the cancer is a solid tumor, or a non-solid tumor selected from the group comprising gastric carcinoma, adenocarcinoma, melanoma, ovarian carcinoma, uterine cancer, cervical cancer, breast cancer, including triple negative breast cancer, bronchial carcinoma, Ewing's sarcoma, liposarcoma, fibrosarcoma, leiomyosarcoma, thymoma, testicular cancer, neuroblastoma, glioma, prostate cancer, castration-resistant prostate cancer, gastrointestinal cancer, colorectal cancer, metastatic colorectal cancer (mCRC), stomach cancer, esophageal cancer, cancer of the larynx, cancer of the parotid, cancer of the biliary tract, rectal cancer, endometrial cancer, desmoid tumors, desmoplastic small round cell tumors, neuroectodermal tumors, retinoblastomas, rhabdomyosarcomas, Wilms tumors, osteosarcoma, Hodgkin-lymphoma, follicular lymphoma, diffuse large B cell non-Hodgkin's lymphoma (DBNHL), subtypes of non-Hodgkin's lymphoma including mantle cell lymphoma (MCL), chronic lymphocytic leukemia (CLL), Richter syndrome, primary cutaneous marginal zone lymphoma (PCMZL), hairy cell leukemia, acute myeloid leukemia (AML), or multiple myeloma.

20. The pharmaceutical composition for use according to claim 19, wherein the cancer cells or the tumor are characterized by a hemizygous loss of TP53, POLR2A, or del(17p13), or wherein at least 1%, 2.5%, 5%, 7.5%, 10%, 12.5%, 15%, 20%, 25%, 30%, 40%, or 50% or more of the tumor cells are characterized by said hemizygous loss of TP53, POLR2A, or del(17p13).

21. The pharmaceutical composition for use according to claim 1, further comprising one or more pharmaceutically acceptable buffers, surfactants, diluents, carriers, excipients, fillers, binders, lubricants, glidants, disintegrants, adsorbents, and/or preservatives.

22. The pharmaceutical composition for use according to claim 1, wherein the pharmaceutical composition is co-administered with an immune checkpoint inhibitor.

23. The pharmaceutical composition for use according to claim 22, wherein the pharmaceutical composition further comprises a recombinant hyaluronidase.

24. The pharmaceutical composition for use according to claim 1, wherein the antibody of said conjugate specifically binds to a cell surface antigen on a tumor cell, or to a tumor-associated antigen.

25. The pharmaceutical composition for use according to claim 24, wherein the cell surface antigen on said tumor cell is selected from the group EpCAM, HER2/neu, EGFR (HER1, ErbB1), TROP-2, BCMA, CD37, STEAP1, FXYD3, CA125, CD30, NCAM (CD56), MUC1, CEA (CD66e), VEGF, AFP, AXL, TYRO3, MER, CD20, CD19, CD52, CD268, CD28, CD80, CD22, CD4, CD2, CD33, CD30, CD38, CD52, CD80, CD140b, PSMA, TYR, FCRL2, MUC17, GPR143, NMNAT2, MAGE, MAGEC2, MAGE-A3, MART-1, WT-1, EPHA2, KRT19, CLDN7, DKK1, FGF19, SCN3A, SCN2A, GAS1, S100Z, GAPT, GPR35, NY-ESO, cadherin 24, DLK1, GPR173, ALK, GFRA3, GUCY2C, DLL3, PSMA, PROX1, PSCA, glypican-1, mesothelin, (prostate stem cell antigen), GAGE-1 (G Antigen 1), ganglioside/GD2, GnT-V, β1,6-N (acetylglucosaminyltransferase-V), UPAR (urokinase-type plasminogen activator receptor), sialyl Lewis x (SLe^{x}) and sialyl Lewis a (SLe^{a})

26. The pharmaceutical composition for use according to claim 1, wherein the pharmaceutical composition is administered to a patient diagnosed with at least one type of cancer, wherein the treatment comprises subcutaneously administering at least one dose of said pharmaceutical composition.

27. The pharmaceutical composition for use according to claim 1, wherein said pharmaceutical composition is administered to said patient multiple times, preferably from about every 21 days, 28 days, to about 35 days, 36 days, 37 days, 38 days, 39 days, 40 days, 41 days, 42 days days, or every 22 days, 23 days, 24 days, 25 days, 26 days, 27 days.

28. The pharmaceutical composition for use according to claim 1, wherein the subcutaneous administration of said pharmaceutical composition increases the maximum tolerated dose (MTD) by at least 30%, 40%, 50% compared to the MTD of the pharmaceutical composition when administered intravenously.

29. Use of the conjugate of claim 1 in the manufacture of a pharmaceutical composition for subcutaneous administration.

30. A method of treating a patient afflicted with cancer, wherein the method comprises administering subcutaneously to said patient an effective dose of the pharmaceutical composition according to claim 1.

31. The method according to claim 30, wherein the pharmaceutical composition or the conjugate are administered at a single injection site.

32. The method according to claim 30, wherein the pharmaceutical composition or the conjugate are administered at a multiple injection sites.

33. The method according to claim 31, wherein the pharmaceutical composition is administered subcutaneously in a volume of about 0.5 ml, 0.75 ml, 1 ml to about 1.5 ml, 2 ml, 3 ml, 4 ml.

34. The method according to claim 30, wherein the effective dose of conjugate administered with the pharmaceutical composition is from about 100 μg/kg, 125 μg/kg, 150 μg/kg, 200 μg/kg body weight (b.w.) to about 250 μg/kg, 300 μg/kg, 350 μg/kg, 400 μg/kg, 450 μg/kg, 500 μg/kg, 600 μg/kg, 700 μg/kg, 750 μg/kg, 800 μg/kg, 900 μg/kg, 1 mg/kg, 1.25 mg/kg, 1.5 mg/kg, 1.75 mg/kg, 2 mg/kg body weight, or from about 175 μg/kg, 250 μg/kg, 275 μg/kg 300 μg/kg, 350 μg/kg, 475 μg/kg, 550 μg/kg, 75 μg/kg, 850 μg/kg, 1 mg/kg, 1.25 mg/kg, 1.5 mg/kg body weight to about 375 μg/kg, 425 μg/kg, 475 μg/kg, 950 μg/kg, 2.5 mg/kg, 3 mg/kg body weight.

35. The method according to claim 34, wherein the patient has failed prior standard-of-care first-line, second-line, or third-line cancer treatment for the respective cancer.
